(19) United States
(12) Patent Application Publication
(10) Pub. No.: US 2011/0251238 A1
(43) Pub. Date: Oct. 13, 2011

(54) PPAR AGONIST COMPOSITIONS AND METHODS OF USE

(75) Inventors: Jean-Louis Kraus, Marseille (FR); Olivier Blin, Marseille (FR); Frédéric Champavere, Paris (FR)

(73) Assignee: BIOPHARMED, Marseille (FR)

(21) Appl. No.: 13/140,514

(22) PCT Filed: Dec. 23, 2009
(86) PCT No.: PCT/IB09/55950

§ 371 Date: Jun. 17, 2011

Related U.S. Application Data
(60) Provisional application No. 61/242,497, filed on Sep. 15, 2009.

(30) Foreign Application Priority Data
Dec. 23, 2008 (FR) 08 07426

(51) Int. Cl. A61K 31/4709; C07D 401/12; C07D 409/14; C07D 405/14; A61P 3/04; A61P 29/00; A61P 27/02; A61P 25/00; A61P 25/16; A61P 37/06; A61P 25/18; A61P 9/10; A61P 9/00; A61P 3/06; A61P 25/22; A61P 15/08; A61P 13/12; G01N 33/566; A61K 31/47

(52) U.S. Cl. 514/314; 514/311; 546/179; 436/501

(57) ABSTRACT

Method for treating or preventing a PPAR-responsive condition in a subject, comprising administering to the subject a PPAR agonist that comprises a 8-hydroxyquinoline-methylene-N- group in an amount effective to activate a PPAR polypeptide.

### FIELD OF THE INVENTION

The present invention generally relates to the field of therapy. More particularly, the invention relates to compositions and methods for treating disease by stimulating Peroxisome proliferator-activated receptors (PPARs).

### BACKGROUND

PPARs regulate expression of target genes by binding to DNA response elements as heterodimers with the retinoid X receptor. These DNA response elements have been identified in the regulatory regions of a number of genes encoding proteins involved in glucose and lipid metabolism as well as energy balance. PPAR-γ agonists have shown promise in several therapeutic indications. Experience in the field of PPAR agonists has over the years shown various treatment modalities, e.g. methods of administration, formulations, doses, combination therapies) in which PPAR agonists can be used in various indications and settings.

Some but not all PPAR agonists have shown activity in cancer; it has been reported that in vitro anti-tumor effects appear to be structure specific and may be at least in part uncoupled from potency of PPAR activation. Anti-cancer activity of PPARγ agonists has been reported to include both PPARγ dependent and independent pathways. Generally, the growth inhibition on cancer cells by thiazolidinediones (TZDs; also called glitazones) analogues is linked to G1 phase cell cycle arrest and up-regulation of CDK inhibitors p21 and p27. Interestingly, TZDs with abolished PPARγ binding retained their anti-tumor activity. TZDs include troglitazone (Rezulin), rosiglitazone (Avandia), pioglitazone (Actos), and ciglitazone, which are all synthetic ligands for the PPARγ but which have differing and in some cases low anti-tumor activity. Additionally, the doses of TZD required to produce anti-tumor effects are three orders of magnitude higher than those required to modify insulin action (Day, (1999) Diabetic Med. 16:179-192). In parallel, numerous compounds have been reported that activate caspase-3/7, including 8-hydroxyquinolines in PCT publication no. WO2008/135671. Reports (e.g. WO2008/008767 for TZDs) have suggested that synergistic activity arises from the combination of PPARγ with caspase-3/7 activating agents such as chemotherapeutic agents taxol or etoposide. The need to combine pro-apoptotic compounds with PPAR-γ agonists presents complications, however, and it would be advantageous to identify PPAR-γ agonists that also have more potent inherent pro-apoptotic or cytostatic activity toward cancer cells.

In view of the importance of PPARs as biological targets for compounds used to help treat and prevent conditions such as metabolic disorders, diabetes, infection, neurodegenerative disorders, cancer, and others, there is a great need in the art for novel compounds capable of effectively and reliably activating PPARs in vitro and in vivo. The present invention addresses this and other needs.

### SUMMARY OF THE INVENTION

The present invention provides PPAR agonist compounds as well as their binding sites on PPARs. The present invention is based on studies where PPARγ was identified as a target for a first set of compounds initially selected on the basis of their anti-tumor and pro-apoptotic activity. The first set of compounds was used in docking studies to identify a binding site for agonists of PPAR and to develop related series of compounds as PPAR agonists. The fact that the compounds are active at PPAR designates them as suitable for use in the treatment of PPAR-responsive disorders, and for use according to treatment regimens adapted for PPAR agonists.

Additionally, the PPAR agonists have demonstrated advantageous properties in models of PPAR-responsive disorders compared to other PPAR agonists such as glitazones. For example, the PPAR agonists of the present invention are highly potent in their ability to activate caspase-3 and -7 in tumor cells and to induce apoptosis of tumor cells. The PPAR agonists are also more active in models of neuroprotection than the reference glitazone.

It is believed that the compounds' advantageous effects, e.g. in cancer and neuroprotection, is derived at least in part from the ability of the 8-hydroxyquinoline-methylene-N- scaffold to induce the formation of a quinone methide intermediate which alkylates nucleophilic biological entities such thiol, NH2, or OH, e.g. on proteins having thiol groups. The protonation of the tertiary amine (the N carrying the R1 and R2 groups), followed by addition of a nucleophile (either chemical or biological) on the H atom of the hydroxyquinoline in turn leads to the generation of a carbanionic entity, where resonance-induced stabilization of the entity then leads to a quinone-methide intermediate by breaking the C—N bond (in the aforementioned N atom), as shown in FIG. 2. The latter intermediate has potential alkylating activity, e.g. on biological substrates. In view of the greatly increased activity of bis-8-hydroxyquinoline compounds compared to mono-8-hydroxyquinoline compounds, it is believed that bis(8-hydroxyquinoline)methylene N- will be preferred for its alkylating power and ability to generate methide intermediate.

The PPAR agonists of the invention have an 8-hydroxyquinoline nucleus, unsubstituted or substituted, linked at the 4 position, though a methylene group, to an N-group. The N group may be linked to various moieties, e.g. a benzyl or a non-benzyl moiety such as a further 8-hydroxyquinoline group, unsubstituted or substituted, linked at the 4 position, though a methylene group, to the N group. Preferably, a PPAR agonist has a bis-8-hydroxyquinoline nucleus, unsubstituted or substituted, each 8-hydroxyquinoline group being linked at the 4 position, through a methylene group, to an N-group; these compounds are capable of alkylating protein and/or generating methide intermediate. PPAR agonists comprising a bis-8-hydroxyquinoline nucleus linked though a methylene group to an N-group are referred to as bis-8-hydroxyquinoline-methylene-N-compounds. In any embodiment, the PPAR agonist is or comprises a compound of Formulae I or III. In any embodiment, the PPAR agonist is or comprises 5,5′-(benzylazanediyl)bis(methylene)diquinolin-8-ol (2) (BPM18,725), 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5-(2-(Trifluoromethyl)benzylazanediyl)bis(methylene)diquinolin-8-ol (3) (BPM19,178), 5,5′-(3-(trifluoromethyl)benzylazanediyl)bis(methylene)diquinolin-8-ol (7) (BPM19,189), 5,5′-(3,5-bis(trifluorométhyl)benzylazanediyl)bis(méthylène)diquinoléin-8-ol (8) (BPM18,201), 5,5′-(thiophen-2-ylmethylazanediyl)bis(methylene)diquinolin-8-ol (11) (BPM18,202), 5,5′-(3-iodobenzylazanediyl)bis(methylene)diquinolin-8-ol (10) (BPM19,200), 5,5′-cyclohexylmethylazanediyl-bis-[(methylene)di(quinolin-8-ol)] (4) (BPM19,219) and 4-((bis((8-hydroxyquinolin-5-yl)methyl)amino)-methyl)cyclohexanecarboxylic acid (5) (BPM19,225). In any embodiment, a compound comprises a quinoline ring comprising a substitution (e.g. at the 2 and/or 7 position); optionally each quinoline ring in a PPAR agonist comprises a substitution; optionally, the substituent is a non-electron donating group, optionally further whereby the PPAR agonist retains the ability to generate a quinone-methide intermediate and display protein alkylating activity; optionally, the substituent is an electron donating group (e.g. a methyl group), optionally further whereby the PPAR agonist substantially lacks or has diminished ability to generate methide intermediate and thus protein alkylating activity but retains PPAR activating activity.

Preferably, PPAR agonists of the invention have a dual mechanism of action. One mechanism is by interacting with PPAR and activating PPAR signaling. A second mechanism comprises the alkylation of substrates, particularly thiol groups on proteins. Without wishing to be bound by theory, the alkylating mechanism may lead to accumulation of misfolded proteins in cancer and other cells and/or inducing a cellular stress response, and/or inducing oxidative stress in a cell and in turn inducing apoptosis in the cell. This mechanism may also underlie the ability of the compounds to activate caspase-3 and -7. Consequently, depending on the dose at which they are used, the PPAR agonists may render a cell more or less sensitive to a cellular insult or stress such as a cytotoxic agent, e.g. a pro-apoptotic agent, chemotherapeutic agent. Again, without wishing to be bound by theory, the PPAR agonists may have protective activity (e.g. in neurodegenerative disorders) by inducing an anti-inflammatory effect, by induction of protective stress response in cells, and/or by alkylating thiol radicals on proteins involved in exacerbating disease, e.g., thereby having greater neuroprotective potency than glitazones. This activity may be in addition to activity mediated by PPAR, as glitazone PPAR agonists have shown to inhibit macrophage and microglial activation that contributes to many neurodegenerative, ischaemic or inflammatory processes leading to neuronal death. In one aspect, the invention therefore provides a PPAR agonist compound comprising an 8-hydroxyquinoline-methylene-N- group, substituted or unsubstituted, wherein the compound is capable of modulating at least one PPAR-mediated cellular signaling pathway and is capable of alkylating a thiol group on a protein substrate.

The compounds will generally be used in the treatment of disease such that they exert at least PPAR agonism, with or without also having alkylating activity on thiol groups of proteins. Alkylating activity can be provided or avoided by selecting an appropriate treatment regimen (e.g. dosage) where the PPAR agonist exerts alkylating activity on proteins of interest, and/or by selecting a PPAR agonist compound that has higher or lower alkylating activity. The compounds may advantageously be used (e.g. in the treatment of cancer or in central nervous system (CNS) or neurodegenerative disorders where neuroprotection is beneficial) such that they have alkylating activity on thiol groups of proteins (e.g. that they generate a quinone-methide intermediate in the relevant context in vitro or in vivo). Optionally the compounds further have caspase-3 and/or -7 activation activity.

Accordingly, the present invention provides compounds comprising a 8-hydroxyquinoline nucleus, (e.g. a bis-8-hydroxyquinoline nucleus), unsubstituted or substituted, linked at the 4 position through a methylene group to an N-group compounds (e.g. compounds of Formulae I or III) having PPAR agonist activity, compositions which comprise them and methods for stimulating PPAR-mediated signaling. In some aspects, the compounds are capable of alkylating a thiol group on a protein and/or are capable of giving rise to a quinone-methide intermediate, in vitro or in vivo; in some aspects, the compounds are capable of stimulating PPARγ; in some embodiments, the compounds further have the ability to stimulate PPARδ; in some embodiments, the compounds further have the ability to stimulate PPARα; in some embodiments, the compounds further have caspase-3 and/or -7 activating activity; in some embodiments, the compounds further have the ability to stimulate RXRα. Included are compounds that have pan-activity across more than one PPAR polypeptide (e.g., PPARγ and PPARδ; PPARδ and PPARγ; PPARγ, PPARδ and PPARα), as well as compounds that have significant specificity (at least 5-, 10-, 20-, 50-, or 100-fold greater activity) on a single PPAR, or on two of the three PPARs (e.g. PPARγ over PPARδ, or PPARγ and PPARδ over PPARα).

In one aspect, the present invention provides methods for treating a PPAR-responsive condition in a subject, comprising administering to the subject an amount of a compound comprising a 8-hydroxyquinoline nucleus (preferably a bis-8-hydroxyquinoline nucleus), unsubstituted or substituted, linked at the 4 position through a methylene group to an N-group compounds, e.g. a compounds of Formulae I or III, effective to activate a PPAR (e.g. a PPARγ, PPARδ and/or PPARα), e.g. in PPAR-expressing cells. In one aspect, the compound is administered in an amount effective to alkylate thiol groups on a protein and/or in an amount effective to give rise to a quinone-methide intermediate, in vitro or in vivo. Optionally, the compound is administered in an amount effective to activate a RXRα polypeptide, e.g. in RXRα-expressing cells. Optionally the compound is administered in an amount effective to activate caspase-3 and/or -7. Optionally, the compound is administered in an amount effective to activate a PPAR and to alkylate proteins, and optionally further to activate caspase-3 and/or -7 and/or activate RXRα.

The compounds, compositions and methods described herein are useful for enhancing the activation of PPAR in PPAR-expressing cells (e.g. pancreatic islet cells; epithelial cells; endothelial cells; adipose tissue, the adrenal gland, spleen, and large colon and other tissues in which cells express high levels of PPARγ, cell lines HT22, HT-29, HCT116, MCF-7, U87, U373, neurons, astrocytes and oligodendrocytes, the latter expressing exclusively PPARδ, and others), in vitro and in vivo. Such compounds, compositions and methods are useful in a number of clinical applications, including as pharmaceutical agents and methods for treating or preventing PPAR-responsive conditions including non-cancer conditions (e.g. weight disorders, lipid disorders, metabolic disorders, cardiovascular disease, inflammatory or autoimmune diseases, neurodegenerative disorders, coagulation disorders, gastrointestinal disorders, genitourinary disorders, ophthalmic disorders, infections neuropathic or inflammatory pain, infertility, age-related macular degeneration) and cancers. The compounds of the invention can also be used in methods for assessing the effects of other compounds on PPAR activity, e.g., in assays to identify or characterize other candidate modulators of PPAR or of PPAR-expressing cells. The compounds and compositions are also useful in methods of inducing cellular differentiation, particularly by PPAR-expressing cells, arresting proliferation, sensitizing a cell to a pro-apoptotic or cytotoxic compound, and/or inducing apoptosis. Effects of compounds can be assessed for example in A549, BxPC3, LoVo, MCF7, PC3 or KB3 cells lines for adenocarcinomas, or in HS683, T98, GU373, U138, G19 or U87 cell lines in gliomas, or RhTP or B16F10 cell lines in melanoma.

In one embodiment, compounds of Formulae I or III, e.g. 5,5′-(benzylazanediyl)bis(methylene)diquinolin-8-ol (2) (BPM18,725), 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5-(2-(Trifluoromethyl)benzylazanediyl)bis(methylene)diquinolin-8-ol (3) (BPM19,178), 5,5′-(3-(trifluoromethyl)benzylazanediyl)bis(methylene)diquinolin-8-ol (7) (BPM19,189), 5,5′-(3,5-bis(trifluorométhyl)benzylazanediyl)bis(méthylène)diquinoléin-8-ol (8) (BPM18,201), 5,5′-(thiophen-2-ylmethylazanediyl)bis(methylene)diquinolin-8-ol (11) (BPM18,202), 5,5′-(3-iodobenzylazanediyl)bis(methylene)diquinolin-8-ol (10) (BPM19,200), 5,5′-cyclohexylmethylazanediyl-bis-[(methylene)di(quinolin-8-ol)] (4) (BPM19,219) and 4-((bis((8-hydroxyquinolin-5-yl)methyl)amino)-methyl)cyclohexanecarboxylic acid (5) (BPM19,225), are used in the treatment of pancreatic cancer. In one embodiment, compounds of Formulae I or III, e.g. 5,5′-(benzylazanediyl)bis(methylene)diquinolin-8-ol (2) (BPM18,725), 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5-(2-(Trifluoromethyl)benzylazanediyl)bis(methylene)diquinolin-8-ol (3) (BPM19,178), 5,5′-(3-(trifluoromethyl)benzylazanediyl)bis(methylene)diquinolin-8-ol (7) (BPM19,189), 5,5′-(3,5-bis(trifluorométhyl)benzylazanediyl)bis(méthylène)diquinoléin-8-ol (8) (BPM18,201), 5,5′-(thiophen-2-ylmethylazanediyl)bis(methylene)diquinolin-8-ol (11) (BPM18,202), 5,5′-(3-iodobenzylazanediyl)bis(methylene)diquinolin-8-ol (10) (BPM19,200), 5,5′-cyclohexylmethylazanediyl-bis-[(methylene)di(quinolin-8-ol)] (4) (BPM19,219) and 4-((bis((8-hydroxyquinolin-5-yl)methyl)amino)-methyl)cyclohexanecarboxylic acid (5) (BPM19,225), are used in the treatment of glioblastomas, melanomas or carcinomas.

The present invention further provides methods of using the PPAR agonist compounds in the treatment and prevention of disease. In view of the ability of the compounds to agonize PPAR, they can be used in protocols and treatment modalities (e.g. oral route in cancer and non-cancer diseases; parenteral, intravenous routes in, e.g. cancer; topical route in e.g. skin disorders, skin proliferative disorders, cancers, inflammation, etc.).

The PPAR agonists of the invention were effective when administered orally in an animal model; accordingly, in one aspect of the invention provides that the compounds may be administered orally, in an amount effective to activate a PPAR and/or in an amount effective to alkylate proteins and/or give rise to a quinone-methide intermediate. Optionally, the compound is administered in an amount effective to activate a PPAR (e.g. a PPARγ) and caspase-3 and/or -7 (e.g. in tumor cells), and optionally PPARδ, PPARα and/or RXRα. Optionally the compound is administered in an amount effective to caspase-3 and/or -7 activating activity and/or activate RXRα.

The PPAR agonists of the invention were effective when administered in an animal model of a CNS tumor and cross the blood brain barrier; accordingly one aspect of the invention provides that the compounds may be administered (e.g. outside the CNS, parenterally, orally, inhalation, transdermically), in an amount effective to activate a PPAR (e.g. a PPARγ) in the nervous system (e.g. CNS) of a subject and/or in an amount effective to alkylate proteins and/or give rise to a quinone-methide intermediate. Optionally, the PPAR agonist is administered in an amount effective to further activate caspase-3 and/or -7 and/or activate RXRα. In particular, compounds of the bis-8-hydroxyquinoline-methylene-N- class possessing PPAR-stimulating ability and a structure conferring alkylating activity were more potent than Temodar™ in an orthotopic model of glioblastoma where Hs683 cells were grafted orthotopically in mice, both by oral and parenteral routes.

In another embodiment, it was discovered that compounds of the invention are active in a model of infectious disease, consistent with PPAR agonism. Thus, in one embodiment compounds described herein can be used by in the treatment or prevention of infection, e.g. viral, bacterial, parasitic, or fungal infection, as well as any such infections that are resistant to treatment with one or more other therapeutic agents.

In another example, it was discovered that compounds of the invention are active in a model of neuroprotection, consistent with PPAR agonism. However, the bis-8-hydroxyquinoline-methylene-N- compounds in particular showed greater neuroprotective effect than the reference glitazone compound. Thus, in one embodiment compounds described herein can be used in the treatment or prevention of neurodegenerative disorders, e.g. Alzheimer's disease, Parkinson's disease, amyotrophic lateral sclerosis, spinal cord injury, and demyelinating disease. Moreover, it is demonstrated herein that the PPAR agonists are active in the brain following oral administration such that the PPAR agonists can be administered outside the CNS (e.g. parenterally, orally) to treat or prevent CNS disorders.

It was also found that these compounds' dual mechanism of action of PPAR activation and alkylating activity involves the presence of the tertiary amine (the N carrying the R1 and R2 groups in Formula I or carrying the Rc group in Formula III) and the H atom of the hydroxyquinoline; also bis-8-hydroxyquinoline-methylene-N-compounds had 10 fold greater alkylating activity than mono-5-methylene-8-hydroxyquinolines. Consequently, preferred compounds used to activate PPAR and alkylate substrates are bis-8-hydroxyquinoline-methylene-N-compounds, e.g. compounds for Formula I or III.

In a further aspect, the invention is based, at least in part, on the identification of an active site on a PPAR polypeptide such as PPARγ, which when bound by a 8-hydroxyquinoline compound, activates the PPAR polypeptide. In one aspect, the invention is directed to a method for identifying a candidate compound, e.g. a compound which modulates the activity of a PPAR polypeptide, a compound useful in therapy of a PPAR-responsive disease. The method comprises contacting a PPAR polypeptide with a 8-hydroxyquinoline compound, optionally a 8-hydroxyquinoline-methylene-N- compound (e.g. a compound which binds to an active site bound by (5,5′-(4-(trifluoromethyl)benzylazanediyl)bis(methylene)diquinolin-8-ol (6) (BPM18,708), - and/or 5,5′44-(methyl)benzylazanediyl)bis(methylene)diquinolin-8-ol (1) (BPM19,107) in the polypeptide) and detecting a modulated activity of the polypeptide, thereby identifying a candidate compound. Optionally, the method further comprises contacting assessing (e.g. detecting) whether the compound has alkylating activity or is capable of giving rise to a quinone-methide intermediate, thereby identifying a candidate compound. Optionally, the compound tested is a compound of Formulae I or III; optionally the compound is a bis-8-hydroxyquinoline-methylene-N-compound.

In a further aspect, the invention is directed to a method for identifying a candidate compound which modulates (e.g. activates) the activity of a PPAR polypeptide, such as PPARγ. For example, the method comprises: providing a three dimensional structure of an active site of the PPAR polypeptide bound by 5,5′-(4-(trifluoromethyl)benzylazanediyl)bis(methylene)diquinolin-8-ol (6) (BPM18,708) and/or 5,5′-(4-(methyl)benzylazanediyl)bis(methylene)diquinolin-8-ol (1) (BPM19,107), simulating a binding interaction between the active site and a candidate compound; and determining whether the candidate compound binds to one or more PPAR residues corresponding to Gly, Cys, or Arg 284; His, Leu, or Gln 266; Phe, Ala or Trp 204; Met, Ile or Val348; and/or Ile or Val 284 of the active site on respectively PPARγ, PPARα or PPARδ. Optionally the method comprises determining whether the candidate compound binds to one or more amino acid residues of the active site corresponding to residues S289, H323, H449 and Y473 on PPARγ, residues S280, Y314, H440 and Y464 on PPARα, residues H323 and H449 on PPARδ and/or residues R316 and/or A327 on RXRα. A compound is identified as a candidate compound when it is capable of binding to one or more of the amino acid residues of the active site. Optionally, the compound is a 8-hydroxyquinoline compound, optionally a 8-hydroxyquinoline-methylene-N- compound, optionally a compound of Formulae I or III.

In a further aspect, the invention is directed to a method for identifying a candidate compound which modulates (e.g. activates) the activity of a PPAR polypeptide, such as PPARγ, the method comprises: contacting a PPAR polypeptide with a 8-hydroxyquinoline compound, optionally a 8-hydroxyquinoline-methylene-N- compound, optionally a compound of Formulae I or III, and detecting binding of the compound to the polypeptide or detecting a modulated activity of the polypeptide. A compound is identified as a candidate compound when it is capable of binding to PPAR or modulating PPAR activity.

In a further aspect, the invention is directed to PPAR agonist compounds comprising a bis-8-hydroxyquinoline nucleus, unsubstituted or substituted, linked at the 4 position though a methylene group to an N-group compounds, e.g. a compounds of Formulae I or III, and compositions (e.g. pharmaceutical compositions) comprising them. In one embodiment, the invention is directed to PPAR agonist compounds of Formula III, and compositions (e.g. pharmaceutical compositions) comprising them. The invention further encompasses kits comprising any of the foregoing compounds and compositions.

### BRIEF DESCRIPTION OF THE DRAWINGS

FIG. 1 shows the cumulative proportion of mice surviving (y-axis) as a function of days post tumor graft (x-axis), in transgenic mice receiving orthotopic grafts of human glioblastoma cell lines and either 5,5′-(4-(trifluoromethyl)benzylazanediyl)bis(methylene)diquinolin-8-ol (6) (BPM18,708) or Temodar.

FIG. 2 shows a scheme whereby 5,5′-(4-(trifluoromethyl)benzylazanediyl)bis(methylene)diquinolin-8-ol (6) (BPM18,708) can give rise to a quinone-methide intermediate having alkylating activity.

### DETAILED DESCRIPTION OF THE INVENTION

The present invention is based, in part, on the discovery of a class of compounds that activate the biological activity of PPAR polypeptides, and that are capable of interacting in a glitazone-like binding pocket in the PPAR structure. Briefly, as described in the Examples, compounds comprising a bis-8-hydroxyquinoline nucleus, unsubstituted or substituted, linked at the 4 position though a methylene group to an N-group have PPARγ agonist activity in a functional assay. It was further discovered that discovered by modelling that the compounds are capable of being docked into a glitazone-like binding pocket in PPAR proteins, e.g. PPARγ, PPARδ and/or PPARα. Further, it was proposed that the compounds potency may arise from structural features including the tertiary amine (the N carrying the R1 and R2 groups) and the H atom of the hydroxyquinoline, which may lead to a quinine-methide intermediate having alkylating activity on chemical or biological substrates.

The binding site of 5,5′-(4-(trifluoromethyl)benzylazanediyl)bis(methylene)diquinolin-8-ol (6) (BPM18,708) and 5,5′-(4-(methyl)benzylazanediyl)bis(methylene)diquinolin-8-ol (1) (BPM19,107) was similar to the pioglitazone binding site. The binding pocket in PPARγ for the compounds tested was also discovered by modelling to correspond to residues 5289, H323, H449 and Y473 of the active site on PPARγ, residues 5280, Y314, H440 and Y464 of the active site on PPARα, residues H323 and H449 of the active site on PPARδ and residues R316 and/or A327 of the active site on RXRα. The residues defining these parts could be useful in designing novel chemical entities targeting the binding pocket in PPARγ described herein, or of a PPARγ-like protein.

The compounds were tested in various models of disease of PPAR-responsive disorders, including neurodegenerative disease, cancer and infectious disease. Consistent with their activity at PPAR, the compounds were effective in each PPAR-responsive disorder. The compounds were also effective orally. The compounds were as effective as PPAR agonist troglitazone, e.g. in neuroprotection, and as effective as the alkylating agent Temodar™ in glioblastoma, indicating that the compounds of the invention can be used in doses and administration regimens of glitazones, e.g. troglitazone, in PPAR-responsive disorders, and optionally in doses and administration regimens similar to that used for Temodar™ where alkylation of substrates is sought, in e.g., cancer, glioblastoma.

Several compounds—all having structures implying alkylating ability—displayed increased potency compared to reference glitazones. The increase in potency over glitazones varied in different types of cancer cells, as some compounds were more potent in some cell types. It is believed that the difference in activity (e.g. anti-tumor activity) may arise from different relative important of PPAR pathways in different cells, e.g. where some cells are more sensitive or express higher levels of one PPAR form over another. Compounds may have varying selectivity at different PPAR forms (e.g. PPARγ, PPARδ and/or PPARα), such that the optimal dose and compound will be determined as a function of the particular cellular target.

### DEFINITIONS

Unless otherwise defined, all technical and scientific terms used herein have the same meaning as commonly understood by one of ordinary skill in the art to which this invention belongs.

Throughout the specification, the word “comprise”, or variations such as “comprises” or “comprising” will be understood to imply the inclusion of a stated integer or groups of integers but not exclusion of any other integer or groups of integers.

The term “PPAR protein” refers to orphan nuclear receptors (ONR) from the orphan nuclear receptor family. Examples of this family of orphan nuclear receptors include but are not limited to PPARα, PPARγ, and PPARδ. The term “peroxisome proliferator activating receptor-γ” or “PPARγ” refers to the γ1, γ2 or γ3 isotypes or a combination of all isotypes of PPARγ.

The term “PPAR-like” refers to all or a portion of a molecule or molecular complex that has a commonality of shape and/or sequence identity to all or a portion of the PPAR protein. Typically, a PPARγ-like protein comprises a sequence segment which is at least 65% identical to the PPARγ of SEQ ID NO: 1, or a ligand-binding domain thereof. In specific and separate embodiments, the sequence identity between a sequence segment of a PPAR-like protein and the PPAR (or a ligand binding domain thereof) is at least 70%, at least 80%, at least 90%, at least 95%, or at least 99%. In one aspect of the invention, the PPAR-like protein is a PPAR homologue.

All residue numbers of the PPARγ, PPARα, PPARδ and RXRα structures described in the present patent specification use the numbering scheme as in SEQ ID NOS: 1, 2, 3 and 4, respectively. SEQ ID NO: 1 shows the 477 amino acid residue sequence PPARγ isoform 1 (differing from isoform 2 by the deletion of amino acids 1-27 of isoform 2), corresponding to SwissProt/UniProtKB accession no. P37231. SEQ ID NO:2 shows the 468 amino acid residue sequence of PPARα, corresponding to SwissProt/UniProtKB accession no. Q07869. SEQ ID NO: 3 shows the 477 amino acid residue sequence of isoform 1 of PPARδ corresponding to SwissProt/UniProtKB accession no. Q03181 and Genbank Accession nos. NP 619725 and NP_{—}619726. SEQ ID NO: 4 shows the 462 amino acid residue sequence RXRα, corresponding to SwissProt/UniProtKB accession no. P19793.

The term “homologue of PPAR” or “PPAR homologue” refers to a molecule that is homologous to a PPAR by structure or sequence. Examples of homologues include but are not limited to human PPARs and PPARs from other species with conservative substitutions, additions, deletions or a combination thereof or another member of the nuclear hormone receptor superfamily family, with conservative substitutions, additions, deletions or a combination thereof.

The term “binding pocket” refers to a region of a molecule or molecular complex that, as a result of its shape, electrostatic complementarity and hydrophobicity, favourably associates with another chemical entity or compound. The term “pocket” includes, but is not limited to, cleft, channel or site. PPAR, PPARγ or PPARγ-like molecules may have binding pockets which include, but are not limited to, peptide or substrate binding, lipid-binding, like the glitazone-binding pocket and antibody binding sites.

“BPM18,708-binding pocket” and “BPM19,107-binding pocket” respectively refers to a binding pocket of a molecule or molecular complex defined by the structure coordinates of a certain set of amino acid residues present in the PPARγ or PPARγ-like protein structure, as described herein.

The term “PPAR protein complex” or “PPAR homologue complex” refers to a molecular complex formed by associating a PPAR protein or PPAR homologue with a chemical entity. The term “molecular complex” or “complex” refers to a molecule associated with at least one chemical entity.

The term “associating with” refers to a condition of proximity between a chemical entity or compound, or portions thereof, and a binding pocket or binding site on a protein. The association may be non-covalent wherein the juxtaposition is energetically favoured by hydrogen bonding or by van der Waals or electrostatic interactions or it may be covalent.

The term “agonist” applies to a compound (ligand) that specifically binds and activates its target (cognate) receptor. For example, a PPARγ agonist specifically binds and activates a PPARγ isoform. Thus, a PPARγ agonist specifically binds PPARγ and activates downstream expression of a specific pattern of genes.

The term “PPAR responsive disorder” refers a disease or condition in which the biological function of a PPAR affects the development and/or course of the disease or condition, and/or in which modulation of PPAR alters the development, course, and/or symptoms of the disease or condition. Modulation (e.g. activation) of the level of activity of PPAR in a subject having a PPAR responsive disorder may reduce the severity and/or duration of the disease, reduces the likelihood, prevents or delays the onset of the disease or condition, and/or causes an improvement in one or more symptoms of the disease or condition.

The term “effective amount” indicates that the materials or amount of material is effective to achieve the desired effect, e.g. PPAR activation, activation of pro-apoptotic proteins, prevention, alleviation, or amelioration one or more symptoms of a disease or medical condition, and/or to prolong the survival of the subject being treated. The term “therapeutically effective” indicates that the materials or amount of material is effective to achieve a therapeutic effect.

The term “alkyl” refers to a linear or ramified alkyl, including but not limited to for example methyl, ethyl, propyl, butyl or isobutyl.

The term “alkenyl” refers to a linear or ramified alkenyl, in particular C₂-C₆ alkenyl, for example ethenyl or butenyl.

The term “alkynyl” refers to acetylenic derivatives, in particular C₂-C₆ acetylenic derivatives, for example ethynyl, propynyl or butynyl.

The term “cycloalkyl” refers to an alkyl ring such as cyclopropane, cyclobutane, cyclopentane or cyclohexane. A “heterocycloalkyl” refers to a cycloalkyl comprising one or several heteroatoms selected from N, O and S, such as for example pyrrolidine.

The term “aryl” refers a monocyclic or polycyclic aromatic carbon-based ring comprising between 5 and 14 carbon atoms, such as phenyl, naphthyl or cresyl. A “heteroaryl” refers to an aryl comprising one or several heteroatoms selected from N, O and S, such as pyridine, pyrimidine, pyrazine, furane, pyran, thipyran, thiophene.

### Compounds

Compounds according to the invention encompass compounds comprising a 8-hydroxyquinoline nucleus (e.g. a bis-, unsubstituted or substituted, linked at the 4 position, though a methylene group, to an N-group. Examples include the compounds of Formulae I and III. In any embodiment herein, a compound comprises a quinoline ring comprising a substitution (e.g. at the 2 and/or 7 position); optionally each quinoline ring in a PPAR agonist comprises a substitution (e.g. in one, two or three quinoline rings as may be present in compounds of Formulae I or III); in one embodiment, the substituent is other than an electron donating group, optionally further whereby the PPAR agonist retains the ability to generate methide intermediate and thus protein alkylating activity; optionally, the substituent is an electron donating group (e.g. a methyl group), optionally further whereby the PPAR agonist substantially lacks or has diminished ability to generate methide intermediate and thus protein alkylating activity but retains PPAR activating activity.

The compounds will generally have PPAR agonist activity; in some aspects, the compounds have the ability to stimulate PPARγ, PPARδ and/or PPARα; in some embodiments, the compounds further have caspase-3 and/or -7 activating activity; in some embodiments, the compounds further have the ability to stimulate RXRα; in some embodiments, the compounds further have the ability to stimulate PPARα. Compounds may have pan-activity across more than one PPAR polypeptide (e.g., PPARγ and PPARδ; PPARδ and PPARγ; PPARγ, PPARδ and PPARα), as well as compounds that have significant specificity (at least 5-, 10-, 20-, 50-, or 100-fold greater activity) on a single PPAR, or on two of the three PPARs (e.g. PPARγ over PPARδ, or PPARγ and PPARδ over PPARα). The compounds may furthermore bind an active site of the PPAR polypeptide bound by 5,5′-(4-(trifluoromethyl)benzylazanediyl)bis(methylene)diquinolin-8-ol (6) (BPM18,708) and/or 5,5′-(4-(methyl)benzylazanediyl)bis(methylene)diquinolin-8-ol (1) (BPM19,107), for example binding to one or more amino acid residues of the active site corresponding to residues S289, H323, H449 and Y473 on PPARγ, residues S280, Y314, H440 and Y464 on PPARα, residues H323 and H449 on PPARδ and/or residues R316 and/or A327 on RXRα. Optionally, the compounds are orally active. Optionally, the compounds are capable of crossing the blood-brain barrier.

PPAR agonists according to the invention includes compounds of formula (I),

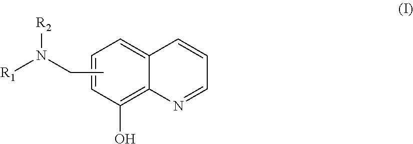

wherein the —CH₂—NR₁R₂ group is in the ortho, meta or para position relative to the —OH group, and in which:
one of the radicals R₁ and R₂ represents
a hydrogen atom, a C₁ to C₁₀ alkyl group, a C₂ to C₄ alkenyl or alkynyl group or a 5-methylene-8-hydroxyquinoline group; the other represents a 5-methylene-8-hydroxyquinoline group, a C₃ to C₆ cycloalkyl group, an aryl group, —(CH₂)ₙ-heteroaryl comprising one or more heteroatoms chosen from N, O and S, n being an integer between 0 and 4, a C₄ to C₆ group —(CH₂)ₙ-heterocycloalkyl in which the heteroatom represents N, O or S, n being an integer between 0 and 4, or alkylphenyl in which the alkyl represents C₁ to C₁₀, the cycloalkyl, aryl, heteroaryl, heterocycloalkyl and phenyl group being unsubstituted or substituted with 1 or 2 halogen atoms chosen from F, Br, I and Cl or with —CF₃, a C₁ to C₄ alkyl , COOH, CHO, COOR′ with R′ alkyl in C₁ to C₄;
or one of the radicals R₁ and R₂ represents a group of formula (II) linked to the asymmetric carbon

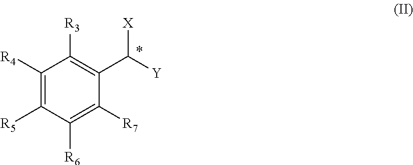

in which R₃, R₄, R₅, R₆ and R₇, independently of each other, represent a hydrogen atom, a C₁ to C₁₀ alkyl group, —CF₃, —NO₂, —NH₂, an N-5-methylene-8-hydroxyquinoline group, 1 or 2 halogen atoms chosen from F, Br, I and Cl or a group —O—R, R being a C₁ to C₄ alkyl group or —CF₃, X or Y represents a hydrogen atom, a C₁ to C₁₀ alkyl group, an aryl that is unsubstituted or substituted with a C₁ to C₁₀ alkyl group, —CF₃ or —NO₂,
the other of the radicals R₁ and R₂ representing an H atom, a tert-butoxycarbonyl group (Boc), 5-methylene-8-hydroxyquinoline or —(CH₂)ₙ-phenyl, n being an integer between 1 and 5;
or, when one of the groups R₁ and R₂ is a group Y—N—Y′ in which Y is chosen from the group formed by —(CH₂)ₙ—, n being an integer between 1 and 10 and —(CH₂)ₘ-phenyl-(CH₂)ₚ—, the phenyl being unsubstituted or substituted with 1 or 2 halogen atoms chosen from I, F, Br and Cl or with a C₁ to C₁₀ alkyl group, m and p being, respectively, integers between 1 and 4, and in which Y′ is 5-methylene-8-hydroxyquinoline, the other represents a hydrogen atom;
or, when one of the groups R₁ and R₂ represents a group —(CH₂)ₙ-naphthalene, n being an integer between 1 and 10, the naphthalene group being unsubstituted or substituted with one or more groups chosen from C₁ to C₁₀ alkyl groups, —CF₃ and —O—R in which R is a C₁ to C₁₀ alkyl group, the other is chosen from the group formed by a hydrogen atom, a 5-methylene-8-hydroxyquinoline group and a Boc group;
or R₁ and R₂ form a piperazine in which at least one of the carbon atoms of the ring is substituted with a C₁ to C₆ alkyl group and in which the N atom that is not part of the group —CH₂—NR₁R₂ is substituted with a 5-methylene-8-hydroxyquinoline group;
or R₁ and R₂ form a polyazamacrocycle (cyclam) representing unsubstituted 1,4,8,12-tetraazacyclopentadecane or 1,4,8,11-tetraazacyclotetradecane in which at least one of the N atoms of the ring in position 1, 4 and 8 is, independently, substituted with a Boc group, with a 5-methylene-8-hydroxyquinoline group or with —(CH₂)ₙ-phenyl-(CH₂)ₙ—Z, n being an integer between 1 and 10, in which Z represents one of the N atoms of a 1,4,8,12-tetraazacyclopentadecane or 1,4,8,11-tetraazacyclotetradecane in which the other N atoms of the ring in position 1, 4 and 8 are unsubstituted or are each independently substituted with a Boc group,
and pharmaceutically acceptable salts thereof, pharmaceutically acceptable solvates thereof, and enantiomers thereof.

Optionally, the compounds comprise a substitution in a quinoline ring; optionally the substitution is at the 2 and/or 7 position; optionally, the compound comprises two 8-hydroxyquinoline groups and a substitution in each 8-hydroxyquinoline group, optionally the compound comprises three 8-hydroxyquinoline groups and a substitution in two or three of the 8-hydroxyquinoline groups. Optionally the substituent is a group that is not electron donating.

According to one embodiment of the invention, one of the radicals R₁ and R₂ represents a hydrogen atom, a C₁ to C₆ alkyl group, a C₂ to C₄ alkenyl or alkynyl group or a 5-methylene-8-hydroxyquinoline group;

the other represents a 5-methylene-8-hydroxyquinoline group, an aryl group, —(CH₂)ₙ-heteroaryl comprising one or more heteroatoms chosen from N, O and S, n being an integer between 0 and 4, a C₄ to C₆ group —(CH₂)ₙ-heterocycloalkyl in which the heteroatom represents N, O or S, n being an integer between 0 and 4, or alkylphenyl in which the alkyl represents C₁ to C₆, the phenyl group being unsubstituted or substituted with 1 or 2 halogen atoms chosen from F, Br, I and Cl or with one or two —CF₃ groups;
or one of the radicals R₁ and R₂ represents a group of formula (II) linked to the asymmetric carbon

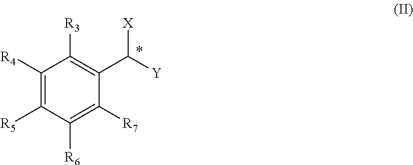

in which R₃, R₄, R₅, R₆ and R₇, independently of each other, represent a hydrogen atom, a C₁ to C₆ alkyl group, —CF₃, —NO₂, an N-5-methylene-8-hydroxyquinoline group, 1 or 2 halogen atoms chosen from F, Br, I and Cl or a group —O—R, R being a C₁ to C₃ alkyl group or —CF₃,
X or Y represents a hydrogen atom, a C₁ to C₆ alkyl group, an aryl that is unsubstituted or substituted with a C₁ to C₆ alkyl group, —CF₃ or —NO₂,
the other of the radicals R₁ and R₂ representing an H atom, a Boc group, 5-methylene-8-hydroxyquinoline or —(CH₂)ₙ-phenyl, n being an integer between 1 and 5;
or, when one of the groups R₁ and R₂ is a group Y—N—Y′ in which Y is chosen from the group formed by —(CH₂)ₙ—, n being an integer between 1 and 6, —(CH₂)ₘ-phenyl-(CH₂)ₚ—, the phenyl being unsubstituted or substituted with 1 or 2 halogen atoms chosen from F, Br and Cl or with a C₁ to C₆ alkyl group, m and p being, respectively, integers between 1 and 4, and in which Y′ is 5-methylene-8-hydroxyquinoline, the other represents a hydrogen atom;
or, when one of the groups R₁ and R₂ represents a group —(CH₂)ₙ-naphthalene, n being an integer between 1 and 6, the naphthalene group being unsubstituted or substituted with one or more groups chosen from C₁ to C₆ alkyl groups, —CF₃ and —O—R in which R is a C₁ to C₆ alkyl group, the other is chosen from the group formed by a hydrogen atom, a 5-methylene-8-hydroxyquinoline group and a Boc group;
or R₁ and R₂ form a piperazine in which at least one of the carbon atoms of the ring is substituted with a C₁ to C₄ alkyl group and in which the N atom that is not part of the group —CH₂—NR₁R₂ is substituted with a 5-methylene-8-hydroxyquinoline group;
or R₁ and R₂ form a polyazamacrocycle (cyclam) representing unsubstituted 1,4,8,12-tetraazacyclopentadecane or 1,4,8,11-tetraazacyclotetradecane in which at least one of the N atoms of the ring in position 1, 4 and 8 is, independently, substituted with a Boc group, with a 5-methylene-8-hydroxyquinoline group or with —(CH₂)ₙ-phenyl-(CH₂)ₙ—Z, n being an integer between 1 and 6, in which Z represents one of the N atoms of a 1,4,8,12-tetraazacyclopentadecane or 1,4,8,11-tetraazacyclotetradecane in which the other N atoms of the ring in position 1, 4 and 8 are unsubstituted or are each independently substituted with a Boc group,
and pharmaceutically acceptable salts thereof, pharmaceutically acceptable solvates thereof, and enantiomers thereof.

In another embodiment, one of the radicals R₁ and R₂ represents a hydrogen atom, a C₁ to C₄ alkyl group, a C₂ to C₄ alkenyl or alkynyl group or a 5-methylene-8-hydroxyquinoline group; the other represents a 5-methylene-8-hydroxyquinoline group, an aryl group, —(CH₂)ₙ-heteroaryl comprising one or more heteroatoms chosen from N, O and S, n being an integer between 0 and 3, a C₄ to C₆ group —(CH₂)ₙ-heterocycloalkyl in which the heteroatom represents N, O or S, n being an integer between 0 and 3, or alkylphenyl in which the alkyl represents C₁ to C₄, the phenyl group being unsubstituted or substituted with 1 or 2 halogen atoms chosen from F and I or with one or two —CF₃ groups;

or one of the radicals R₁ and R₂ represents a group of formula (II) linked to the asymmetric carbon

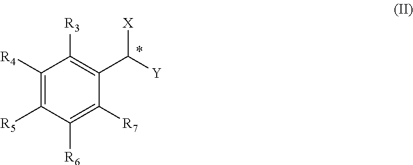

in which one of the radicals R₃, R₄, R₅, R₆ and R₇ represents an N-5-methylene-8-hydroxyquinoline group and the others represent a hydrogen atom,
X or Y represents a hydrogen atom, a C₁ to C₄ alkyl group, an aryl that is unsubstituted or substituted with a C₁ to C₄ alkyl group, —CF₃ or —NO₂,
the other of the radicals R₁ and R₂ representing H, a tert-butoxycarbonyl (Boc) group or 5-methylene-8-hydroxyquinoline;
or, when one of the groups R₁ and R₂ is a group Y—N—Y′ in which Y is chosen from the group formed by —(CH₂)ₙ—, n being an integer between 1 and 4, —(CH₂)ₘ-phenyl-(CH₂)ₚ—, the phenyl being unsubstituted or substituted with 1 or 2 halogen atoms chosen from F, Br and Cl or with a C₁ to C₄ alkyl group, m and p being, respectively, integers between 1 and 3, and in which Y′ is 5-methylene-8-hydroxyquinoline, the other represents a hydrogen atom;
or, when one of the groups R₁ and R₂ represents a group —(CH₂)ₙ-naphthalene, n being an integer between 1 and 4, the naphthalene group being unsubstituted or substituted with one or more groups chosen from C₁ to C₄ alkyl groups, —CF₃ and —O—R in which R is a C₁ to C₄ alkyl group, the other is chosen from the group consisting of a hydrogen atom, a 5-methylene-8-hydroxyquinoline group and a Boc group;
or R₁ and R₂ form a piperazine in which at least one of the carbon atoms of the ring is substituted with a C₁ to C₃ alkyl group and in which the N atom that is not part of the group —CH₂—NR₁R₂ is substituted with a 5-methylene-8-hydroxyquinoline group;
or R₁ and R₂ form a polyazamacrocycle (cyclam) representing unsubstituted 1,4,8,12-tetraazacyclopentadecane or 1,4,8,11-tetraazacyclotetradecane in which at least one of the N atoms of the ring in position 1, 4 and 8 is, independently, substituted with a Boc group, with a 5-methylene-8-hydroxyquinoline group or with —(CH₂)ₙ-phenyl-(CH₂)ₙ—Z, n being an integer between 1 and 4, in which Z represents one of the N atoms of a 1,4,8,12-tetraazacyclopentadecane or 1,4,8,11-tetraazacyclotetradecane in which the other N atoms of the ring in position 1, 4 and 8 are unsubstituted or are each independently substituted with a Boc group,
and pharmaceutically acceptable salts thereof, pharmaceutically acceptable solvates thereof, and enantiomers thereof.

Examples of PPAR agonists according to the invention also include compounds of Formula (III),

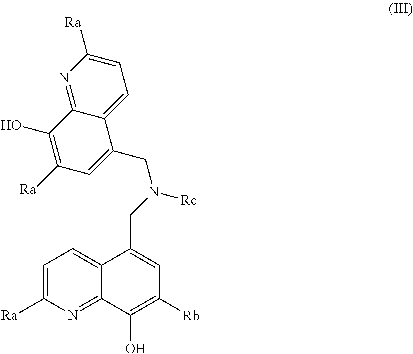

in which:
each Ra and each Rb independently of each other represent a C₁-C₆ alkyl group, a C₃-C₆ cycloalkyl group, a phenyl group, an allyl group, a C₂ to C₄ alkenyl or alkynyl group, a propargyl or benzyl group, preferably a [propene-1-yl] group, each of the alkyl, cycloalkyl, phenyl, allyl, propargyl or benzyl groups being unsubstituted or substituted (e.g. with a halogen atom), or I, Br , Cl , F or NH2, NO2, or O—R, in which R could be a C₁ to C₆ (or optionally C₁ to C₄) alkyl group, a C₃-C₆ cycloalkyl group, a substituted or unsubstituted phenyl group, or a ω-substituted (carboxylic or amino groups) alkyl chain;
one of Ra and Rb can be hydrogen (so that substitution on the 8-hydroxyquinoline ring can be on positions 2 and/or 7 of the ring).
Rc represents a hydrogen atom, a C₁ to C₁₀ alkyl group in, a C₂ to C₄ alkenyl or alkynyl group or a 5-methylene-8-hydroxyquinoline group, a C₃ to C₆ cycloalkyl group, an aryl group, a —(CH₂)ₙ-heteroaryl comprising one or several heteroatoms selected from N, O and S, n being an integer between 0 and 4, a C₄ to C₆ —(CH₂)ₙ-heterocycloalkyl group in which the heteroatom represents N, O and S, n being an integer between 0 and 4, or alkylphenyl where the alkyl represents C₁ to C₁₀, the cycloalkyl, aryl, heteroaryl, heterocycloalkyl and phenyl groups being unsubstituted or substituted with one or two groups selected from F, Br, I and Cl, —CF₃, a C₁ to C₄ alkyl , COOH, CHO, COOR′ where R′ is a C₁ to C₄ alkyl group;
or Rc represents a group of formula (II) linked to the asymmetric carbon

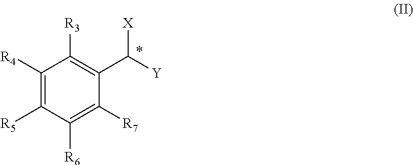

in which R₃, R₄, R₅, R₆ and R₇, independently of each other, represent a hydrogen atom, a C₁ to C₁₀ alkyl group, —CF₃, —NO₂, —NH₂, an N-5-methylene-8-hydroxyquinoline group, 1 or 2 halogen atoms chosen from F, Br, I and Cl or a group —O—R, R being a C₁ to C₄ alkyl group or —CF₃, X or Y represents a hydrogen atom, a C₁ to C₁₀ alkyl group, an aryl that is unsubstituted or substituted with a C₁ to C₁₀ alkyl group, —CF₃ or —NO₂,
or Rc represents a tert-butoxycarbonyl (Boc) group or —(CH₂)ₙ-phenyl, n being an integer between 1 and 5;
or Rc represents a Y—N—Y′ group where Y is selected from the group consisting of —(CH₂)ₙ—, n being an integer between 1 and 10, —(CH₂)ₙ-phenyl-(CH₂)ₚ—, the phenyl being unsubstituted or substituted with 1 or 2 halogen atoms selected from F, Br, I and Cl or with a C₁ to C₁₀ alkyl group, m and p respectively being number between 1 and 4, and wherein Y′ is 5-methylene-8-hydroxyquinoline;
or Rc represents a —(CH₂)ₙ-naphtalene group, n being an integer between 1 and 10, the naphthalene group being unsubstituted or substituted with one or several groups selected from C₁ to C₁₀ alkyl groups, —CF₃ and O—R where R is a C₁ to C₁₀ alkyl group
and pharmaceutically acceptable salts thereof, pharmaceutically acceptable solvates thereof, and enantiomers thereof.

Optionally, in any of the embodiments, the Y—N—Y′N may be substituted with a hydrogen atom; C₁ to C₁₀ alkyl group, C₂ to C₅ cycloalkyl group, an aryl group (e.g. a phenyl, benzyl, substituted phenyl (e.g. substituted with Cl, Br NO2, NH2, I, Omethyl), heterocyclic moieties (pyridinyl, thiophenyl, oxazolyl) or a hydroxyl group.

Optionally, in any of the embodiments herein, each Ra represents a C₁-C₆ alkyl group, unsubstituted or substituted with a halogen atom, or Ra represents a —NO₂, NH₂ or —OR group where R is a C₁ to C₄ alkyl group.

Optionally, in any of the embodiments herein, each Rb represents an allyl group, a C₂ to C₄ alkenyl or alkynyl group, propargyl or benzyl, preferably a [propene-1-yl] group, the allyl, propargyl or benzyl being unsubstituted or substituted with a halogen atom, a —NO₂, NH₂ or —OR group where R is a C₁ to C₄ alkyl group.

In one embodiment, Rb represents an allyl group, a propargyl or benzyl substituted with a F, I, Cl or Br.

In one embodiment, Rc represents a —(CH₂-(2-[thiophen], —(CH₂-([tetrahydrofuran]), —(CH₂-4-(cyclohexanecarboxylic acid), —(CH₂-(1-methyl-1H-[pyrrole]), 2-([pyrrolidin]-1-yl)ethyl, or 2-[pyridine-2-yl)ethyl] group.

In one embodiment, Rc represents a —CH₂-phenyl group, the phenyl group being unsubstituted or substituted at ortho, meta or para positions with one or several —CF₃, —CH₃, —NH₂, —OCH₃, F, Br, Cl, I.

In one embodiment, Rc represents a —CH₂-phenyl group, the phenyl group being substituted at meta position with —CF₃.

In any of the embodiments herein, R1 and/or R2 in Formula I or Rc in Formula III can optionally be selected to be a group other than a propargyl group. Furthermore, in any of the embodiments herein, a Formula or PPAR agonist may optionally specifically exclude any of the compounds selected from the group consisting of: 5-((benzylamino)methyl)quinolin-8-ol; 5-((1,4,8,12-tetraazacyclopentadecan-8-yl)methyl)quinolin-8-ol; tri-tert-butyl 12-((8-hydroxyquinolin-5-yl)methyl)-1,4,8,12-tetraazacyclopentadecane-1,4,8-tricarboxylate; tri-tert-butyl 11-((8-hydroxyquinolin-5-yl)methyl)-1,4,8,11-tetraazacyclotetradecane-1,4,8-tricarboxylate; 5-((1,4,8,11-tetraazacyclotetradecan-1-yl)methyl)quinolin-8-ol; tri-tert-butyl 11-(3-((4,11-bis(tert-butoxycarbonyl)-8-((8-hydroxyquinolin-5-yl)methyl)-1,4,8,11-tetraazacyclotetradecan-1-yl)methyl)benzyl)-1,4,8,11-tetraazacyclotetradecane-1,4,8-tricarboxylate; 5,5′-(propane-1,3-diylbis(azanediyl))bis(methylene)- diquinolin-8-ol; 5-((8-(4- ((1,4,8,11-tetraazacyclotetradecan-1-yl)methyl)benzyl)-1,4,8,11-tetraazacyclotetradecan-1-ylmethyl)quinolin-8-ol; di-tert-butyl 4,8-bis((8-hydroxyquinolin-5-yl)methyl)-1,4,8,11-tetraazacyclotetradecane-1,11-dicarboxylate; 5,5′-(1,4,8,11-tetraazacyclotetradecane-1,11-diyl)bis(methylene)diquinolin-8-ol; 5,5′-(1,4-phenylenebis(methylene))bis(azanediyl)-bis(methylene)diquinolin-8-ol; 5-(((8-hydroxyquinolin-5-yl)(4-methylbenzyl)amino)methyl)quinolin-8-ol; tert-butyl 8-hydroxyquinolin-5-yl(4-methylbenzyl)-carbamate; 5-((4-methylbenzylamino)methyl)quinolin-8-ol; 5-(naphthalen-1-ylmethylamino)quinolin-8-ol; 5,5′-(naphthalen-1-ylmethylazanediyl)diquinolin-8-ol; tert-butyl 8-hydroxyquinolin-5-yl(naphthalen-1-ylmethyl)carbamate; and 5(((8-hydroxyquinolin-5-yl)(4-(trifluoromethyl)benzyl)amino)methyl)quinolin-8-ol.

Further examples of PPAR agonists according to the invention also include compounds of Formula (III), above, in which Ra and Rb each represent a hydrogen atom, and each Rc represents a substituent selected from the group consisting of: C₁ to C₁₂ alkyl; C₄ to C₈ cycloalkyl; cyclohexyl methyl (BPM19,219); 4-Carboxycyclohexylmethyl (BPM19,225); 6-hydroxyhexyl (BPM19,232); 2,3-dihydro-1H inden-1-yl (BPM19,899); 2-(pyrrolidin-1-yl)ethyl (BPM19,214); tetrahydrofuran-2-ylmethyl (BPM19,197); 1-methyl-1-H-pyrrol-2-yl)methyl (BPM19,216); an allyl group (BPM19,900); a propargyl group (BPM19,905); a group comprising an aryl substituent; 4-trifluoromethyl-phenoxy-2 (BPM19,897); 4-ethoxy-1-(4-hydroxyphenyl)-4-oxobutan-2-yl (BPM19,902); 1H-benzo[d]imidazol-2-yl)-methyl (BPM19,228); pyridin-4-yl-methyl(BPM19,226); benzihydryl (BPM19,886) iodobenzyl (BPM19,200); 4-trifluoromethoxybenzyl (BPM19,205); 2-trifluoromethyl benzyl (BPM19,178); 4-trifluoromethylbenzyl, 3-trifluoromethylbenzyl; 3,5-ditrifluoromethylbenzyl (BPM18,201); 2-methylbenzyl; 3-methylbenzyl; 4-methylbenzyl (BPM19,107); benzyl (BPM18,725); naphthalen-1-ylmethyl (BPM19,702); 4-nitro benzyl (BPM19,177); 3-nitrobenzyl; 2-nitrobenzyl; 4-aminobenzyl (BPM19,870); thiophene-2-ylmethyl (BPM18,202); 2(pyridin-2-yl)ethyl (BPM19,193); (R)1-phenylethyl (BPM19,129); (S)-1-phenylethyl; and 8-hydroxyquinolin-5-yl)-methyl (BPM 19,211).

Further examples of PPAR agonists according to the invention also include compounds of Formula (III), above, in which Ra, Rb and/or Rc represent a substituent other than a hydrogen atom. In one embodiment, Rc represents a substituent selected from the group consisting of a 4-methylbenzyl, a 4-trifluoromethylbenzyl and a 4-trifluoromethyl. In one embodiment, Ra and/or Rb represent a substituent selected from the group consisting of a hydrogen atom, a halogen atom (e.g. an I) and a methyl group. In one embodiment, the PPAR agonist is a compound of Formula (III), above, where:

Rc=4-methylbenzyl, Ra=H, Rb=I (BPM19,888);
Rc=4-trifluoromethylbenzyl, Ra=H, Rb=I (BPM19,887);
Rc=4-trifluoromethyl, Ra=methyl, Rb=H (BPM19,230);
Rc=4-trifluoromethylbenzyl, Ra=H, Rb=methyl (BPM19,876);
Rc=4-methylbenzyl, Ra=H, Rb=methyl;
Rc=4-trifluoromethyl benzyl, Ra=I, Rb=H or Rc=4-methylbenzyl, Ra=I, Rb=H

Further examples of PPAR agonists according to the invention include the following compounds.
5-((1,4,8,12-tetraazacyclopentadecan-8-yl)methyl)quinolin-8-[6]ol, (BPM18,994)
tri-tert-butyl-12((8-hydroxyquinolin-5-yl)methyl)-1,4,8,12-tetraazacyclopentadecane-1,4,8-tricarboxylate, (BPM 19,008)
tri-tert-butyl-11((8-hydroxyquinolin-5-yl)methyl)-1,4,8,11-tetraazacyclotetradecane-1,4,8-tricarboxylate, (BPM19,048)
5-((1,4,8,11-tetraazacyclotetradecane-1-yl)methyl)quinolin-8-ol, (BPM19,009)
tri-tert-butyl-11-(3-((4,11-bis(tert-butoxycarbonyl)-8-((8-hydroxyquinolin-5-yl)methyl)-1,4,8,11-tetraazacyclotetradecane-1-yl)methyl)benzyl)-1,4,8,11-tetraazacyclotetradecane-1,4,8-tricarboxylate, (BPM19,076)
5,5′-(propane-1,3-diylbis(azanediyl))bis(methylene)-diquinolin-8-ol, (BPM19,077)
5-((8-(4-((1,4,8,11-tetraazacyclotetradecan-1-yl)methyl)benzyl)-1,4,8,11-tetraazacyclotetradecan-1-yl)methyl)quinolin-8-ol, (BPM19,078)
5,5′-(piperazine-1,4-diylbis(methylene))diquinolin-8-ol, (BPM19,090)
di-tert-butyl-4,8-bis((8-hydroxyquinolin-5-yl)methyl)-1,4,8,11-tetraazacyclotetradecane-1,11-dicarboxylate, (BPM19,089)
5,5′-(1,4,8,11-tetraazacyclotetradecane-1,11-diyl)bis(methylene)diquinolin-8-ol, (BPM19,094)
5,5′-(1,4-phenylenebis(methylene))bis(azanediyl)-bis(methylene)diquinolin-8-ol, (BPM 19,097)
5-((benzylamino)methyl)quinolin-8-ol, (BPM18,726)
54-(((8-hydroxyquinolin-5-yl)(4-methylbenzyl)amino)-methyl)quinolin-8-ol,
tert-butyl-8-hydroxyquinolin-5-yl(4-methylbenzyl)-carbamate, (BPM19,113)
5-((4-methylbenzylamino)methyl)quinolin-8-ol, (BPM19,114)
5-(naphthalen-1-ylmethylamino)quinolin-8-ol, (BPM18,722)
5,5′-(naphthalen-1-ylmethylazanediyl)diquinolin-8-ol, (BPM19,702)
tert-butyl-8-hydroxyquinolin-5-yl(naphthalen-1-ylmethyl)carbamate, and
5-(((8-hydroxyquinolin-5-yl)(4-(trifluoromethyl)benzyl)amino)methyl)quinolin-8-ol,
5,5′-(benzylazanediyl)bis(methylene)diquinolin-8-ol (BPM18,725) (2),
5,5′-(4-(methyl benzylazanediyl)bis(methylene)diquinolin-8-ol (BPM19,107) (1),
5,5′-(4-(trifluoromethyl benzylazanediyl)bis(methylene)diquinolin-8-ol (BPM18,708),
5,5′-(2-(trifluoromethyl benzylazanediyl)bis(methylene)diquinolin-8-ol (BPM19,078),
5,5′-(3-(trifluoromethyl benzylazanediyl)bis(methylene)diquinolin-8-ol (BPM19,189),
5,5′-(3,5-bis(trifluoromethyl benzylazanediyl)bis(methylene)diquinolin-8-ol (BPM18,201),
5,5′-(3-[iodo]benzylazanediyl)bis(methylene)diquinolin-8-ol (BPM19,200),
5,5′-([thiophen]-2-ylmethylazanediyl)bis(methylene)diquinolin-8-ol (BPM18,202), and
4-((bis((8-hydroxyquinolin-5-yl)methyl)amino)-methyl)cyclohexanecarboxylic acid (BPM19,225).

In one embodiment, a PPAR agonist compound may specifically include or exclude a compound or chemical formula disclosed in PCT Publication no. WO2008/135671, the disclosure of which is incorporated herein by reference in its entirety. Compounds may be e.g. compounds according to Formula I in which a substituent is specified, such as a group not described in WO2008/135671, by the presence of two substituents such as for example a substitution with 3,5di(trifluoromethyl), or compounds of Formula III, as described in French patent application no. 0807426 filed on 23 Dec. 2008, the disclosure of which is incorporated herein by reference in its entirety. Compounds of Formula III generally differ from compounds of Formula I by substitution on the 8-hydroxyquinoline ring (substitutions on positions 2 and/or 7 of the ring).

Compounds of Formula I or III can be prepared according to standard methods (e.g. according to methods described in WO2008/135671) or according to the following protocol. Briefly, the amine corresponding to the compound desired (2.87 nmol) is added to a stirred solution of dihydrochloride of 5-chloromethylquinoline-8-ol (5.74 nmol) in CH₃CN (20 ml). The mixture is heated at 50° C. overnight and the reaction assessed by thin layer chromatography (TLC). The mixture is cool to 0° C. and filtered; the filtrate is washed with 10 mL cold CH₃CN. The residue is purified by chromatography on silica gel (CH₂Cl₂/MeOH 95.5 as eluent). The preparation of a compound of Formula III is similar, the starting solution being a solution of substituted 5-chloromethylquinoline-8-ol, either 2-substituted or 7 substituted, or 2,7 substituted. Salts can be prepared according to standard methods; for example salts can be obtained by reacting a mineral base such as lithium sodium or potassium hydroxide, or sodium or potassium carbonate, with a compound of Formulae I or III in acid form. Salts of mineral acids such as phosphorus derivatives can be used similarly, as can salts of organic acids such as sodium acetate and any organic amine base such as triethylamine or diethylamine. The compounds may be used as a pharmaceutically acceptable solvate, e.g. a pharmaceutically acceptable hydrate of a compound of Formulae I or III.

In any embodiment herein, the PPAR agonist may be selective for PPARγ and optionally PPARδ and/or PPARα. In some embodiments, compounds are preferably selective for PPARγ. Such selectivity means that the compound has at least 5-fold greater activity (preferably at least 10-, 20-, 50-, or 100-fold or more greater activity) on the specific PPAR(s) than on the other PPAR(s), where the activity is determined using a biochemical assay suitable for determining PPAR activity, e.g., any assay known to one skilled in the art or as described herein. In some embodiments, compounds have significant activity on PPARδ and PPARγ.

As demonstrated herein, the compounds of the invention, e.g. Formulae I or III, have potent PPAR agonist activity as well as potent anti-tumor activity, e.g. in pancreatic cancer, gliomas, including the inhibition of cancer cell proliferation and migration. The compounds thus inhibit the proliferation, viability and survival or cancer cells. The presence of two 5-methylene-8-hydroxyquinoline substituents in the compounds of the invention conferred an extremely potent biological activity, particularly anti-tumor and pro-apoptotic activity. In particular, the bis-5-methylene-8-hydroxyquinolines (e.g. compounds of Formula I where one of R1 and R2 represent a 5-methylene-8-hydroxyquinoline group, and compounds of Formula III) have markedly higher anti-cancer activity that equivalent mono-5-methylene-8-hydroxyquinoline compounds. IC₅₀ values in anti-tumor assays (the concentration of the compound at which 50% of tumor cells tested survived treatment) were far lower, up to a more than 10-fold difference, for the bis-5-methylene-8-hydroxyquinolines compared to mono-5-methylene-8-hydroxyquinolines.

In some embodiments, a PPAR agonist, e.g. a compound of Formulae I or III, will have an EC₅₀ of less than 100 nM, less than 50 nM, less than 20 nM, less than 10 nM, less than 5 nM, or less than 1 nM with respect to at least one of PPARγ and/or PPARδ and/or PPARα as determined in a generally accepted PPAR activity assay. In some embodiments, a compound of the invention may be a selective agonist of PPARγ over other PPAR polypeptides.

In some embodiments of the invention, the compounds of the invention also have desirable pharmacologic properties. In some embodiments the desired pharmacologic property is PPAR pan-activity, PPAR selectivity for any individual PPAR (PPARδ and/or PPARγ), activation of pro-apoptotic proteins (e.g. activation of caspase 3 activity), or any one or more of serum half-life longer than 2 hr, also longer than 4 hr, also longer than 8 hr, aqueous solubility, and oral bioavailability more than 10%, also more than 20%.

### BPM18,708- and BPM19,107-Binding Pockets of PPARs

As disclosed above, applicants have used a three-dimensional model of binding of PPAR-BPM18,708 and BPM19,107 complexes. The chemical structures of the invention may in one aspect be useful for inhibitor or activator design for novel drugs to be used in the treatment of PPAR-responsive diseases or conditions, and to study the role of PPAR in cell signalling.

Binding pockets are of significant utility in fields such as drug discovery. The association of natural compounds BPM18,708 and BPM19,107 with the binding pockets in PPAR is believed to be the basis of their biological mechanisms of action. An understanding of such associations will help lead to the design of drugs having more favorable associations with their target receptor, and thus, improved biological effects. Therefore, this information is valuable in designing potential modulators (e.g. agents that activate) PPAR polypeptides.

In one aspect, the BPM18,708- and BPM19,107-binding pocket in PPARγ is defined by three-dimensional structure coordinates of a set of amino acids that comprises amino acid residues corresponding to or comprising one, two, three or four of the residues S289, H323, H449 and Y473, as numbered in SEQ ID NO: 1. In one aspect, the BPM18,708 and BPM19,107-binding pocket in PPARα is defined by three-dimensional structure coordinates of a set of amino acids that comprises amino acid residues corresponding to or comprising one, two, three or four of the residues S280, Y314, H440 and Y464, as numbered in SEQ ID NO: 2. In one aspect, the BPM18,708- and BPM19,107-binding pocket in PPARδ is defined by three-dimensional structure coordinates of a set of amino acids that comprises amino acid residues corresponding to or comprising one, two or three of the residues H323 and H449, as numbered in SEQ ID NO: 3. In one aspect, the BPM18,708- and BPM19,107-binding pocket in RXRα is defined by three-dimensional structure coordinates of a set of amino acids that comprises amino acid residues corresponding to R316 and/or A327 as numbered in SEQ ID NO: 4.

In one embodiment, the invention provides a compound of Formulae I or III, wherein said compound binds a binding pocket in PPARγ, PPARδ, PPARα and/or RXRα defined by three-dimensional structure coordinates of a set of amino acids as described herein.

In one embodiment of any of the preceding aspects, the PPARγ or PPAR-like protein molecule comprises an amino acid sequence at least 65% identical to a sequence of at least 50, 60 or 100 residues of, or all of, any one of SEQ ID NOS 1, 2 or 3.

### Design of Compounds

The design of compounds that bind a BPM18,708- and/or BPM19,107-binding pocket in PPAR (i.e. PPARγ, PPARδ or PPARα) or a PPAR-like polypeptide according to this invention generally involves consideration of two factors. First, the chemical entity must be capable of physically and structurally associating with parts or the entire BPM18,708- and/or BPM19,107-binding pocket. Non-covalent molecular interactions important in this association include hydrogen bonding, van der Waals' interactions, hydrophobic interactions and electrostatic interactions.

Second, the chemical entity must be able to assume a conformation that allows it to associate with the PPAR, PPARγ or PPARy-like BPM18,708- and/or BPM19,107-binding pocket directly. Although certain portions of the chemical entity will not directly participate in these associations, those portions of the chemical entity may still influence the overall conformation of the molecule. This, in turn, may have a significant impact on potency. Such conformational requirements include the overall three-dimensional structure and orientation of the chemical entity in relation to all or a portion of the BPM18,708- and/or BPM19,107-binding pocket, or the spacing between functional groups of a chemical entity comprising several chemical entities that directly interact with the PPAR or PPAR-like BPM18,708- and/or BPM19,107-binding pockets.

The potential inhibitory or binding effect of a chemical entity on a BPM18,708- and/or BPM19,107-binding pocket may be analyzed prior to its actual synthesis and testing by the use of computer modelling techniques. If the theoretical structure of the given entity suggests insufficient interaction and association between it and the BPM18,708- and/or BPM19,107-binding pocket, testing of the entity is obviated.

However, if computer modelling indicates a strong interaction, the molecule may then be synthesized and tested for its ability to bind to a BPM18,708- and/or BPM19,107-binding pocket. This may be achieved by testing the ability of the molecule to bind and/or inhibit/activate a PPAR protein such as PPARγ or a PPAR-like protein using the assays described above. In this manner, synthesis of inoperative compounds may be avoided.

A potential modulator of a BPM18,708- and/or BPM19,107-binding pocket of, e.g., PPARγ, may be computationally evaluated by means of a series of steps in which chemical entities or fragments are screened and selected for their ability to associate with the PPAR BPM18,708- and/or BPM19,107-binding pocket.

One skilled in the art may use one of several methods to screen chemical entities or fragments for their ability to associate with a BPM18,708- and/or BPM19,107-binding pocket of, e.g., PPARγ.

This process may begin by visual inspection of, for example, a PPARγ BPM18,708- and/or BPM19,107-binding pocket on the computer screen based on the PPARγ structure coordinates or other coordinates which define a similar shape generated from the machine-readable storage medium. Selected fragments or chemical entities may then be positioned in a variety of orientations, or docked, within that BPM18,708- and/or BPM19,107-binding pocket. Docking may be accomplished using software such as QUANTA (Accelrys Inc., San Diego, ®2001, 2002) and Sybyl (Tripos Associates, St. Louis, Mo.), followed by energy minimization and molecular dynamics with standard molecular mechanics force fields, such as CHARMM and AMBER.

Specialized computer programs may also assist in the process of selecting fragments or chemical entities. These include:
1. GRID (P. J. Goodford, “A Computational Procedure for Determining Energetically Favorable Binding Sites on Biologically Important Macromolecules”, Med. Chem., 28, pp. 849-857 (1985)). GRID is available from Oxford University, Oxford, UK.
2. MCSS (A. Miranker et al., “Functionality Maps of Binding Sites: A Multiple Copy Simultaneous Search: Structure, Function and Genetics, 11, pp. 29-34 (1991)). MCSS is available from Molecular Simulations, San Diego, Calif.
3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.
4. DOCK (I. D. Kuntz et al., “A Geometric Approach to Macromolecule-Ligand Interactions”, J. MoI. 161, pp. 269-288 (1982)). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of PPARγ. This would be followed by manual model building using software such as QUANTA (Accelrys Inc., San Diego, ®2001, 2002) or Sybyl (Tripos Associates, St. Louis, Mo.).

Useful programs to aid one of skill in the art in connecting the individual chemical entities or fragments include:
1. CAVEAT (P. A. Bartlett et al., “CAVEAT : A Program to Facilitate the Structure- Derived Design of Biologically Active Molecules”, in Molecular Recognition in Chemical and Biological Problems, Special Pub., Royal Chem. Soc, 78, pp. 182-196 (1989); G. Lauri and P. A. Bartlett, “CAVEAT: a Program to Facilitate the Design of Organic Molecules”, Comput. Aided MoI. Des., 8, pp. 51-66 (1994)). CAVEAT is available from the University of California, Berkeley, Calif.
2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).
3. HOOK (M. B. Eisen et al., “HOOK : A Program for Finding Novel Molecular Architectures that Satisfy the Chemical and Steric Requirements of a Macromolecule Binding Site”, Proteins: Struct., Funct, Genet., 19, pp. 199-221 (1994)). HOOK is available from Molecular Simulations, San Diego, Calif.

Instead of proceeding to build an agonist of a PPAR BPM18,708- and/or BPM19,107-binding pocket in a stepwise fashion one fragment or chemical entity at a time as described above, agonist or other PPAR binding compounds may be designed as a whole or “de novo” using either an empty binding pocket or optionally including some portion (s) of a known inhibitor (s). There are many de novo ligand design methods including:
1. LUDI (H. -J. Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.
2. LEGEND (Y. Nishibata et al., Tetrahedron, 47, p. 8985 (1991)). LEGEND is available from Molecular Simulations Incorporated, San Diego, Calif.
3. LeapFrog (available from Tripos Associates, St. Louis, Mo.).
4. SPROUT (V. Gillet et al., “SPROUT: A Program for Structure Generation)”, Comput. Aided MoI. Design, pp. 127-153 (1993)). SPROUT is available from the University of Leeds, UK.

Other molecular modelling techniques may also be employed in accordance with this invention (see, e.g., N. C. Cohen et al., “Molecular Modelling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, Reviews in Computational Chemistry, Vol. 5, K. B. Lipkowitz and D. B. Boyd, Eds., VCH, New York, pp. 337-380 (1994); see also, W. C. Guida, “Software For Structure- Based Drug Design”, Curr. Opin. Struct. Biology, 4, pp. 777-781 (1994)).

The compound can also be selected or designed to possess structural features conferring on the compound an ability to alkylate chemical or biological substrates. In some embodiments the structural features comprise a group (e.g. a tertiary amine—the N carrying the R1 and R2 groups if Formula 1) which is capable of giving rise to a quinone-methide intermediate having potential alkylating activity on chemical or biological substrates. Optionally, the compound is capable of giving rise to such quinone-methide intermediate upon protonation of the tertiary amine and addition of a nucleophile on the H atom of a hydroxyquinoline moiety.

Once a chemical entity has been designed or selected by the above methods, the efficiency with which that chemical entity may bind to the BPM18,708- and/or BPM19,107-binding pocket of, e.g., PPARγ, may be tested and optimized by computational evaluation. For example, an effective BPM18,708- and/or BPM19,107-binding pocket modulator must preferably demonstrate a relatively small difference in energy between its bound and free states (i.e., a small deformation energy of binding). Thus, the most efficient BPM18,708- and/or BPM19,107-binding pocket modulators should preferably be designed with a deformation energy of binding of not greater than about 10 kcal/mole. BPM18,708- and/or BPM19,107-binding pocket inhibitors may interact with the BPM18,708- and/or BPM19,107-binding pocket in more than one conformation that is similar in overall binding energy. In those cases, the deformation energy of binding is taken to be the difference between the energy of the free chemical entity and the average energy of the conformations observed when the inhibitor binds to the protein.

A chemical entity designed or selected as binding to a BPM18,708- and/or BPM19,107-binding pocket of, e.g., PPARγ, may be further computationally optimized so that in its bound state it would preferably lack repulsive electrostatic interaction with the target enzyme and with the surrounding water molecules. Such non-complementary electrostatic interactions include repulsive charge- charge, dipole-dipole and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. ®1995; AMBER, version 4.1 (P. A. Kollman, University of California at San; QUANTA/CHARMM (Accelrys Inc., San Diego, ®2001, 2002); Insight II/Discover (Molecular Simulations, Inc., San Diego, Calif. ®1998; DelPhi (Molecular Simulations, Inc., San Diego, Calif. ®1998; and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art. Other examples of software that can be used to screen and/or model interactions are those used in the examples herein, e.g. Decoys for Docking, AutoDock Vina and Xscore.

Another approach enabled by this invention, is the computational screening of small molecule databases for chemical entities or compounds (e.g. compounds comprising a 8-hydroxyquinoline nucleus, unsubstituted or substituted, linked at the 4 position, though a methylene group, to an N-group; bis-8-hydroxyquinoline compounds; compounds of Formulae I or III) that can bind to the BPM18,708- and/or BPM19,107-binding pocket of, e.g., PPARγ. In this screening, the quality of fit of such entities to the BPM18,708- and/or BPM19,107-binding pocket may be judged either by shape complementarity or by estimated interaction energy (E. C. Meng et al., J. Comp. Chem., 13, pp. 505-524 (1992)).

According to another aspect, the invention provides compounds which associate with the BPM18,708- and/or BPM19,107-binding pocket of, e.g., PPARγ, optionally where the compounds are produced or identified by the method set forth above.

Another particularly useful drug design technique enabled by this invention is iterative drug design. Iterative drug design is a method for optimizing associations between a protein and a compound by determining and evaluating the three-dimensional structures of successive sets of protein/compound complexes.

In iterative drug design, crystals of a series of protein or protein complexes are obtained and then the three-dimensional structure of each crystal is solved.

Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized

In some cases, iterative drug design is carried out by forming successive protein-compound complexes and then crystallizing each new complex. High throughput crystallization assays may be used to find a new crystallization condition or to optimize the original protein or complex crystallization condition for the new complex. Alternatively, a pre-formed protein crystal may be soaked in the presence of an inhibitor, thereby forming a protein/compound complex and obviating the need to crystallize each individual protein/compound complex.

Once a PPAR agonist has been optimally selected or designed, as described herein, substitutions can then be made in some of its atoms or side groups in order to improve or modify its binding properties, again using the information provided by the interaction and specificity templates to identify regions amiable to modification. Such substituted chemical compounds may then be analyzed for efficiency of fit to PPAR polypeptides by the same computer methods described in detail, above.

### Assaying the Ability of the Compounds to Activate PPAR

One a candidate PPAR agonist compound is available (e.g. a compound described herein, a compound designed or identified according to the methods described herein), a quantity of the compound can be produced and assays can be carried out to determine if the compound binds to and/or activates a PPAR or PPAR-like protein. Such assays are well known in the art and are for example as described below as assay method 1 and assay method 2.

### Assay Method 1: PPARγ Receptor Binding Assay:

This assay can be used to, with use of a suitable ligand toward the BPM18,708- and/or BPM19,107-binding pocket, hereafter called the “Start-ligand”, identify chemical entities such as, e.g., low molecular weight compounds, which displace the Start-ligand from the a ligand-binding-domain (LBD) of the PPARγ. Exemplary “Start-ligands” include BPM18,708- and/or BPM19,107, as well as other suitable ligands known in the art. When new ligands are designed, these may serve as new Start-ligands for further drug testing and development.

The method is a ligand binding assay based upon IPA (imaging proximity assay) particles and is based on the assay by Nichols et al. ((1998) Anal. Biochem. 257: 112-119) and ((1998) Anal. Biochem. 263: 126).

The method can be described as follows: The Start-ligand is marked with ³H, while GST-PPARγ-LBD is marked with biotin and the scintillation proximity assay SPA particles are coated with Streptavidin (SA). The receptor is coupled to Glutathiontransferase (GST); GST is a tag that is used to purify the receptor from homogenized cells. When the SPA particles are added they bind to the biotin residues on the GST-PPARγ-LBD. ³H-Start-ligand binds to GST-PPARγ-LBD, and the proximity between the radioactive tritium and the SPA particle results in emission of light from the SPA particles. The amount of light emitted is proportional to the amount of ³H-Start-ligand bound to the binding protein. When a compound that displaces the Start-ligand is present it results in a decrease in the amount of light emitted. Following the binding constant (K_{d}) can be determined Compounds with a Kd value <1 mM is regarded as a “Hit” and can be used further in the drug design process. The Hit is preferably tested also by assay method 2.

### Assay Method 2: A Cell Based Transfection Assay:

With this PPAR response element (PPRE) reporter assay, the selective transactivation of PPARγ in chosen cancer cell line after treatment by small molecule compounds, like ligand Hits from assay 1, can be evaluated. Differences in transactivation between different ligands for a chosen cell lines is screened. The assay is based on the Allred and Kilgore published assay (Allred and Kilgore, (2005) Mol. C. Endoc. 235:21-29).

The PPRE reporter plasmid: A reporter construct, 3XPPRE-TK-pGL3, contains three copies of a PPRE sequence (AGGACAAAGGTCA) upstream of the mTK promoter between the XhoI and Hinólll restriction enzyme sites of the pGL3 basic vector (Promega, Madison, Wis.). BamHI and BgII1 is used to release a 2.2 kb fragment containing the 3×PPRE-mTK-Luciferase. This fragment is ligated into the BamH I receptor site of pRL-TK plasmid (Promega) completing the new reporter which contains both Luciferase and Renilla in a single expression plasmid. Renilla expression is used as a transfection efficiency control.

The transfection assay: Cells are transiently transfected with 5 μg of PPRE reporter plasmid per 12-well plate. Chosen cancer cells are transfected with ESCORT transfection reagent (Sigma-Aldrich) for 4 h. Cells are subsequently treated with the substance to be tested in about micro-molar concentration for 18 h. PPARγ ligand concentrations for each compound used are those shown to be maximally effective following dose response studies. Proper vehicle controls including ethanol, DMSO, and methyl acetate are run for each treatment group. Following treatment, cells are lysed in 50 μl passive lysis buffer and treated according to manufacturer's instructions (Promega dual luciferase assay kit). Luminometry are performed and data are calculated as raw Luciferase Units (RLUs) divided by raw Renilla units. Mean fold induction is obtained by dividing the RLU data from each treatment well by the mean values of the vehicle control appropriate for each treatment. Each set of treatments are performed in replicates of six in three separate experiments. Showing more than 1.05 fold induction change such a compound is regarded as a “hit” and can be used further in the drug design process.

Other methods are readily known by and available to the skilled person in the field. Examples include the use of a known PPAR gamma antagonist such as GW 9668, which can be tested in combination with a candidate PPAR agonist to assess the activity of the candidate PPAR agonist. Also, candidate PPAR agonists can be tested in comparison with a glitazone such as troglitazone in a suitable cellular assay (e.g. HT22 or U87 cell line).

In one embodiment, the invention thus provides a method for identifying a compound which modulates the activity of a PPAR polypeptide, the method comprising: a) contacting said PPAR polypeptide with a compound comprising a 8-hydroxyquinoline nucleus, unsubstituted or substituted, linked at the 4 position though a methylene group to an N-group; a bis-8-hydroxyquinoline compound; or a compound of Formulae I or III, under conditions suitable for binding and/or modulation (e.g. activation) of the activity of said PPAR polypeptide; and b) detecting binding and/or modulation (e.g. activation) of the activity of said PPAR polypeptide by the compound, preferably wherein the compound interacts with an active site or BPM18,708- and/or BPM19,107 binding pocket in said PPAR polypeptide. The PPAR polypeptide may be in any suitable form, e.g. as an isolated polypeptide, in a membrane composition or a polypeptide expressed by a cell. Optionally, the method further comprises assessing whether the compound is capable of alkylating a thiol group on a protein substrate, optionally assessing whether the compound is capable of giving rise to an intermediate having potential alkylating activity, e.g., a quinine-methide intermediate.

### Assaying Pro-Apoptotic Activity

Once a candidate PPAR agonist is obtained, optionally where such candidate has been tested for its ability to bind and/or activate a PPAR, and particularly where such agonist is to be used for treatment of cancer, it can generally be assessed for its ability to interact with, affect the activity of, and/or induce apoptosis or inhibit the proliferation of target cells (e.g. tumor cells). Assessing the compound's ability to induce apoptosis or inhibit the proliferation of target cells can be carried out at any suitable stage of the method, including examples provided herein. This assessment of the ability to induce apoptosis or inhibit proliferation can be useful at one or more of the various steps involved in the identification, production and/or development of an antibody (or other compound) destined for therapeutic use. For example, pro-apoptotic or anti-cell growth/proliferation activity may be assessed in the context of a screening method to identify candidate PPAR agonist compounds, or in methods where an PPAR agonist compound is selected and derivatized. Generally the compound will be known to specifically bind to or interact with a PPAR polypeptide. The step may involve testing a plurality (e.g., a very large number using high throughput screening methods or a smaller number) of test compounds for their pro-apoptotic or anti-cell proliferation activity, or testing a single compound.

Thus, in addition to binding to a PPAR polypeptide, the ability of the compound to induce the apoptosis or inhibit the proliferation of target cells can be assayed. In one embodiment, cells are introduced into plates, e.g., 96-well plates, and exposed to various amounts of the relevant test compound. By adding a vital dye, i.e. one taken up by intact cells, such as AlamarBlue (BioSource International, Camarillo, Calif.), and washing to remove excess dye, the number of viable cells can be measured by virtue of the optical density (the more cells killed or inhibited by the antibody, the lower the optical density). (See, e.g., Connolly et al. (2001) J Pharm Exp Ther 298:25-33, the disclosure of which is herein incorporated by reference in its entirety). Another example is the use of a stain to detect nuclear fragmentation; DAPI (4′,6-diamidino-2-phenylindole) may be used to bind DNA, and fragmentation can then be visualized by detecting fluoresence. To measure cell proliferation or growth, any suitable method such as determining cell number or density, determining the mitotic index, or any other method to determine the number of cells or their position in the cell cycle can be used. Any other suitable in vitro apoptosis assay, assay to measure cell proliferation or survival, or assay to detect cellular activity can equally be used, as can in vivo assays, e.g. administering the antibodies to animal models, e.g., mice, containing target cells, and detecting the effect of the antibody administration on the survival or activity of the target cells over time.

Assays that can be used to determine whether a test compound has pro-apoptotic activity also include assays that determine the compound's effect on components of the cellular apoptotic machinery. For example, as provided in the Examples herein, assays to detect increases or decreases in proteins involved in apoptosis can be used. In one example, a cell (e.g. a cancer cell) is exposed to test compound, and the level or activity of pro-apoptotic and/or anti-apoptotic proteins is measured, for example Bcl-2 protein family members (e.g. Bcl-2, Bax, Bac, Bad, etc.), or caspases (e.g. caspases 3, 7, 8 and/or 9). Compounds will be selected that activate pro-apoptotic protein and/or inhibit anti-apoptotic proteins. Optionally, in view of the selectivity of PPARγ agonists pro-apoptotic activity for cancer cells, a non-cancerous cell can be used as a control. Any test compound, preferably a bis-8-hydroxyquinoline compound or a compound of Formulae I or III, that can detectably stop or reverse tumor growth or kill or stop the proliferation of tumor cells, in vitro or in vivo, can be used in the present methods. Preferably, the test compound is capable of killing or stopping the proliferation (e.g., preventing an increase in the number of target cells, e.g. cancer cells, in vitro or in vivo), and most preferably the test compound can induce the death of such target cells, leading to a decrease in the total number of such cells. In certain embodiments, the antibody is capable of producing a 10%, 20%, 30%, 40%, 50%, 60%, 70%, 80%, 90%, 95%, 96%, 97%, 98%, 99%, or 100% decrease in the number of target cells or in the proliferation of the target cells.

In one embodiment, the test compound is assayed for its ability to activate caspase-3 and/or -7. Exemplary assays are describes in the Examples herein. Any test compound, preferably a bis-8-hydroxyquinoline compound or a compound of Formulae I or III, that can detectably activate or increase the activation of caspase-3 and/or -7, optionally in tumor cells, in vitro or in vivo, can be used in the present methods.

In one embodiment, the invention provides a method for identifying a compound which modulates the activity of a polypeptide involved in apoptosis, the method comprising: a) contacting a cell or a polypeptide involved in apoptosis with a PPAR agonist of the invention under conditions suitable for modulation of the activity of said polypeptide involved in apoptosis; and b) detecting modulation of the activity of said a polypeptide involved in apoptosis by the compound. Optionally, the polypeptide involved in apoptosis is a pro-apoptotic polypeptide (e.g. a caspase, caspase-3) and activation of the pro-apoptotic polypeptide is detected. Optionally, the polypeptide involved in apoptosis is an anti-apoptotic polypeptide and inhibition of the anti-apoptotic polypeptide is detected.

In another embodiment, the invention provides a method for identifying a PPAR agonist compound suitable for use in the treatment of a PPAR-responsive disorder, e.g. a cancer, the method comprising: a) contacting a PPAR polypeptide with a PPAR agonist of the invention, under conditions suitable for binding and/or activation of the activity of said PPAR polypeptide; b) detecting binding and/or modulation (e.g. activation) of the activity of said PPAR polypeptide by the compound, preferably wherein the compound interacts with an active site or BPM18,708- and/or BPM19,107 binding pocket in said PPAR polypeptide; c) contacting a cell or a polypeptide involved in apoptosis with a PPAR agonist of the invention under conditions suitable for modulation of the activity of said polypeptide involved in apoptosis; and d) detecting modulation of the activity of said a polypeptide involved in apoptosis by the compound. Optionally, the polypeptide involved in apoptosis is a pro-apoptotic polypeptide (e.g. a caspase) and activation of the pro-apoptotic polypeptide is detected. Optionally, the polypeptide involved in apoptosis is an anti-apoptotic polypeptide and inhibition of the anti-apoptotic polypeptide is detected.

In another embodiment, the present invention provides a method for producing a PPAR agonist compound suitable for use in the treatment of a PPAR-responsive disorder, the method comprising the following steps: a) providing a PPAR agonist compound of the invention; b) testing the ability of the compound to bind to induce apoptosis or inhibit the proliferation of a substantial number of target cells; and c) selecting and/or producing the compound if it is determined to be capable of inducing apoptosis or inhibiting the proliferation of a target cell. In any of the present methods, a “substantial number” can mean e.g., 30%, 40%, 50%, preferably 60%, 70%, 80%, 90% or a higher percentage of the cells. In a further embodiment, the method further comprises steps d) contacting a PPAR polypeptide with PPAR agonist compound under conditions suitable for binding and/or activation of the activity of said PPAR polypeptide; and e) testing the ability of the compound to bind and/or modulate (e.g. activation) of the activity of said PPAR polypeptide, and selecting and/or producing the compound if it is determined to be capable of modulate (e.g. activation) of the activity of said PPAR polypeptide.

In any of the assays, the PPAR agonist compound may be a compound comprising an 8-hydroxyquinoline nucleus, unsubstituted or substituted, linked at the 4 position though a methylene group to an N-group; a bis-8-hydroxyquinoline compound; or a compound of Formulae I or III. In the assays described herein, the PPAR polypeptide or the polypeptide involved in apoptosis may be in any suitable form, e.g. as an isolated polypeptide or a polypeptide expressed by a cell.

### Functional Assays

Once a compound is identified or obtained it will generally be assayed in one or more functional assays. Functional assays to detect activity in therapeutic applications (e.g. disease models) are well known in the art, including the disease models provided in the Examples section herein. For example, PPAR agonists have been reported to have angiostatic properties; the compounds can be assessed in an in vitro or in vivo in a non-human animal model for their ability to inhibit angiogenesis. In another embodiment, compounds can be assessed for their ability to inhibit tumor cell migration or tumor growth or metastasis. In another embodiment, compounds can be assessed for their ability to induce differentiation of a cell (e.g. cancer cell, pre-adipocytes into mature adipocytes, to cause insulin-sensitization, to cause a biological effect useful in the treatment of, e.g. weight disorders, lipid disorders, metabolic disorders, cardiovascular disease, inflammatory or autoimmune diseases, neurodegenerative disorders, coagulation disorders, gastrointestinal disorders, genitourinary disorders, ophthalmic disorders, infections neuropathic or inflammatory pain, infertility or age-related macular degeneration.

In one example, a functional assay for anti-tumor activity is an in vitro assay using human tumor cells lines. Anti-tumor activity has been demonstrated in the present Examples in the human pancreatic tumor cell line BxPC3 (ATCC, CRL-1687), available from ATCC, Manassas, Va. Cells are cultured at 37° C. in Falcon culture dishes, incubated at 5% CO₂ in a suitable culture medium available from Invitrogen Corp. Anti-proliferative activity is determined by an MTT assay (bromide of (3-[4,5-dimethylthiazolium-yl]diphenyltetrazolium)). According to one standard protocol, absorbance is measured at 570 nm (see Dumont et al. (2007) Neoplasia 9: 766-776.

Once a compound is obtained, and optionally tested in a functional assay, a quantity of such compound will generally be produced and can be formulated for pharmaceutical use.

### Pharmaceutical Compositions

Once a PPAR agonist is identified or obtained and optionally modified or optimized, a quantity of the agonist can be produced and formulated for use as a pharmaceutical. A further embodiment of the present invention provides a pharmaceutical composition comprising a PPAR agonist according to the invention. The present invention provides pharmaceutical compositions comprising the PPAR agonist, in association with one or more pharmaceutically acceptable carriers or excipients. In one embodiment the pharmaceutical composition comprises a prophylactically or therapeutically effective amount of the active pharmaceutical ingredient (API) in admixture with pharmaceutically acceptable excipients wherein the API comprises a detectable amount of the PPAR agonist of the present invention. In a second embodiment the pharmaceutical composition comprises a prophylactically or therapeutically effective amount of the API in admixture with pharmaceutically acceptable excipients wherein the API comprises about 5% to about 100% (e.g. 50% to about 100%, 25% to about 100%) by weight of the PPAR agonist of the present invention.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration or for administration by inhalation or insufflation. Formulation of the compositions according to the invention can conveniently be effected by methods known from the art, for example, as described in Remington's Pharmaceutical Sciences, 18^{th} ed., Mack Publishing, Easton, Pa. (1990).

Dosages of the present invention, when used for the purpose of activating or stimulating immune cells, will generally range between about 0.01 mg per kg of body weight per day (mg/kg/day) to about 100 mg/kg/day, preferably 1 to 50 mg/kg/day, and most preferably 5 to 30 mg/kg/day.

Advantageously, the PPAR agonist may be administered in a single daily dose, or the total daily dosage may be administered in divided doses of two, three or four times daily. Furthermore, the PPAR agonist can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The composition, shape, and type of dosage forms of the invention will typically vary depending on their use. For example, a dosage form used in the acute treatment of a disease or disorder may contain larger amounts of the active ingredient than a dosage form used in the chronic treatment of the same disease or disorder. Similarly, a parenteral dosage form may contain smaller amounts of the active ingredient than an oral dosage form used to treat the same disease or disorder.

In the methods of the present invention, the PPAR agonist can form the API, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

### Oral Dosage Forms

Pharmaceutical compositions of the invention that are suitable for oral administration can be presented as discrete dosage forms, such as, but not limited to, tablets (including without limitation scored or coated tablets), pills, granules, lozenges, caplets, capsules, chewable tablets, powder packets, cachets, troches, wafers, aerosol sprays, or liquids, such as but not limited to syrups, elixirs, solutions or suspensions in an aqueous liquid, a non-aqueous liquid, an oil-in-water emulsion, or a water-in-oil emulsion. Such compositions contain a predetermined amount of a pharmaceutically acceptable PPAR agonist salt, and may be prepared by methods of pharmacy well known to those skilled in the art. See generally, Remington's Pharmaceutical Sciences, 18^{th} ed., Mack Publishing, Easton, Pa. (1990).

Typical oral dosage forms of the invention are prepared by combining the pharmaceutically acceptable PPAR agonist salt in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, (a) surface stabilizers, (b) dispersion aid, (c) binders, (d) filling agents, (e) lubricating agents, (f) glidants, (g) suspending agents, (h) sweeteners, (i) flavoring agents, (j) preservatives, (k) buffers, (l) wetting agents, (m) disintegrants, (n) effervescent agents, (o) humectants, (p) controlled release agents, (q) absorption accelerators, (r) absorbents, (s) plasticisers.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

In particular, examples of excipients that can be used in oral dosage forms of the invention include, but are not limited to, binders, fillers, disintegrants and lubricants.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL-PH-101, AVICEL-PH-103, Avicel PH 102, Avicel PH 112 and Avicel PH 302 AVICELRC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103 and Starch 19,167 LM.

Examples of fillers suitable for use in the pharmaceutical compositions and dosage forms disclosed herein include, but are not limited to, talc, calcium carbonate (e.g., granules or powder), calcium phosphate, microcrystalline cellulose, powdered cellulose, lactose, dextrates, kaolin, mannitol, silicic acid, sorbitol, sucrose, maltodextrin, starch, pre-gelatinized starch, polymethacrylates, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the invention. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant. Disintegrants that can be used to form pharmaceutical compositions and dosage forms of the invention include, but are not limited to, agar-agar, alginic acid, guar gum, calcium carbonate, microcrystalline cellulose, croscarmellose sodium, carboxymethylcellulose calcium, methylcellulose, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), sodium benzoate, sodium stearylfumarate, zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This invention further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the invention can comprise excipients which are well known in the art and are listed in the USP(XXI)/NF (XVI), which is incorporated herein by reference. In general, lactose-free compositions comprise a pharmaceutically acceptable PPAR agonist salt, a binder/filler, and optionally a lubricant, in pharmaceutically compatible and pharmaceutically acceptable amounts.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formular kits. Examples of suitable packaging include, but are not limited to, hermetically sealed foils, plastics, unit dose containers (e.g., vials) with or without desiccants, blister packs, and strip packs.

### Controlled and Delayed Release Dosage Forms

Pharmaceutically acceptable PPAR agonist salts can be administered by controlled- or delayed-release means. Controlled-release pharmaceutical products have a common goal of improving drug therapy over that achieved by their non-controlled release counterparts. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185. These dosage forms can be used to provide slow or controlled-release of one or more active ingredients using, for example, alginic acid, aliphatic polyesters, bentonite, cellulose acetate, phthalate, carnuba wax, chitosan, ethylcellulose, guar gum, microcrystalline wax, paraffin, polymethacrylates, povidone, xanthan gum, yellow wax, carbomers, hydroxypropylcellulose, hydroxypropylmethylcellulose, methylcellulose, other polymer matrices, gels, permeable membranes, osmotic systems (such as OROSX (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of PPAR agonists and thus effect controlled delivery of the drug. Examples of specific anion exchangers include, but are not limited to Duolite A568 and DuoliteAP143 (Rohm & Haas, Spring House, Pa. USA).

One embodiment of the invention encompasses a unit dosage form which comprises a pharmaceutically acceptable PPAR agonist salt, and one or more pharmaceutically acceptable excipients or diluents, wherein the pharmaceutical composition or dosage form is formulated for controlled-release. Specific dosage forms utilize an osmotic drug delivery system.

### Parenteral Dosage Forms

Parenteral dosage forms can be administered to patients by various routes, including, but not limited to, subcutaneous, intravenous, intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared.

Suitable vehicles that can be used to provide parenteral dosage forms of the invention are well known to those skilled in the art. Examples include, without limitation: sterile water; Water for Injection USP; saline solution; glucose solution; aqueous vehicles such as but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of a PPAR agonist disclosed herein can also be incorporated into the parenteral dosage forms of the invention, including conventional and controlled-release parenteral dosage forms.

### Topical, Transdermal and Mucosal Dosage Forms

Topical dosage forms of the invention include, but are not limited to, creams, lotions, ointments, gels, shampoos, sprays, aerosols, solutions, emulsions, and other forms know to one of skill in the art. See, e.g. Remington's Pharmaceutical Sciences, 18th ed., Mack Publishing, Easton, Pa. (1990); and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure.

Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18th Ed., Mack Publishing, Easton, Pa. (1990).

Transdermal and mucosal dosage forms of the invention include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18^{th} Ed., Mack Publishing, Easton, Pa. (1990); and Introduction to Pharmaceutical Dosage Forms, 4^{th} Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Suitable excipients (e.g., carriers and diluents) and other materials that can be used to provide transdermal and mucosal dosage forms encompassed by this invention are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of a PPAR agonist of the invention. For example, penetration enhancers can be used to assist in delivering the active ingredients to or across the tissue. Suitable penetration enhances include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient (s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient (s) so as to improve delivery. In this regard, stearates can serve as a lipid vehicle for the formulation, as an emulsifying agent or surfactant, and as a delivery-enhancing or penetration-enhancing agent.

### Methods of Treatment

The PPAR agonists of the invention will be administered in a therapeutically or prophylactically effective amount to a patient or individual in order to achieve a specific outcome. Accordingly, the present invention provides methods of using the herein-described PPAR agonists for the treatment or prevention of disorders where PPAR activation is useful and/or required. Such methods comprise the step of administering to a patient a composition comprising a PPAR agonist of this invention. The PPARs have been recognized as suitable targets for a number of different diseases and conditions. Some of those applications are described, for example, in US Patent Application Publication number US 2007/0072904, the disclosure of which is hereby incorporated by reference in its entirety. Additional applications are known and the present compounds can also be used for those diseases and conditions.

Thus, PPAR agonists, such as those described herein by Formulae I or III can be used in the prophylaxis and/or therapeutic treatment of a variety of different diseases and conditions where PPAR (e.g. PPAR-γ) activation is useful and/or required, such as weight disorders (e.g., including, but not limited to, obesity, overweight condition, bulimia, and anorexia nervosa), lipid disorders (e.g., including, but not limited to, hyperlipidemia, dyslipidemia (including associated diabetic dyslipidemia and mixed dyslipidemia), hypoalphalipoproteinemia, hypertriglyceridemia, hypercholesterolemia, and low HDL (high density lipoprotein)), metabolic disorders (e.g., including, but not limited to, Metabolic Syndrome, Type II diabetes mellitus, Type I diabetes, hyperinsulinemia, impaired glucose tolerance, insulin resistance, diabetic complication (e.g., including, but not limited to, neuropathy, nephropathy, retinopathy, diabetic foot ulcer, bladder dysfunction, bowel dysfunction, diaphragmatic dysfunction and cataracts)).

The PPAR agonists can also be used in the prophylaxis and/or therapeutic treatment of cardiovascular disease (e.g., including, but not limited to, hypertension, coronary heart disease, heart failure, congestive heart failure, atherosclerosis, arteriosclerosis, stroke, cerebrovascular disease, myocardial infarction, and peripheral vascular disease).

The PPAR agonists can also be used in the prophylaxis and/or therapeutic treatment of inflammatory diseases (e.g., including, but not limited to, autoimmune diseases (e.g., including, but not limited to, vitiligo, uveitis, optic neuritis, pemphigus foliaceus, pemphigoid, inclusion body myositis, polymyositis, dermatomyositis, scleroderma, Grave's disease, autoimmune diabetes, Hashimoto's disease, chronic graft versus host disease, ankylosing spondylitis, rheumatoid arthritis, inflammatory bowel disease (e.g. ulcerative colitis, Crohn's disease), systemic lupus erythematosis, Sjogren's Syndrome, and multiple sclerosis), diseases involving airway inflammation (e.g., including, but not limited to, asthma and chronic obstructive pulmonary disease), inflammation in other organs (e.g., including, but not limited to, polycystic kidney disease (PKD), polycystic ovary syndrome, pancreatitis, nephritis, and hepatitis), otitis, stomatitis, sinusitis, arteritis, temporal arteritis, giant cell arteritis, and polymyalgia rheumatica), skin disorders (e.g., including, but not limited to, epithelial hyperproliferative diseases (e.g., including, but not limited to, eczema and psoriasis), dermatitis (e.g., including, but not limited to, atopic dermatitis, contact dermatitis, allergic dermatitis and chronic dermatitis), and impaired wound healing)).

The PPAR agonists can also be used in the prophylaxis and/or therapeutic treatment of neurodegenerative disorders (e.g., including, but not limited to, Alzheimer's disease (optionally in patients not expressing the ApoE4 allele, see Risner et al., (2006) Pharmacogenomics J. 6(4):246-54), Parkinson's disease, Huntington's disease, amyotrophic lateral sclerosis, spinal cord injury, and demyelinating disease (e.g., including, but not limited to, acute disseminated encephalomyelitis and Guillain-Bane syndrome)).

The PPAR agonists can also be used in the prophylaxis and/or therapeutic treatment of coagulation disorders (e.g., including, but not limited to, thrombosis), gastrointestinal disorders (e.g., including, but not limited to, gastroesophageal reflux, appendicitis, diverticulitis, gastrointestinal ulcers, ileus, motility disorders and infarction of the large or small intestine), genitourinary disorders (e.g., including, but not limited to, renal insufficiency, erectile dysfunction, urinary incontinence, and neurogenic bladder), ophthalmic disorders (e.g., including, but not limited to, ophthalmic inflammation, conjunctivitis, keratoconjunctivitis, corneal inflammation, dry eye syndrome, macular degeneration, and pathologic neovascularization).

The PPAR agonists can also be used in the prophylaxis and/or therapeutic treatment of infections (e.g., including, but not limited to, viral infections, lyme disease, hepatitis infection, Hepatitis B virus (HBV), hepatitis C virus (HCV), Human immunodeficiency virus (HIV), and Helicobacter pylori) and inflammation associated with infections (e.g., including, but not limited to, encephalitis, meningitis). Optionally, in treatment of infections diseases, beneficial effects in treatment of infectious disease may arise from anti-inflammatory activity and/or other mechanisms of the PPAR agonists. For example, in the treatment of patients infected with HCV, reports such as Dharancy et al., (2009) PPAR Res. Article ID 357204, doi: 10.1155/2009/357214 demonstrate that PPAR agonists can be useful for the treatment of HCV due to their anti-inflammatory properties that can prevent, e.g. liver inflammatory response, liver damage, disruption of lipid and glucose metabolism, hepatocyte fat accumulation and fibrosis.

The PPAR agonists can also be used in the prophylaxis and/or therapeutic treatment of neuropathic or inflammatory pain, pain syndromes (e.g., including, but not limited to, chronic pain syndrome, fibromyalgia), infertility, and cancer (e.g., including, but not limited to, pancreatic cancer, glioblastomas, breast cancer and thyroid cancer), as well as in preventing, reducing, lowering, retarding or suppressing age-related macular degeneration and diabetic retinopathy or a disorder of the group of cellular oxidative stress and/or oxidized LDL formation, cell dysfunction, mitochondrial cell dysfunction, tissue dysfunction and tissue degeneration cardiovascular or adipose tissue (for the latter see e.g. WO2008/134828).

The PPAR agonists can also, particularly in view of their ability to enter the CNS and moreover for their neuroprotective activity, be used in the treatment or prevention of a variety of CNS or psychiatric disease, including but not limited to stroke (e.g. treatment of individuals susceptible to stroke), ischaemia, cerebrovascular injury, schizophrenia, bipolar disorder, depression, anxiety disorders, motor neuron disorders, Parkinson's disease, multiple sclerosis and traumatic brain injury. PPARδ has been associated with schizophrenia (Sun S L et al., (2008) Psychiatr Genet. 18(5):253-254). PPAR agonists have also shown to be useful in neuroprotection in stroke and neurodegenerative diseases (Bordet R et al. (2006) Biochem Soc Trans. 34(Pt 6):1341-1346). The neuroprotective effects of PPAR agonists are believed to be related to their inhibition of inflammation. In each case, treatment can be carried out in individuals susceptible to or suffering from a particular condition. For example, in an individual susceptible to ischemia, the PPAR agonist can be administered just before, or several days (e.g. 3-7 days), before the cerebral ischaemia, or can be administered during the reperfusion period. As has been shown with PPAR agonist fenofibrate, a preventative treatment (14 days in the case of fenofibrate) may reduce susceptibility to and severity of stroke.

The PPAR agonist can also be used in the treatment or prevention of renal diseases, including but not limited to chronic kidney disease. For example Perico et al. (2008) Nature Reviews Drug Discovery 7, 936-953 report that PPAR agonists are effective in treating diabetic nephropathy, e.g. as a renoprotectant in diabetic nephropathy. PPAR agonists reduced urinary albumin excretion and ameliorated glomerular injury.

In certain embodiments a method for treating or preventing a PPAR-responsive condition in a subject comprises determining whether a subject suffers from a PPAR-responsive condition, and upon a positive determination that the subject suffers from a PPAR-responsive condition, administering to the subject an amount of a PPAR agonist of the invention (e.g. a 8-hydroxyquinoline compound) in an amount effective to activate a PPAR polypeptide.

In certain embodiments, the method of stimulating a PPAR response in a subject according to the invention comprises the additional step of detecting PPAR activity in the subject following the administration of a PPAR agonist of this invention. The detection of activity is optionally performed on PPAR-expressing cells obtained from a subject after a period of time following administration of the composition.

In certain embodiments, the PPAR agonists are used in a method to induce the differentiation of cells, e.g. PPAR-expressing cells. In one embodiment, the PPAR agonists can be used to induce differentiation of pre-adipocytes into mature adipocytes (terminal differentiation), optionally causing insulin-sensitization in vivo. In one embodiment, the PPAR agonists can be used to induce differentiation of tumor or other hyperproliferative or non-terminally differentiated cells.

In one embodiment, the PPAR agonists are used to treat a non-cancer condition. In other aspects, any of a large number of types of cancer can be treated or prevented using the present PPAR agonists. Essentially, any cancer (or other condition) that can be treated, slowed in its progression, or prevented, by an increase in the activity of PPAR-agonism and induction of apoptosis can be treated. Examples of cancer types or proliferative diseases that can be treated include carcinoma, including that of the bladder, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma.

In one aspect, the PPAR agonists of the invention are effective in preventing the migration and/or proliferation of tumor metastases, and/or in tumors resistant to treatment with one or more other anti-cancer agents or treatments. In one aspect, the compounds of the invention are administered to a subject having a cancer which has not responded, or progressed or relapsed following treatment with a first line or therapy, e.g. a chemotherapeutic agent), or who has a drug resistant cancer. In one aspect, the compounds of the invention are administered to a subject having a metastatic cancer. In Examples provided by the inventors, compounds of the invention (e.g. of Formula III) were active in pancreatic cancer, and in a variety of human tumor cell lines or cell lines known to be resistant to standard anti-tumor agents, including oral squamous carcinomas (represented by cell line KB), colon carcinomas (represented by HTC116, HT29, HTC15 and LoVo cells), mammary carcinomas (represented by MCF7 and MCF7R cells), pulmonary carcinomas (represented by A549 cells), prostate cancers (represented by PC3 cells), glioblastomas (represented by SF268 cells), ovarian adenocarcinomas (represented by SK-OV-3 cells), hepatocarcinomas (represented by HepG2 cells), lymphoclastomas (represented by HLDO and K562 cells), and on non-tumoral cells lines from monkey kidney (represented by VERO cells), or gliomas (represented by U373, Hs683, T98G cells).

In one embodiment for treating cancer, a sample of cancer cells, optionally PPAR-expressing cells, is obtained from the patient prior to the administration of PPAR agonist, and the ability of one or more of the PPAR agonist to activate PPAR activity or induce the apoptosis the cells will be assessed on a portion of that sample. Following the assessment of activation or apoptotic potential, the patient's cell can be activated in vivo, in which the PPAR agonist (in an appropriate pharmaceutical formulation) is directly administered to the patient.

The PPAR agonists of the invention can be used as single agents in therapy or prevention. Alternatively, the PPAR agonists can be used in combination with another therapeutic or prophylactic agent. In one example, a subject is treated with a PPAR agonist of the invention in combination with a second therapeutic agent; the second therapeutic agent may be any agent useful in the treatment of the particular disease condition. In one example, the second therapeutic agent is a PPAR agonist (e.g. a PPARγ agonist, see Sargeant et al. (2004) Br J. Pharmacol. 143(8): 933-937 for rosiglitazone or other agonists in the treatment of cancer). As used herein, the terms “conjoint”, “in combination” or “combination therapy”, used interchangeably, refer to the situation where two or more agents (e.g. an antigen-binding compound of the invention and a chemotherapeutic agent) affect the treatment or prevention of the same disease. The use of the terms “conjoint”, “in combination” or “combination therapy” do not restrict the order in which the agents are administered to a subject with the disease. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a disease.

When cancer is being treated using the PPAR agonist, the method of the present invention may comprise the additional step of administering to said patient another anti-cancer compound or subjecting the patient to another therapeutic approach. For solid tumor treatment, for example, the administration of a composition of the present invention may be used in combination with classical approaches, such as surgery, radiotherapy, chemotherapy, and the like. The invention therefore provides combined therapies in which the present PPAR agonists are used simultaneously with, before, or after surgery or radiation treatment; or are administered to patients with, before, or after conventional chemotherapeutic, radiotherapeutic or anti-angiogenic agents, or targeted immunotoxins or coaguligands.

Examples of other anti-cancer compounds that can be used in combination therapy with the PPAR agonists of the invention include cytokines, e.g. IL-1α IL-1β, IL-2, IL-3, IL-4, IL-5, IL-6, IL-7, IL-8, IL-9, IL-10, IL-11, IL-12, IL-13, IL-15, IL-21, TGF-beta, GM-CSF, M-CSF, G-CSF, TNF-alpha, TNF-beta, LAF, TCGF, BCGF, TRF, BAF, BDG, MP, LIF, OSM, TMF, PDGF, IFN-alpha, IFN-beta, IFN-gamma. Agents can include anti-tumor antibodies such as anti-CD20 antibodies (e.g. rituximab), anti-HER2 antibodies (e.g. Herceptin), etc. A variety of hormonal therapy and chemotherapeutic agents may be used in the combined treatment methods disclosed herein, including any of the agents set forth above as useful in combination compositions of this invention. Exemplary chemotherapeutic agents contemplated as exemplary include alkylating agents, antimetabolites, cytotoxic antibiotics, nucleoside analogues, vinca alkaloids, for example adriamycin, dactinomycin, mitomycin, caminomycin, daunomycin, doxorubicin, tamoxifen, taxol, taxotere, vincristine, vinblastine, vinorelbine, etoposide (VP-16), 5-fluorouracil (5FU), cytosine arabinoside, cyclophosphamide, thiotepa, methotrexate, camptothecin, actinomycin-D, mitomycin C, cisplatin (CDDP), oxaliplatin, gemcitabine, folic acid, aminopterin, combretastatin(s) and derivatives and prodrugs thereof. Also contemplated are protein kinase inhibitors including for example inhibitors or VEGFR1, VEGFR2, PDGFR, mTOR, C-KIT and/or one or more raf kinases (e.g. Raf-a, raf-b and/or raf-c), e.g. Sutent, antibodies such as Avastin (bevacizumab). Preferred hormonal agents include for example LHRH agonists such as leuprorelin, goserelin, triptorelin, and buserelin; anti-estrogens such as tamoxifen and toremifene; anti-androgens such as flutamide, nilutamide, cyproterone and bicalutamide; aromatase inhibitors such as anastrozole, exemestane, letrozole and fadrozole; and progestagens such as medroxy, chlormadinone and megestrol. When PPAR agonists of the invention are used to treat carcinomas, particularly pancreatic cancer, they can be used advantageously in combination with 5FU, gemcitabine or cisplatin. When PPAR agonists of the invention are used to treat glioblastoma, they can be used advantageously in combination with temozolomide (Temodar™).

As with cancer, the methods of the invention of treating or preventing an infectious disease can comprise the addition step of administering to said subject another agent useful for the treatment of infection. Infection medicaments include but are not limited to anti-bacterial agents, anti-viral agents, anti-fungal agents and anti-parasitic agents. Anti-viral agents are of particular interest, and include compounds that prevent infection of cells by viruses or replication of the virus within the cell. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogs), maturation of new virus proteins (e.g. protease inhibitors), and budding and release of the virus. Preferred nucleoside analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine). Another class of anti-viral agents that may be administered with the PPAR-agonist of this invention includes cytokines such as interferons, such as alpha and beta-interferon. Also possible is immunoglobulin therapy, including normal immune globulin therapy and hyper-immune globulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Examples of hyper-immune globulins include zoster immune globulin (useful for the prevention of varicella in immuno-compromised children and neonates), human rabies immune globulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncytial virus infections).

As with cancer, the methods of the invention of treating or preventing a PPAR-responsive condition or disease (e.g. weight disorders, lipid disorders, metabolic disorders, cardiovascular disease, inflammatory or autoimmune diseases, neurodegenerative disorders, coagulation disorders, gastrointestinal disorders, genitourinary disorders, ophthalmic disorders, infections neuropathic or inflammatory pain, infertility) can comprise the addition step of administering to said subject another agent useful for the treatment of the particular disorder. Examples of such agents useful in inflammatory or autoimmune disorders include but are not limited to an immunomodulatory agent, a hormonal agent, an anti-inflammation drug, a steroid, an immune system suppressor, a corticosteroid, an antibiotic, an anti-viral or an adjunct compound. Examples of agents useful in weight, lipid or metabolic disorders include but are not limited to exogenous insulin or drugs that increase the secretion of endogenous insulin, including for example incretin mimetics or glucagon-like peptide-1 analogies such as exenatide, liraglutide, sulfonylurea derivatives such as glimepiride, tolazamide, or gliclazide, amylin analogues, DPP-4 inhibitors, meglitinides, or biguanides (e.g. metformin). Examples of agents that can be used in combination with PPAR agonists of the invention include agents which modulate the renin-angiotensin-aldosterone system (e.g. angiotenisin receptor blockers, angiotensin II receptor antagonists, losartan, irbesartan, olmesartan, candesartan and valsartan and the like, e.g. for treatment or prevention of diabetic nephropathy

PPAR agonists of the invention can further be used advantageously in combination with other agents known to have synergistic or additive activity with PPAR agonists. Examples include RXR agonists (see e.g. US patent publication no. US 20080255206, for the treatment of metabolic and cardiovascular diseases. In another aspect, PPAR agonists of the invention can be used in combination with other PPAR agonists, such as for example glitazone compounds.

Particularly where cancer is being treated, the PPAR agonists of the invention having pro-apoptotic activity can further be used advantageously in combination with other agents known to have synergistic or additive activity with such pro-apoptotic agents. Such combinations will be particularly advantageous in the treatment of solid and/or metastatic tumors. For example, PPAR agonists of the invention activate caspase-3, thereby have potential activity in sensitizing cancer cells to treatment with another agent capable of inducing death or preferably apoptosis of cancer cells; examples include chemotherapeutic agents (e.g. agents that interfere with DNA replication, mitosis and chromosomal segregation, and agents that disrupt the synthesis and fidelity of polynucleotide precursors. For example, agents include alkylating agents, antimetabolites, cytotoxic antibiotics, vinca alkaloids, tyrosine kinase inhibitors, metalloproteinase and COX-2 inhibitors; cyclophosphamide, cisplatin, docetaxel, paclitaxel, erlotinib, irinotecan, bevacizumab or gemcitabine; in pancreatic cancer gemcitabine or cisplatin; in CNS cancers, Temodar™).

When the PPAR agonists are administered to a patient with another agent, the two components may be administered either as separately formulated compositions (i.e., as a multiple dosage form), or as a single composition (such as the combination single dosage forms described above containing a PPAR agonist of this invention and another therapeutic agent).

### Administration

Experience in the field of PPAR-gamma activators has shown that they are typically effective when administered once or twice daily, including when administered orally. See, for example Actos™ (pioglitazone), (Physicians Desk Reference, (2001) 55′″ edition, p 3175 or Avandia™ (rosiglitazone), Physicians Desk Reference, (2001) 55th edition, p 15 3875. However, other suitable administrations regimens can be used as well; e.g. WO03/055485 describes dosing regimens comprising less than once daily administration, which compared to daily or twice daily administration of PPAR-gamma activator, may result in no significant decrease in efficacy.

Accordingly, in one aspect, the PPAR agonists of the invention are administered daily. In another embodiment, a dosing regimen may comprise at least one period in which the frequency of dosing is less than daily, for example every other day, or which comprises at least one gap of at least 1 day between preceding and following administration of PPAR agonist, on which gap there is no administration of the PPAR agonist. For example, the dosing regimens of this invention include regimens comprising 5 days with and 2 days without administration of the PPAR agonist, or 12 with and 2 without, etc. Exemplary dosing regimens are those that comprise periods of every other day or every third day or twice weekly dosing, or that comprise one or two or three consecutive days without administration. When the PPAR agonists is used in combination with administration of either exogenous insulin or drugs that increase the secretion of endogenous insulin, the insulin or additional drug can be administered with a dosing regimen that is the same as or different from the PPAR agonist regimen. As used herein “dosing” and “administration” are intended to be identical.

Optionally, when treating a cancer or a non-cancer condition, the PPAR agonist is used at less that the maximal tolerated dose (MTD), so long as the desired action is achieved (e.g. PPAR activation, apoptosis, caspase activation, alkylation of substrates). Optionally the PPAR agonist is administered to a human in a dose that is between about 1 and about 100%, optionally between about 25 and 100%, optionally between about 25 and 75%, of the (single administration) MTD. MTD can be determined as described in WO2004/050096, the disclosure of which is incorporated herein by reference.

As discussed, specific dosage ranges suitable for the administration of the PPAR agonists are disclosed herein. Optionally, the PPAR agonists is administered to a human subject at a dose comprised between 0.01 and 100 mg/kg, optionally between 0.1 and 50 mg/kg, optionally between 15 and 45 mg/kg (the general dose range used for glitazones), optionally between 1 and 30 mg/kg, optionally between 5 and 30 mg/kg, optionally between 1 and 15 mg/kg. In another aspect, the dose may be comprised between 50 and 1000 mg/m² per day, optionally between 500 and 900 mg/m² per day; optionally between 90-1800 mg/patient per day, or between 900-1800 mg/patient per day.

In one aspect the present invention relates especially to the treatment or prevention of a PPAR-responsive disease characterized in that a PPAR agonist is administered more than once per week, to a human in a dose that is calculated according to the formula (A):

(single dose (mg/kg)=(1 to 50))*d)*w (A)

where d is the number of days of treatment, optionally consecutive or non-consecutive, within one week, and where w is the number of weeks of treatment. More preferably, the treatment dose is calculated according to the formula B,

(single dose (mg/kg)=(5 to 30))*d)*w (B)

or according to the formula C,

(single dose (mg/kg)=(less than 15, or about 1, 2, 3, 4, 5, 6, 7, 8, 9 or 10))*d)*w (C)

where, in each of formulae A to C, d is about 1 to about 7, preferably about 2 to 5, optionally wherein successive treatments within one week are separated by no more than two intervening days, preferably no more than 72 hours, 48 hours or 24 hours, and where w is the number of weeks of treatment, preferably where w is 2, 3, 4, 5, 6, 8, 12, 15 or greater.

### EXAMPLES

Further aspects and advantages of this invention are disclosed in the following experimental section, which should be regarded as illustrative and not limiting the scope of this application.

### Example 1

### Synthesis of Compounds

Compounds were prepared according the general following procedure described in PCT/FR2008/000399 and French patent application no. 0807426 filed on 23 Dec. 2008, the disclosures of which are incorporated herein by reference. Briefly, one equivalent of 5-(chloromethyl)quinolin-8-ol (see Burkhalter et al., 1961. J. Org. chem, 26, 4078) was heated overnight with 2.5 equivalents of the selected amine in dry acetonitrile, in the presence of 5 equivalents of potassium carbonate. The reaction was followed by TLC. The solution is evaporated, and the solid residue is directly purified by column chromatography using solvent gradients (EtOac/Hexane). Unless indicated otherwise, starting materials and reagents were obtained from commercial suppliers and used without purification. Tetrahydrofuran (THF) was distilled on sodium-cetobenzyphenon immediately before use. CH₂Cl₂ was distilled on P₂O₅ immediately before use. NMR spectra were recorded at 250 MHz for 1 hour on a Brucker AC-250 spectrometer. Chemical displacements were expressed in δ units (ppm) with respect to TMS (tetramethylsilane). Electrospray mass spectra were measured on a Waters Micromass ZMD spectrometer by direct injection of the sample solubilised in CH₃CN. Thin layer chromatography (TLC) for analytical purposes were carried or on silica plates of 0.2 mm thickness. Thin layer chromatography (TLC) for preparative purposes were carried or on silica plates of 1 mm thickness.

A solution of 5-chloromethylquinolin-8-ol dihydrochloride (300 mg, 1.3 mmol) and specific amines (1 eqmmol) methyl acetate (10 ml) is stirred at 50° C. overnight. It is cooled to 0° C. and filtered; the filter cake is washed with cold ethyl acetate (5 ml). The filtrate is concentrated in vacuo, diluted in diethyl ether (5 ml) and centrifuged. The etheral phase is removed and the obtained solid is washed two more times by centrifugation with diethyl ether at 0° C. to give the desired compounds as a green powder;

### (1) BPM19,107 5,5′-(4-(methyl)benzylazanediyl)bis(methylene)diquinolin-8-ol

1HNMR (CDCl3) d 2.33 (s, 3H), 3.44 (s, 2H), 3.81 (s, 4H), 6.95e7.17 (m, 8H), 7.36e7.45 (m, 2H), 7.88e7.98 (m, 2H), 8.68e8.76 (m, 2H); ¹³C NMR (CDCl₃) d 20.9, 56.1, 58.0, 108.4, 120.6, 125.1, 127.3, 128.5, 129.4, 129.4, 134.1, 135.6, 136.5, 138.3, 147.2, 151.5; ESI-MS m/z ¼ 436 ([M+H]+, 100%).

### (2) BPM18,725 5,5′-(benzylazanediyl)bis(methylene)diquinolin-8-ol

1H NMR (CDCl3) d 3.66 (s, 2H), 3.99 (s, 4H), 7.20e7.35 (m, 6H), 7.42e7.45 (m, 3H), 7.55e7.58 (m, 2H), 8.03e8.07 (m, 2H), 8.89 (s, 2H); ¹³C NMR (CDCl3) d 56.4, 58.5, 108.8, 120.8, 125.2, 127.2, 127.6, 128.1, 129.7, 129.8, 134.3, 138.6, 139.0, 147.5, 151.8; ESI-MS m/z ¼ 422 ([M]pH]p, 100%).

### (3) BPM19,178 5,5-(2-(Trifluoromethyl)benzylazanediyl)bis(methylene)diquinolin-8-ol

1H NMR (s, 4H), 7.07e7.12 (m, 2H), 7.17 (m, 2H), 7.41 (m, 2H), 7.51 (m, 4H), 7.86 (m, 2H), 8.73 (m, 2H); ¹³C NMR (CDCl3) d 30.9, 56.6, 58.1, 76.5, 77.0, 77.5, 108.7, 121.0, 124.7, 127.4, 129.8, 129.9, 133.9, 138.6, 147.6, 152.0; ESI-MS m/z ¼ 490 ([M+H]+, 100%.

### (4) BPM19,219 5,5′-cyclohexylmethylazanediyl-bis-[(methylene)di(quinolin-8-ol)]

One equivalent of 5-(chloromethyl)quinolin-8-ol was heated overnight with 2.5 equivalents of the cyclohexylmethyl amine in dry acetonitrile, in the presence of 5 equivalents of potassium carbonate.

H¹NMR; 1.1 to 1.3 (11H, cyclohexyl protons), 2.0 (2H), 3.9 (4H), 7 to 8.8 (10H aromatic rings protons).

### (5) BPM19,225 4-((bis((8-hydroxyquinolin-5-yl)methyl)amino)-methyl)cyclohexanecarboxylic acid

¹NMR (250 Mhz, CDCl₃): 7.64 (d, 2H, J=7.75 Hz), 7.41-7.39 (m, 2H), 7.22-7.06 (m, 4H), 6.95-6.8 (m, 4H), 3.61 (s, 4H), 3.44 (s, 2H), 1.2-1.8.61 (s, 10H). MS, m/z calcd 471, [M+H]⁺. found, 471.

### (6) BPM18,708 5,5′-(4-(trifluoromethyl)benzylazanediyl)bis(methylene)diquinolin-8-ol

Yield 90%.

¹NMR (250 MHz, CDCl₃): 8.73 (m, 2H), 7.86 (m, 2H), 7.51 (m, 2H), 7.41 (m, 2H), 7.17 (m, 2H), 7.12-7.07 (m, 4H), 3.84 (br s, 4H), 3.55 (s, 2H). MS, m/z (C₂₈H₂₂F₃N₃O₃): calculated 506.1 [M+H]⁺. measured, 506.1. Anal. (C₂₈H₂₂F₃N₃O₃) C, H, N.

### (7) BPM 19,189 5,5′-(3-(trifluoromethyl)benzylazanediyl)bis(methylene)diquinolin-8-ol

Yield 58%.

¹NMR (250 MHz, CDCl₃): 8.76 (m, 2H), 7.93 (m, 2H), 7.52-7.48 (m, 2H), 7.43-7.40 (m, 2H), 7.31-7.29 (m, 2H), 7.15-7.08 (m, 4H), 3.85 (s, 4H), 3.55 (s, 2H). MS, m/z (C₂₈H₂₂F₃N₃O₂): calculated 490.5 [M+H]⁺. measured, 490.5. Anal. (C₂₈H₂₂F₃N₃O₂) C, H, N.

### (8) BPM18,201 5,5′-(3,5-bis(trifluorométhyl)benzylazanediyl)bis(méthylène)diquinoléin-8-ol

The compound was synthesized from 3,5-(trifluoromethyl)benzylamine and 5-(chloromethyl)quinolin-8-ol according to the general method described herein.

Yield 62%. ¹HNMR (250 MHz, CDCl₃): 8.76-8.74 (m, 2H), 7.96-7.92 (m, 2H), 7.64 (s, 1H), 7.43-7.38 (m, 4H), 7.19-7.13 (m, 2H), 7.08-7.05 (m, 2H), 3.89 (br s, 4H), 3.61 (s, 2H). MS, m/z (C₂₉H₂₁F₆N₃O₂): calculated 558.5 [M+H]⁺. measured, 557.5. Anal. (C₂₉H₂₁F₆N₃O₂) C, H, N.

### (9) BPM19,205 5,5′-(4-(trifluoromethoxy)benzylazanediyl)bis(methylene)diquinolin-8-ol

The compound was synthesized from (4-(trifluoromethoxy)phenyl)methanamine and 5-(chloromethyl)quinolin-8-ol according to the general method described herein.

Yield 90%. ¹H NMR (250 MHz, CDCl₃): 8.73 (m, 2H), 7.86 (m, 2H), 7.51 (m, 2H), 7.41 (m, 2H), 7.17 (m, 2H), 7.12-7.07 (m, 4H), 3.84 (br s, 4H), 3.55 (s, 2H). MS, m/z (C₂₈H₂₂F₃N₃O₃): calculated 506.1 [M+H]⁺. measured, 506.1. Anal. (C₂₈H₂₂F₃N₃O₃) C, H, N.

(10) BPM 19,200 5,5′-(3-iodobenzylazanediyl)bis(methylene)diquinolin-8-ol

The compound was synthesized from (3-iodophenyl)methanamine and 5-(chloromethyl)quinolin-8-ol according to the general method described herein. The product was purified by chromatography (SiO₂CH₂Cl₂/MeOH 95:5 at 80:20).

Yield 50%. ¹H NMR (250 MHz, MeOD): 8.79 (m, 2H), 8.13 (m, 2H), 7.61-6.92 (m, 10H), 4.02 (s, 2H), 3.72 (br s, 4H). Anal. (C₂₇H₂₂IN₃O₂) C, H, N.

(11) BPM 18,202 5,5′-(thiophen-2-ylmethylazanediyl)bis(methylene)diquinolin-8-ol

The compound was synthesized from (4-(trifluoromethoxy)phenyl)methanamine and 5-(chloromethyl)quinolin-8-ol according to the general method described herein. The product was filtered and washed with CH₃CN (2×20 mL) and ether (40 mL). The solvent was concentrated in a vacuum.

Yield: 95%. ¹H NMR (250 MHz, MeOD): 8.79 (m, 2H), 8.13 (m, 2H), 7.43 (m, 3H), 7.23 (m, 2H), 7.01 (m, 4H), 3.86 (br s, 4H), 3.76 (s, 2H). MS, m/z (C₂₅H₂₁N₃O₂S): calculated 428.1 [M+H]⁺. measured, 428.1. Anal. (C₂₅H₂₁N₃O₂S)C, H, N.

### (12) BPM19,197 5,5′-((tetrahydrofuran-2-yl)methylazanediyl)bis(methylene)diquinolin-8-ol

The compound was synthesized from (tetrahydrofuran-2-yl)methanamine and 5-(chloromethyl)quinolin-8-ol according to the general method described herein.

Yield 90%. ¹H NMR (250 MHz, MeOD): 8.79 (m, 2H), 8.64 (d, 1H, J=1.5 Hz), 8.12 (d, 1H, J=1.5 Hz), 7.35-6.90 (m, 6H), 4.01-3.99 (m, 3H), 3.76-3.65 (m, 4H), 2.76-2.47 (m, 2H), 1.99-1.18 (m, 4H). MS, m/z (C₂₅H₂₅N₃O₃): calculated 416.2 [M+H]⁺. measured, 416.2. Anal. (C₂₅H₂₅N₃O₃) C, H, N.

### (13) BPM19,216 5,5′-((1-methyl-1H-pyrrol-2-yl)methylazanediyl)bis(methylene)diquinolin-8-ol

The compound was synthesized from (1H-pyrrol-2-yl)methanamine and 5-(chloromethyl)quinolin-8-ol according to the general method described herein. The product was purified by chromatography (SiO₂, CH₂Cl₂/MeOH 95:5 at 80:20).

Yield 86%. ¹H NMR (250 MHz, MeOD): 8.83 (dd, 2H, J=1.5 HzF, 1.5 Hz), 7.80 (m, 2H), 7.62 (m, 2H), 7.34 (m, 2H), 7.20 (m, 2H), 6.81 (d, 1H, J=0.75 Hz), 6.40 (d, 1H, J=0.75 Hz), 6.01 (d, 1H, J=0.75 Hz), 3.86 (br s, 4H), 3.76 (s, 3H), 3.41 (s, 2H). MS, m/z (C₂₆H₂₄N₄O₂): calculated 426.7 [M+H]⁺. measured, 426.8. Anal. (C₂₆H₂₄N₄O₂) C, H, N.

### (14) BPM 19,193 5,5′-(2-(pyridin-2-yl)ethylazanediyl)bis(methylene)diquinolin-8-ol)

The compound was synthesized from 2-(pyridin-2-yl)ethanamine and 5-(chloromethyl)quinolin-8-ol according to the general method described herein. The product was purified by chromatography (SiO₂, CH₂Cl₂/MeOH 95:5 at 80:20). Yield 13%.

¹H NMR (250 MHz, MeOD): 8.79 (m, 2H), 8.22 (m, 2H), 7.90 (m, 4H), 7.51-6.98 (m, 6H), 3.88 (br s, 4H), 2.95 (m, 4H). Anal. (C₂₇H₂₄N₄O₂) C, H, N.

### (15) BPM19,214 5,5′-(2-(pyrrolidin-1-yl)ethylazanediyl)bis(methylene)diquinolin-8-ol)

The compound was synthesized from 2-(pyrrolidin-1-yl)ethanamine and 5-(chloromethyl)quinolin-8-ol according to the general method described herein. The product was purified by chromatography (SiO₂, CH₂Cl₂/MeOH 95:5 at 80:20). Yield 26%.

¹H NMR (250 MHz, MeOD): 8.61 (m, 2H), 8.20-8.06 (m, 2H), 7.40-6.90 (m, 6H), 3.78 (br s, 4H), 2.50 (s, 4H), 2.38 (m, 4H), 2.21 (s, 2H), 1.56 (s, 2H). Anal. (C₂₆H₂₈N₄O₂) C, H, N.

### (16) BPM 19,213 5,5′-(4-carboxymethylcyclohexylazanediyl)bis(methylene)diquinolin-8-ol

The compound was synthesized from 4-(aminomethyl)cyclohexanecarboxylic acid and 5-(chloromethyl)quinolin-8-ol according to the general method described herein. The product was purified by chromatography (SiO₂, CH₂Cl₂/MeOH 95:5 at 80:20).

Yield 95%.

¹H NMR (250 MHz, CDCl₃): 8.60-8.65 (m, 2H), 8.23-8.01 (m, 2H), 7.26-7.41 (m, 2H), 7.15-6.98 (m, 4H), 3.71 (br s, 4H), 2.18-2.12 (m, 2H), 2.00-1.92 (m, 1H), 1.75-1.68 (m, 2H), 1.57-1.50 (m, 3H), 1.24-1.18 (m, 2H), 0.36-0.21 (m, 2H). MS, m/z (C₂₈H₂₉N₃O₄): calculated 472.5 [M+H]⁺. measured, 472.5. Anal. (C₂₈H₂₉N₃O₄) C, H, N.

The compounds of Table 1 were prepared, as described below:

TABLE 1

| Structure | Compound | Ra | Rb | Rc |
|---|---|---|---|---|
| 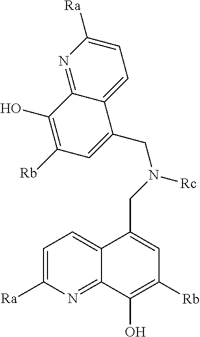 | 17 18 19 20 | H H H CH₃ | (2-méthylvinyl) allyl benzyl H | 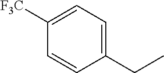 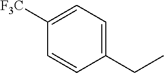 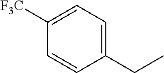 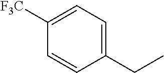 |

For the following compound (17), a mixture of 0.01 mol of 8-hydroxy-5-chloromethylquinoline and allyl bromide are heated under reflux overnight in the presence of potassium carbonate, in acetone. The derivative 8-allyloxy 5-chloromethyl quinoline is thus obtained. The latter compound is directly heated at 160° C. and cooled. The residue obtained after addition of ether is washed successively in alkaline and then acid conditions. The residue is then purified by chromatography. The derivative 7-allyl 8-hydroxy 5-chloromethyl quinoline is thus obtained. This derivative is isomerized to form its homologue in alkaline conditions in methanol by heating to 180° C. for 12 hours. The compound 7-(2-methylvinyl) 8-hydroxy-5-chloromethyl quinoline is thus obtained. The latter intermediate is then brought into the presence of the corresponding primarily amines

### (17) 5,5′((trifluorométhyl)benzylazanediyl)bis(méthylène)di[7-(2-méthylvinyl)quinoléin-8-ol]

H¹NMR (250 MHz, CDCl₃): 8.76-8.74 (m, 2H), 7.96-7.92 (m, 2H), 7.64 (s, 1H), 7.43-7.38 (m, 4H), 7.19-7.13 (m, 2H), 7.08-7.05 (m, 2H), 6.46 (m-1H), 6.92 (d, 1H) 3.89 (br s, 4H), 3.61 (s, 2H). 1.98 (d, 3H)

Compound (18) is obtained by addition of de 4-trifluorobenzylamine in the presence of the derivative 7-allyl 8-hydroxy 5-chloromethyl quinoline previously described.

### (18) 5,5′(trifluorométhyl)benzylazanediyl)bis(méthylène)di[(7-allyl)quinolin-8-ol] (R1=4-CF₃), R2=H, R3=alkyl)

H¹NMR (250 MHz, CDCl₃): 8.76-8.74 (m, 2H), 7.96-7.92 (m, 2H), 7.64 (s, 1H), 7.43-7.38 (m, 4H), 7.19-7.13 (m, 2H), 7.08-7.05 (m, 2H), 6.46 (m-1H), 6.92 (d, 1H), 5.11 (2H), 6.08 (M, 1H) 3.64 (d, 2H) 3.89 (br s, 4H), 3.61 (s, 2H). 1.98 (d, 3H).

### (19) 5,5′(trifluoromethyl)benzylazanediyl)bis(methylene)di[7-benzyl)quinolin-8-ol)

Compound (19) is obtained by condensation of 4-trifluorobenzylamine in the presence of the derivative 7-benzyl-5-chloromethyl-8-hydroxyquinoline.

Yield 56% H¹NMR (250 MHz, CDCl₃): 8.76-8.74 (m, 2H), 7.96-7.92 (m, 2H), 7.64 (s, 1H), 7.43-7.38 (m, 4H), 724, (m, 5H) 7.19-7.13 (m, 4H), 7.08-7.05 (m, 2H), 3.89 (br s, 4H), 3.61 (s, 2H).

### (20) 5,5′-(3,5-bis(trifluoromethyl)benzylazanediyl)bis(methylene)diquinaldine-8-ol

Compound (20) is obtained by condensation of 5-Chloromethyl 8-hydroxyquinaldine with the respective amines, for example with 4-trifluorobenzylamine the desired product is obtained.

Yield 55% H¹NMR (250 MHz, CDCl₃): 8.76-8.74 (m, 2H), 7.96-7.92 (m, 2H), 7.64 (s, 1H), 7.43-7.38 (m, 4H), 724, (m, 5H) 7.19-7.13 (m, 4H), 7.08-7.05 (m, 2H), 3.89 (br s, 4H), 3.61 (s, 2H). 2.8 (3H).

### (21) BPM19,905 5,5′-(propargyl azanediyl)-bis(methylene)diquinolin-8-ol

Yield 30%, white powder, Rf EtOac/hexane 1/1=0.25;

NMR ¹H, 2.35 (1H), 3.2 (2H), 3.9 (4H), 7 to 8.8 (10H aromatic rings protons).

### (22) BPM 19,900 5,5′-(allylazanediyl)-bis(methylene)diquinolin-8-ol

Yield 45%, white powder, Rf EtOac/hexane 1/1=0.28;

¹H NMR 23.3 (2H) 3.9 (4H), 5-5.2 (2H), 6 (1H), 7 to 8.8 (10H aromatic rings protons).

### (23 BPM19,904 5,5′-(isopropyl azanediyl)-bis(methylene)diquinolin-8-ol

NMR ¹H; 0.9 (6H) 2.0 (1H), 3.9 (4H), 7 to 8.8 (10H aromatic rings protons).

### (24) BPM19,899 5,5′-(2,3-dihydro-1H-inden-1-ylazanediyl)-bis(methylene)diquinolin-8-ol

Yield 38%, white powder;

¹H NMR 2.4 (2H), 2.9 (2H) 3.5 (4H) 4.0 (1H), 5.0 (2H), 7.3 to 8.1 (12H aromatic protons), 9.1 2H.

### (25) BPM19,897 5,5′-(benzyloxy azanediyl)-bis(methylene)diquinolin-8-ol

### (26) BPM 19,886 5,5′-(benzyhydryl azanediyl)-bis(methylene)diquinolin-8-ol

### (27) 5,5′-(4-methylbenzylazane diyl)bis(methylene) bis(2-methylquinolin-8-ol)

Yield 52%, white powder, Rf 0.23 EtOAc/Hexane 1/1);

¹H NMR 2.3 (s, 3H), 2.6 (6H), 3.4 (s, 2H), 3.8 (4H), 6.8 to 7.8 (12H aromatic protons).

### (28) BPM19,876 5,5′-(4-trifluoromethylbenzylazanediyl)bis(methylene)bis(2-methylquinolin-8-ol)

Yield 55%, white powder, Rf 0.34 EtoAc/Hexane 1/1;

¹H NMR 2.6 (s, 6H), 3.4 (s, 2H), 3.8 (4H), 6.8 to 7.8 (12H aromatic protons).

### (29) BPM11,208 5,5′-(3,5-ditrifluoromethylbenzylazanediyl)bis(methylene)bis(2-methylquinolin-8-ol)

Yield 62%. ¹HNMR (250 MHz, CDCl₃): 8.76-8.74 (m, 2H), 7.96-7.92 (m, 2H), 7.64 (s, 1H), 7.43-7.38 (m, 4H), 7.19-7.13 (m, 2H), 7.08-7.05 (m, 2H), 3.89 (br s, 4H), 3.61 (s, 2H). 2.6 (6H).

### (30) BPM10,208 5,5′-(thiophene-2-ylmethyl azanediyl)bis(methylene)bis(2-methylquinolin-8-ol)

Yield 42%, white powder;

¹H NMR: 250 MHz CDCl₃ 2.6 (6H), 3.2 (2H), 3.9 (4H), 4.6 to 5.5 3H thiophenyl, 7.2 to 8.1 8H aromatic protons from heterocycle.

### (31) BPM19,902 5,5′-(4-ethoxy-1-(4-hydroxyphenyl)-4-oxobutan-2-ylazanediyl)-bis(methylene)diquinolin-8-ol

Yield 57%, greenish powder;

¹H NMR 0.9, (3H); 2.9 (2H), 3.8 (1H); 3.9 (2H), 5.5 (3H); 6.4 to 8.8 (14H aromatic protons).

Starting from analogues described PCT/FR2008/000399 and French patent application no. 0807426 filed on 23 Dec. 2008, compound 5,5′-(4-trifluoromethylbenzyl azanediyl)-bis(methylene)diquinolin-8-ol was dissolved in acetic acid in the presence of Iodine. The reaction mixture was kept at 50° C. overnight. The desired compound (BPM19,887) was recovered and purified by column chromatography.

### (32) BPM19,887 5,5′-(4-(trifluoromethyl)benzylazanediyl)bis(7-iodoquinolin-8-ol)

Yield 25% yellow powder.

Compound BPM19,888 (33) was obtained through the same methodology as BPM19,887 (32) but starting with 5,5′-(4-methylbenzyl azanediyl)-bis(methylene)diquinolin-8-ol.

### (33) BPM19,888 5,5′-(4-methylbenzylazanediyl)bis(methylene)bis(7-iodoquinolin-8-ol)

Yield 27%, yellow powder.

The suitable amine (2.87 mmol) was added to a stirred solution of 5-chloromethylquinolin-8-ol hydrochloride (5.74 mmol) and K₂CO₃ (8.5 mmol) in CH₃CN (20 mL), and the resulting mixture was heated at 50° C. for 24 h. The reaction was monitored by TLC. The mixture was cooled to 0° C. and filtrated; the filter cake was washed with cold CH₃CN (10 mL). The residue was purified by silica gel column chromatography (CH₂Cl₂/MeOH 95:5) as eluent) to give the required compounds 34 to 36.

### (34) BPM18,726; (35) BPM19,167; and (36) BPM19,191

Compounds 5-((benzylamino)methyl)quinolin-8-ol dihydrochloride (34, BPM18,726), 5-((4-methylbenzylamino)methyl)quinolin-8-ol dihydrochloride (35, BPM19,167), and 5-((4-(trifluoromethyl)benzylamino)methyl)quinolin-8-ol (36, BPM19,191). were prepared following the previously reported method described by Moret et al. ((2008) Eur J Med. Chem. 44:558-567).

### (37) BPM19,114 5-((3-(trifluoromethyl)benzylamino)methyl)quinolin-8-ol

0.76 g of 3-(trifluoromethyl)benzylamine, 1g of 5-(chloromethyl)quinolin-8-ol and 1g of K₂CO₃ were added in 20 mL of CH3CN. The reaction was heated at 50° C. for one day. The product was filtrated and washed by acetonitrile (2×20 mL) and ether (40 mL). The solvent was concentrated under vacuum. Product was purified by chromatography (SiO₂ from DCM/MeOH 95:5 to DCM/MeOH 80:20). Yield 28%. mp=127-129° C. ¹H NMR (250 MHz, MeOD): 8.86 (m, 1H), 8.52-8.49 (m, 1H), 7.42-7.38 (m, 2H), 7.35-7.23 (m, 2H), 7.19-7.14 (m, 2H), 7.02-6.99 (m, 1H), 4.75 (s, 2H), 4.21 (s, 2H). MS, m/z (C₁₈H₁₅F₃N₂O): calcd 333.2 [M+H]⁺. found, 333.2. Anal. (C₁₈H₁₅F₃N₂O) C, H, N.

### (38) BPM19,230 5-((dibenzylamino)methyl)quinolin-8-ol

0.5 g of 5-(aminomethyl)quinolin-8-ol, 0.76 g of K₂CO₃ and 0.72 g of (chloromethyl)benzene were added in 20 mL of CH3CN. The reaction was heated at 50° C. for one day. The product was filtrated and washed by acetonitrile (2×20 mL) and ethyl acetate (40 mL). The solvent is concentrated under vacuum. Product purified by chromatography (SiO₂ from DCM/MeOH 95:5). Yield 75%. mp=142-143° C. ¹H NMR (250 MHz, MeOD): 8.79 (d, 2H, J=1 Hz), 8.39 (d, 1H, J=1 Hz), 7.47-7.01 (m, 13H), 3.82 (br s, 4H), 3.67 (s, 2H). Anal. (C₂₄H₂₂N₂O) C, H, N.

For the preparation of the Boc protected analogues 39-41, Boc₂O (0.58 mmol) was added to a stirred solution of the suitable mono 8-hydroxyquinoline derivatives (0.58 mmol) in CH₂Cl₂ (10 mL). The solution was stirred over night at room temperature then washed with water (4 mL) and brine (2 mL). It was dried over MgSO₄ then concentrated in vaccuo and purified by chromatography eluting with cyclohexane-CH₂Cl₂ (1:1) to give the required compounds 39-41.

### (39) BPM19,192 (8-hydroxyquinolin-5-yl)methyl(4-(trifluoromethyl)benzyl)carbamate

Compound (39) was prepared following the previously reported method described by Moret et al (2008).

### (40) BPM19,132 Tert-butyl-(8-hydroxyquinolin-5-yl)methyl(3-(trifluoromethyl)benzyl)carbamate

Compound (40) was synthesized from compound (37) following the general procedure for the preparation of Boc protected analogues above. Yield 27%. mp=114-116° C. ¹H NMR (250 MHz, CDCl₃): 8.78 (m, 1H), 8.56-8.52 (m, 1H), 7.49-7.34 (m, 2H), 7.37-7.30 (m, 2H), 7.26-7.21 (m, 2H), 7.09-7.06 (m, 1H), 4.82 (s, 2H), 4.28 (s, 2H), 1.95-1.55 (m, 9H). MS, m/z (C₂₃H₂₃F₃N₂O₃): calcd 433.4 [M+H]⁺; found, 433.4. Anal. (C₁₈H₁₅F₃N₂O) C, H, N.

### (41) BPM19,190 Tert-butyl-3,5-bis(trifluoromethyl)benzyl((8-hydroxyquinolin-5-yl)methyl)carbamate

Compound (41) was synthesized from 5-((3,5-difluorobenzylamino)methyl)quinolin-8-ol following the general procedure for the preparation of Boc protected analogues above. Yield 23%. mp=149-152° C. ¹H NMR (250 MHz, CDCl₃): 8.69 (m, 1H), 8.45-8.38 (m, 1H), 7.53-7.47 (m, 1H), 7.40-7.34 (m, 1H), 7.30-7.21 (m, 2H), 7.19-7.16 (m, 1H), 6.98-6.95 (m, 1H), 4.79 (s, 2H), 4.28 (s, 2H), 1.39-1.52 (m, 9H). MS, m/z (C₂₄H₂₂F₆N₂O₃): calcd 501.4 [M+H]⁺. found, 501.4. Anal. (C₂₄H₂₂F₆N₂O₃) C, H, N.

### (42) BPM18,203 N-((8-hydroxyquinolin-5-yl)methyl)-4-methylbenzamide

0.998 g of aminomethyl-8-hydroxyquinoline (5.74 mmol) and 0.883 g of 4 methyl benzoyl chloride (5.74 mmol) were dissolved by 20 mL of CH₃CN. The reaction was heated at 50° C. for two days. Product purified by chromatography (SiO₂ from CH₂Cl₂/MeOH 95:5 to 80:20). Yield 75%. mp=137-139° C. ¹H NMR (250 MHz, CDCl₃, ppm): 8.72 (d, 1H, J=3.75 Hz); 8.31 (d, 1H, J=8.25 Hz); 8.11 (d, 2H, J=8.25 Hz), 7.60 (t, 1H, J=5.25 Hz), 7.47 (d, 1H, J=7.5 Hz), 7.36 (d, 2H, J=7.5 Hz); 7.24 (d, 2H, J=8.25 Hz), 7.12 (d, 1H, J=8.25 Hz), 4.90 (d, 2H), 2.4 (s, 3H). Anal. (C₁₈H₁₆N₂O₂) C, H, N.

### (43) BPM19,218 5,5′,5″-nitrilotris(methylene)triquinolin-8-ol

Compound (43) was synthesized from 5-(aminomethyl)quinolin-8-ol and 5-(chloromethyl)quinolin-8-ol following the general procedure A. Yield 73%. mp=175-178° C. ¹H NMR (250 MHz, MeOD): 8.82-8.79 (m, 3H), 8.59-8.39 (m, 3H), 7.56-7.51 (m, 3H), 7.45-7.42 (m, 3H), 7.05-7.01 (m, 3H), 3.34 (br s, 6H). MS, m/z (C₃₀H₂₄N₄O₃): calcd 489.18 [M+H]⁺. found, 489.1. Anal. (C₃₀H₂₄N₄O₃) C, H, N.

### (44) BPM19,215 5,5′(4-(trifluoromethyl)benzylazanediyl)bis(methylene)diquinolin-8-ol

0.1 g of 5,5′-(4-(trifluoromethyl)benzylazanediyl)bis(methylene)diquinolin-8-ol and 0.018 g of NaH were dissolved in 15 mL of CH₃CN. The reaction was heated at 70° C. for 3 hours. Then the temperature was decreased, and 0.578 g of iodomethane in 7 mL of CH₃CN was added. The reaction was heated again at 70′C for 2 hours. The product was filtrated and washed by CH₂Cl₂ and CH₃CN. Yield 81%. ¹H NMR (250 MHz, CDCl₃): 8.73 (m, 2H), 7.51 (m, 4H), 7.41 (m, 2H), 7.17 (m, 2H), 7.07-7.12 (m, 2H), 4.05 (s, 6H), 3.84 (s, 4H), 3.55 (s, 2H). MS, m/z (C₃₀H₂₆F₃N₃O₂): calcd 518.2 [M+H]⁺. found, 518.2. Anal. (C₃₀H₂₆F₃N₃O₂) C, H, N.

### (45) BPM19,211 5,5′(4-(trifluoromethyl)benzylazanediyl)bis(methylene)diquinolin-8-ol

Compound (45) was prepared following the previously reported method described by Moret et al. (2008).

### Example 2

### Caspase Activation Activity

Compounds BPM18,708 and BPM19,107 were selected as representative analogues and assessed for activity in the induction of caspases.

The experiments were performed by placing BPM18,708 and BPM19,107 in contact with HL60 cells for 48 hours, according to the general protocol described by Margolin et al., J. Biol. Chem. (1997), 272, p. 7223, using standard commercially available diagnostic kits (Calbiochem, USA). Assay reagents used were DEVD for caspases 3/7, IEDT for caspases 8, and LEHD for caspases 9. Moreover, colchicine was used as reference compound that induces this activity of caspases. BPM18,708 and BPM19,107 were tested at three concentrations: 1 μM, 0.1 μM and 0.01 μM.

The results obtained showed that neither BPM18,708 nor BPM19,107 has an effect on the activation of caspases 3/7 at a concentration of 0.01 μM, but they show a strong effect at the other two concentrations (1 μM and 0.1 μM). However, BPM18,708 and BPM19,107 have no effect on the activation of caspases 8 and 9 at the above concentrations.

### Example 3

### PPAR Agonist Activity

Compounds BPM18,708 and BPM19,107 were selected as representative analogues and tested in a PPAR in vitro cellular assay. Thiazolidinediones rosiglitazone and troglitazone were used as reference PPARγ agonists.

For transfection experiments, MCF-7 cells were cultured in six-well plates in 2 ml DMEM:F12 supplemented with 5% FBS. When cells were 50-60% confluent, siRNA duplexes and/or reporter gene constructs were transfected using OligofectAMINE reagent (Invitrogen). According to methods described in previous studies siRNAs were transfected in each well to give a final concentration of 70 nM. Cells were harvested in 48-56 h after transfection by manual scraping in lysis buffer (Promega). Whole cell extracts were frozen in liquid nitrogen for 30 s, vortexed by 30 s, and centrifuged to give lysates cell lysates from this experiment were analyzed for PPARγ protein by Western blot analysis. Relative intensities of the PPARγ protein were compared with a nonspecific band and results of three replicate studies were determined in the various treatment groups.

Whole cell lysates were extracted using Western sampling buffer. Protein samples were heated at 100 C for 5 min, separated on 10% SDS-PAGE and transferred to polyvinylidene difluoride (PVDF) membrane (Amersham Pharmacia Biotech, Piscataway, N.J.). The PVDF membrane was blocked for 1 h and incubated with 1:1000 (cyclin D1), 1:500 (PPARγ), or 1:2000 (Sp1) primary antibody (SantaCruz Biotechnology) for 1 h or with 1:500 (ERa) for 3 h. After vigorous washing for 30 min, 1:2000 secondary antibody was incubated with shaking for 1 h. The membrane was washed for 30 min, incubated with enhanced chemiluminescence substrate (NEN Life Science Products) for 1.0 min, and exposed to Kodak X-Omat AR autoradiography film (Rochester, N.Y.). The membrane was reused and probed with other antibodies as indicated for individual experiments.

Results in transactivation assays showed that both compounds of the series, exemplified by BPM18,708 and BPM19,107 were ligands and agonists of PPARγ.

### Example 4

### Molecular Docking Studies

### Methods

Molecular docking is widely used to predict novel lead compounds for drug discovery. Success depends on the quality of the docking scoring function, among other factors. An imperfect scoring function can mislead by predicting incorrect ligand geometries or by selecting nonbinding molecules over true ligands. These false-positive hits may be considered “decoys”. Although these decoys are frustrating, they potentially provide important tests for a docking algorithm; the more subtle the decoy, the more rigorous the test. Indeed, decoy databases have been used to improve protein structure prediction algorithms and protein-protein docking algorithms. Here, we use two specific molecular docking programs:
decoys for docking ((Decoys for docking. by: Graves A P et al., J Med Chem, Vol. 48, No. 11. pp. 3714-3728.).
AutoDock Vina for molecular docking and virtual screening (O. Trott, A. J. Olson (2009) Journal of Computational Chemistry)
Xscore program was used for its basic functions for computing the binding score of a given ligand molecule (or multiple ligand molecules) to a target protein. All of the parameters needed to run X-Score are assembled in an input parameter file (Wang, R et al., (2002) J. Comput.-Aided Mol. Des. 16: 11-26.).

The docking programs used require the three-dimensional structure of the given protein-ligand complex to calculate its binding constant. The ligand binding domains of PPARα (PDB ID 117G, chain-A), PPARγ (PDB ID 1FM6, chain-B), PPARδ (PDB ID 3GWX, chain-D), and RXRα (PDB ID 1MV9, chain-A) were loaded into the Internal Coordinate Mechanics (ICM) v.3.0 program from the Protein Data Bank (PDB).

Flexible ligand docking was performed on rigid receptors with grid potentials. For each ligand, a stack of the 20 ligand-receptor complexes with low docking energies (ICM docking energy (ICM-DE)) was stored automatically. Each complex was manually examined, and was accepted based on the following criteria: (a) no van der Waals overlap between the ligand and amino acids of the receptor, and (b) oxygen atoms of the ligand should form hydrogen bonds with surrounding polar amino acids in the receptor as observed in the X-ray structures. According to these X-ray crystallographic complexes, following amino acids should form a H-bonding network with the ligand: PPARα—S280, Y314, H440 and Y464; PPARγ—S289, H323, H449 and Y473; PPARδ—H323 and H449; and RXRα—R316 and A327 (main chain oxygen atoms). All-cis DHA was docked into PPARα, PPARγ, PPARδ and RXRα. cis/trans DHA was docked into PPARα and PPARγ, while glitazones were docked into PPARα, PPARγ and PPARδ.

The accepted poses from docking of BPM19,107 and BPM18,708 into PPARα and PPARγ were scored by the two scoring programs. The poses after docking of into BPM19,107 and BPM18,708 RXRα and PPARδ, and the poses after docking of glitazones (reference compounds known to be PPARγ agonists (Scatena et al. PPAR research, 2008, article ID 256251, 10 pages) into PPARγ were scored by ICM-DE.

### Results

Table 2 shows the molecular docking scores by the three docking programs on PPARs. According to the three programs compounds BPM18,708 and BPM19,107 which give binding energies respectively −10 and −9.70 Kcal give the same docking score in Siva and decoys programs. Moreover from binding energy values reported for rosi- and pioglitazone (Sylte et al. J. Mol. Graph. Model., 2008, 27, 217-224), it can be conclude that the analogues BPM18,708 and 19,107 bind PPAR receptors with affinities that are at least as good as that of glitazones. It can also be seen that the compounds appears to bind preferentially to δ and γ receptors over α receptors. We therefore describe molecules predicted to bind various isoforms of PPARs. Molecular docking studies using three different docking programs therefore predicted that compounds BPM18,708 and BPM19,107 are ligands for PPAR receptors, mainly of γ and δ receptors isoforms, supporting the results obtained through the cellular assay experiments. NNC 61-3058 was docked into the binding domain of PPARc crystallized with NNC 61-4424 (pdb code 1KNU).4

TABLE 2

| | gamma | | | delta | | | alpha | |
|---|---|---|---|---|---|---|---|---|
| | 2prg_a | | | 1gwx_a | | | 1K7IA | |
| PPAR | BPM19,107 | BPM18,708 | decoy | BPM19,107 | BPM18,708 | decoy | BPM19,107 | BPM18,108 |
| 1 | −9.6 | −9.9 | −9.8 | −11.2 | −11.9 | −11.5 | −8.5 | −8.6 |
| 2 | −9.7 | −10.1 | −9.8 | −11.2 | −11.9 | −11.5 | −8.6 | −8.6 |
| 3 | −9.7 | −10.1 | −10 | −11.3 | −11.9 | −11.5 | −8.6 | −8.6 |
| 4 | −9.7 | −10 | −10 | −11.3 | −11.9 | −11.4 | −8.4 | −8.2 |
| 5 | −9.7 | −9.8 | −10 | −11.2 | −11.9 | −11.5 | −8.3 | −8.7 |
| | −9.70 | −10.00 | −10.00 | −11.20 | −11.90 | −11.50 | −8.50 | −8.60 |
| convergence | 5 | 3 + 2 | 3 + 2 | 5 | 5 | 5 | 2 + 2 | 3 |
| Xscore_AVE | 7.71 | 7.66 | 7.44 | 7.94 | 8 | 8.08 | 7.82 | 7.83 |
| | −10.52 | −10.45 | −10.15 | −10.83 | −10.91 | −11.02 | −10.68 | −10.69 |
| convergence | 2 | 4 | 3 | 2 | 3 | 5 | 3 | 4 |

### Example 5

### In Vitro Anti-Tumor Activity

Compounds were tested for their ability to induce tumor regression in various carcinoma cells lines as well as specifically for the treatment of neoplasias of the central nervous system, which are classically difficult to treat with conventional therapy. The compounds were tested for their ability to decrease cell proliferation, induce apoptosis and express typical markers of differentiated phenotypes in glioblastoma and astrocytomas cell lines.

### Anti Cancer Activities Concentration (IC₅₀).

Cell growth inhibition was determined by the MTS assay; the number of viable cells were proportional to the extent of formazan production in the presence or absence of drugs. The cytotoxicity index was plotted against the drug concentration which ranged between 0.5 and 10 nM and the value resulting in 50% cytotoxicity was determined (the experiments were performed in triplicate)

Table 3A and B shows results for in vitro anti-cancer activity in KB3 (epitheloid lung cancer) cell lines. “n.d.^{c}” indicates not determined Compounds BPM19,107, BPM18,708 and BPM18,202 emerged as the most active analogues on KB3 cell line with respectively IC50 values of 0.003, 0.001 and 0.002 μM, and have only one order less active than the well known drug docetaxel IC50 value 0.1 nM.

Compounds that met the criteria for activity on KB3 cells were further tested for drug growth inhibitory activity in 5 carcinoma, 6 glioma and 3 melanoma cell lines, shown in Table 4. A549 (human lung adenocarcinoma epithelial), BxPC3 (pancreatic cancer), LoVo (colon cancer), MCF-7 (epithelial breast cancer), PC3 (human prostate cancer), Hs683 (human glioma), T98G (glioblastoma), U373 (human glioblastoma-astrocytoma), U138 MG (human glioma), epithelial-like), SKMEL28 (human melanoma), B16-F10 (mouse melanoma) and RhTP (rhabdomyosarcoma) cell lines were cultured in presence of the drugs for 3 days. The cytotoxic activity is expressed as (IC₅₀). Inhibition of cell proliferation measured by the MTT colorimetric assay. The values reported in Table 4 are means of six distinct values. The standard errors are <5% as compared to the mean values.

As reported in Table 4, compounds 5,5′-(benzylazanediyl)bis(methylene)diquinolin-8-ol (2) (BPM18,725), 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5-(2-(Trifluoromethyl)benzylazanediyl)bis(methylene)diquinolin-8-ol (3) (BPM19,178), 5,5′-(3-(trifluoromethyl)benzylazanediyl)bis(methylene)diquinolin-8-ol (7) (BPM19,189), 5,5′-(3,5-bis(trifluorométhyl)benzylazanediyl)bis(méthylène)diquinoléin-8-ol (8) (BPM18,201), 5,5′-(thiophen-2-ylmethylazanediyl)bis(methylene)diquinolin-8-ol (11) (BPM18,202), 5,5′-(3-iodobenzylazanediyl)bis(methylene)diquinolin-8-ol (10) (BPM19,200), 5,5′-cyclohexylmethylazanediyl-bis-[(methylene)di(quinolin-8-ol)] (4) (BPM19,219) and 4-((bis((8-hydroxyquinolin-5-yl)methyl)amino)-methyl)cyclohexanecarboxylic acid (5) (BPM19,225) showed antitumor activity, including in pancreatic carcinoma cell lines, a relatively resistant cancer. Compounds 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5′-(3-(trifluoromethyl)benzylazanediyl)bis(methylene)diquinolin-8-ol (7) (BPM19,189), and 5,5′-(thiophen-2-ylmethylazanediyl)bis(methylene)diquinolin-8-ol (11) (BPM18,202) were the most active at nanomolar range on glioma cell lines while on carcinoma cell lines the most active compounds were 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5′-(3-(trifluoromethyl)benzylazanediyl)bis(methylene)diquinolin-8-ol (7) (BPM19,189), and 5,5′-(thiophen-2-ylmethylazanediyl)bis(methylene)diquinolin-8-ol (11) (BPM18,202). BxPC3 carcinoma cell lines exhibited a lower sensitivity to analogues 5,5′-(4-(methyl)benzylazanediyl)bis(methylene)diquinolin-8-ol (1) (BPM19,107), and 5,5′-(4-(trifluoromethyl)benzylazanediyl)bis(methylene)diquinolin-8-ol (6) (BPM18,708). Only compounds 5,5′-(3,5-bis(trifluorométhyl)benzylazanediyl)bis(méthylène)diquinoléin-8-ol (8) (BPM18,201) and 5,5′-(thiophen-2-ylmethylazanediyl)bis(methylene)diquinolin-8-ol (11) (BPM18,202) were found to be as active on BxPC3 as on the other carcinoma cell lines shown in Tables 4. Taken together, the data suggest compounds 5,5′-(4-(methyl)benzylazanediyl)bis(methylene)diquinolin-8-ol (1) (BPM19,107) and 5,5′-(4-(trifluoromethyl)benzylazanediyl)bis(methylene)diquinolin-8-ol (6) (BPM18,708) and 5,5′-(thiophen-2-ylmethylazanediyl)bis(methylene)diquinolin-8-ol (11) (BPM18,202) are the most active compounds for treatment across cancers of different types, while other compounds may have preferential potency in one or more tumor types.

TABLE 3A
Structure and cytotoxic activity of 8-hydroxyquinoline analogues

| | | | KB3^{a} |
|---|---|---|---|
| General structure | Compound | R₁ | IC₅₀ (μM) |
| 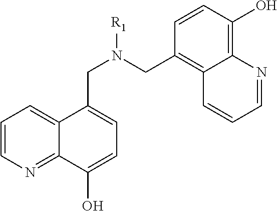 | 18,725 (2) 19,107 (1) 18,708 (6) | 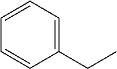 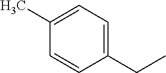 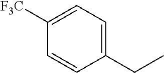 | 0.02 0.003 0.001 |
| | 19,178 (3) | 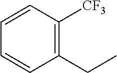 | 0.1 |
| | 19,189 (7) | 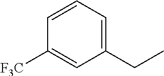 | 2.2 |
| | 18,201 (8) | 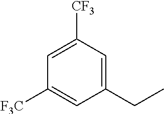 | 1 |
| | 19,205 (9) | 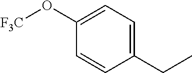 | 0.1-1 |
| | 19,200 (10) | 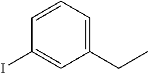 | 1 |
| | 18,202 (11) | 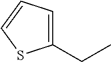 | 0.002 |
| | 19,197 (12) | 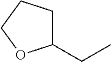 | 10 |
| | 19,216 (13) | 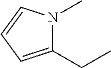 | n.d.^{c} |
| | 19,193 (14) | 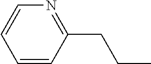 | >10 |
| | 19,214 (15) | 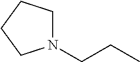 | 1 |
| | 19,213 (16) | 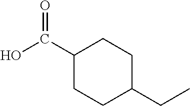 | 1.1 |

TABLE 3B
Structure and cytotoxic activity of 8-hydroxyquinoline analogues

| | | | | | KB3^{a} |
|---|---|---|---|---|---|
| | | | | | IC₅₀ |
| | Compound | R₁ | R₂ | R₃ | (uM) |
| 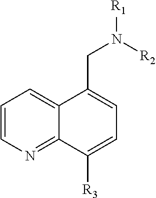 | 18,726 (34) 19,167 (35) 19,191 (36) | 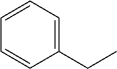 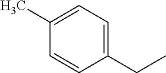 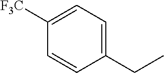 | H H H | OH OH OH | >1 n.d.^{c} 10 |
| | 19,114 (37) | 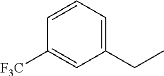 | H | OH | 0.2 |
| | 19,230 (38) | 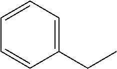 | 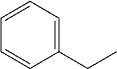 | OH | 1 |
| | 19,192 (39) | 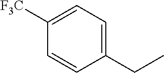 | Boc | OH | 10 |
| | 19,132 (40) | 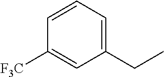 | Boc | OH | 1 |
| | 19,190 (41) | 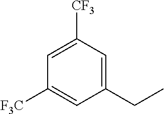 | Boc | OH | 1 |
| | 18,203 (42) | 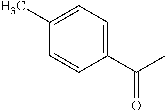 | H | OH | 1 |
| | 19,218 (43) | 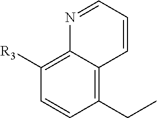 | 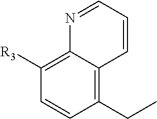 | OH | >10 |
| | 19,215 (44) | 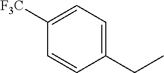 | 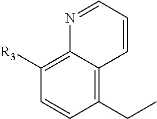 | OMe | >10 |
| | 19,211 (45) | 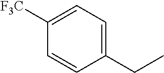 | 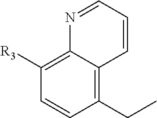 | H | 10 |

TABLE 4

| | | | | | | | | | | | MELANOMA | | | | | |
|---|---|---|---|---|---|---|---|---|---|---|---|---|---|---|---|---|
| | | CARCINOMA | | | | GLIOMA | | | | | | | | | SKMEL- | |
| | | A549 | BxPC3 | LoVo | MCF7 | PC3 | HS683 | T98G | U373 | U138 | GL19 | RhTp | KaKa | Host | 28 | B16F10 |
| (2) | BPM18,725 | 54 | 7442 | 91 | 97 | 258 | 76 | 249 | 357 | 95 | 3448 | 97 | 2434 | 2158 | >10⁴ | 765 |
| (1) | BPM19,107 | 44 | 325 | 43 | 47 | 41 | 51 | 71 | 96 | 81 | 1030 | 84 | 710 | 2455 | 4782 | 88 |
| (6) | BPM18,708 | 8 | 4299 | 10 | 34 | 66 | 10 | 99 | 44 | 49 | 1241 | 81 | 1584 | 97 | >10⁴ | 92 |
| (3) | BPM19,178 | 46 | 274 | 93 | 88 | 179 | 64 | 210 | 78 | 80 | 472 | 87 | 887 | 912 | 6384 | 98 |
| (7) | BPM19,189 | 38 | 167 | 94 | 30 | 37 | 36 | 57 | 65 | 47 | 674 | 69 | 6473 | 679 | >10⁴ | 89 |
| (8) | BPM18,201 | 9 | 79 | 10 | 38 | 71 | 181 | 72 | 96 | 46 | 2143 | 47 | 98 | 146 | >10⁴ | 76 |
| (11) | BPM18,202 | 27 | 89 | 40 | 35 | 44 | 35 | 88 | 85 | 44 | 3451 | 79 | 1757 | 1726 | 4826 | 86 |
| (10) | BPM19,200 | 350 | 3810 | 393 | 374 | 394 | 479 | 446 | 5595 | 430 | >10⁴ | 788 | 7678 | 2976 | 9854 | 888 |
| (4) | BPM19,219 | 369 | 3810 | 357 | 365 | 421 | 584 | 486 | 2573 | 446 | 8970 | 865 | 5937 | 1536 | >10⁴ | 873 |
| (5) | | 661 | 4337 | 422 | 9461 | 993 | 2857 | 9755 | 3726 | >10⁴ | 7604 | >10⁴ | >10⁴ | >10⁴ | >10⁴ | >10⁴ |

### Example 6

### Mechanism of Action

### Rationale for Cytotoxic Potency

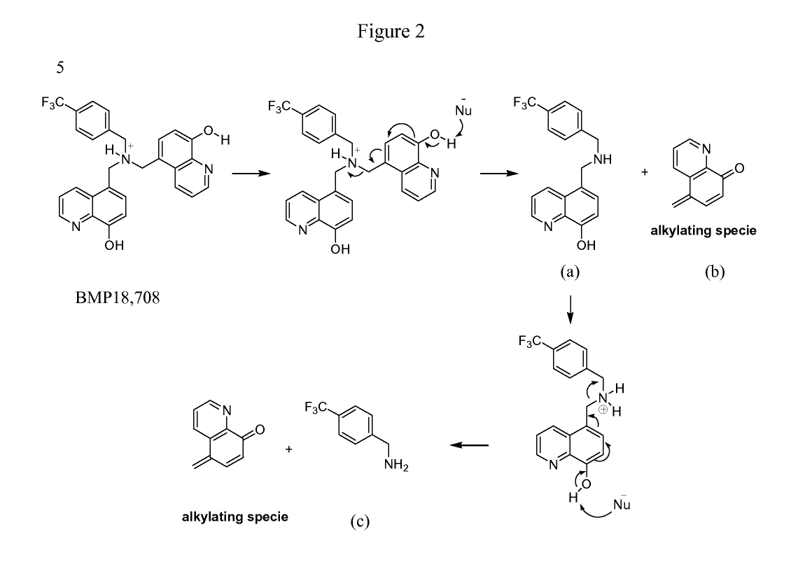

In considering the electronic properties and the chemical reactivity of the N-benzyl 8-hydroxyquinoline, the possibility that this scaffold may behave as an alkylating agent promoter was envisaged. In specific conditions (temperature, solvent, and nucleophile reactants) 8-hydroxyquinoline derivatives can undergo the formation of quinone methide intermediates. Several examples of drugs whose antitumor activities have been attributed to their capacity to generate alkylating intermediates have been reported. In this perspective, we hypothesized the possibility for the N-benzyl 8-hydroxyquinoline derivative BPM18,708 to generate a quinone methide intermediate (compound b) through the pathway underlined in FIG. 2. This proposed mechanism of action requires two steps, a protonation step followed by a nucleophilic attack leading to the generation of the quinone methide intermediate. In order to support this hypothesis of a possible cytotoxic activity through a quinone methide alkylating intermediate, we investigated a structure-cytotoxic activity relationship based on the results obtained against the KB3 cell line reported in Table 3, above.
BPM18,708 was the most active analogue of this series of bis 8-hydroxyquinoline substituted benzyl amines with two 8-hydroxyquinoline moieties.
Compound BPM19,215 in which both hydroxyl groups have been protected by methoxy substituents was, as expected, found to be totally inactive at 10 μM, a feature that confirms the requirement of free hydroxyl groups at the 8 position of the quinolinyl nucleus.
Compound BPM19,211, in which the two 8-hydroxyl groups at the 8 position of the heterocyclic nucleus have been removed cannot undergo the formation of the suggested quinone methide alkylating species, and was found to be 4 orders of magnitude less potent than its corresponding hydroxylated analogue BPM18,708 on the KB3 cell line.
Compounds BPM19,192 (CC₅₀=10 μM) and BPM18,203 (CC₅₀=1 μM) in which the tertiary amine was respectively replaced by a carbamate or amide function, showed cytotoxic effects noticeably reduced. This drop in cytotoxic effect can be attributed to the presence of carbamate or amide functions which are non protonable functions; a feature that once more confirms that a protonation step is mandatory for initiating the formation of the quinone methide alkylating intermediate.

This structure-cytotoxicity correlation is consistent with the proposed quinone methide formation hypothesis; since structural changes favouring or impeding the generation of this intermediate directly influence their cytotoxicity against the KB3 cell line. The formation of the quinone methide intermediate required two major structural conditions: (1) Presence of a protonable function represented by the tertiary amine function. Analogues bearing non-protonable functions, such as BPM19,192 and BPM19,215 were found inactive. Presence of at least one free hydroxyl group at the 8 position of the quinoline nucleus appears to be a favouring feature for promoting activity.

Compounds BPM19,192, BPM18,203, BPM19,211 and BPM19,215, whose chemical structure does not fulfil these two structural requirements are inactive or only weakly active. Such structures do not favour, or may impede, the formation of the quinone methide intermediate. A second quinone methide intermediate could also be generated from (compound a in FIG. 2), after protonation of the newly formed secondary amine leading subsequently to the formation of tri-fluorobenzylamine (compound c) as shown in FIG. 2. This observation is consistent in part with the reduced cytotoxic activity observed for most of the mono-8-hydroxyquinoline N-benzyl analogues series analogues compared to the bis-quinoline analogues, although some mono-8-hydroxyquinoline N-benzyl analogues such as BPM19,114 did retain significant cytotoxic activity. Substitution on the quinoline ring(s) may further impact the formation of the quinone methide intermediate, and it is believed that preferred substitutions will involve groups that are not electron donating.

It can be concluded that structural requirements for optimal activity include the following:

1- presence of two 8-hydroxy quinoline moieties both linked to a methylene amino group;
2- absence of electron donating groups in the 2 position of the quinoline group (presence of such groups are detrimental to biological activity); and
3- presence of a free hydroxyl group at the 8 position of the quinoline nucleus.

The third substituent on the tertiary amine can be varied

### Identification of a Quinone Methide Intermediate

Since relatively stable quinone methide intermediates have been described (Lewis et al., Chem Res Toxicol 1996, 9, 1368-74), and in order to identify the postulated quinone methide intermediate, we used a chemical model similar to that reported by Pande et al. ((1999) J Am Chem Soc, 121, 6773-6779) and Brimble et al. ((2000) Journal of the Chemical Society-Perkin Transactions 1, 317-322) in order to provide evidence that O-(tert-butyldimethylsilyl)-2-bromo-methylphenol can generate an ortho-quinone methide in the presence of fluoride anion. According to this model, it appears that when compound BPM18,708 was submitted to a nucleophilic fluoride anionic attack in a DMF/H₂O solvent system (for 48 h at 50° C.), product analysis by mass spectrometry allowed a clear identification of the presence of the quinone methide intermediate (compound b in FIG. 2) (M/z [M+H]⁺=158) as well as the corresponding expected secondary amine product (compound a in FIG. 2) (M/z [M+H]⁺=333). The structure of compound a (FIG. 2) was confirmed by comparison with a genuine sample synthesized separately as described in Moret et al. ((2008) Eur J Med Chem. 44:558-567) We also identified the presence of 4-trifluoro benzyl amine (compound 28 in FIG. 2) in the reaction mixture products, suggesting that the secondary amine (compound a) could also generate a second quinone methide intermediate, as shown in FIG. 2.

NMR analysis of the reaction product mixture which, besides product b, contained also BPM18,708 (un-reacted starting material) and 4-trifluorobenzyl amine (product c), confirmed the obtained mass spectrometry results. While a peak at δ=4.40 (ppm) corresponding to two methylenyl proton signals of the quinone methide (product b) was clearly identified, this ¹H NMR proton signal was not present in the NMR spectra of BPM18,708 and 4-trifluorobenzyl amine (product c).

### Example 7

### In Vivo Anti-Tumor Activity

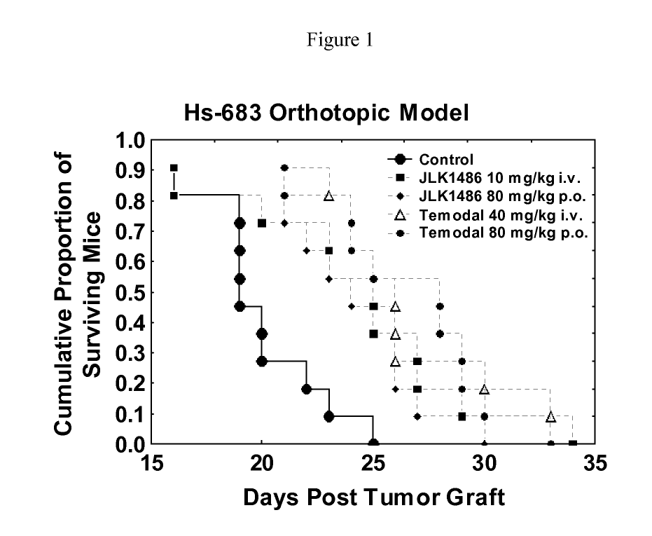

Additional in vivo experiments were carried on nude mice. Briefly, orthotopic grafts of human glioblastoma cell lines were implanted into the brains of nude mice according to the general methodology of Branle et al., (2002) Cancer, 95, 641-655) and Lefranc et al., (2004) Clin. Can. Res., 10, 8250-8265. Results show that BPM18,708 was at least as active as Temodar™. Results are shown in FIG. 1, illustrating the comparative activity of the PPAR agonist BPM18,708 and the reference drug Temodar™ (temozolomide) the only FDA approved drug for glioblastoma grade IV. It can be seen in FIG. 1 that BPM18,708 is at least as active or slightly more active in vivo than Temodar™. Interestingly, Temodar has also been reported to be a caspase 3/7 inducer (Moret et al.(2008)).

Taken together with other Examples herein, results show that compound BPM18,708 is a PPARγ agonist and caspase 3/7 inducer with potent anti-proliferative and anti-migrative properties on various cancer cell lines. This analogue shows promising in vivo activity on orthotopic grafts of human glioblastoma cell lines.

### Example 8

### Neuroprotective Activity

It is known (P. Aoun, 2003 Eur. J. Pharm. 472:65-71) that Peroxisome proliferator-activated receptors (PPARs) are involved in regulating many metabolic and inflammatory processes. The present invention explores the role of PPAR ligands in protecting neuronal cultures from toxic insults. For that purpose, the new PPAR agonists described in Example 1 were screened for their potential role as neuroprotective compounds. Experiments were performed using HT-22, an immortalized mouse hippocampal cell line, and SK-N-SH, a human neuroblastoma cell line. HT-22 was selected because it expresses PPARγ receptors. Cell viability against glutamate, hydrogen peroxide (H₂O₂), and serum deprivation insults was determined using a calcein assay. Troglitazone (a PPARγ reference agonist) as well as compounds BPM18,708, BPM19,107 and other related analogues showed a dose-dependent neuroprotection from glutamate and H₂O₂ insults in HT-22 cells. Additionally, compounds were protective even when administered simultaneously with glutamate, or for up to 8 h postglutamate insult.

### Methods

HT-22, an immortalized mouse hippocampal cell line, and SK-N-SH, a human neuroblastoma cell line, were used. HT-22 cells were obtained from David Schubert (Salk Institute, San Diego, Calif.). The HT-22 line was originally selected from HT-4 cells based on glutamate sensitivity. SK-N-SH cells were obtained from ATCC (Manassas, Va.). HT-22 and SK-N-SH cells were grown to confluency in Dulbecco's modified essential medium (DMEM) and RPMI-1640 media, respectively, and supplemented with 10% charcoal/dextran-treated fetal bovine serum (FBS) and 5 mg/ml gentamicin at 37° C. under 95% air/5% CO₂. HT-22 cells were plated at a density of 50,000 cells/ml (5000 cells/well), and SK-N-SH cells were plated at a density of 120,000-150,000 cells/ml (12,000-15,000 cells/well) in 96-well plates. In most studies, wells were pretreated with PPAR ligands over a wide dose range at various times prior to being subjected to either glutamate, hydrogen peroxide, or serum deprivation insults. In some studies, the insults were applied prior to the addition of the PPAR ligand.

### Results

The following analogues were tested on the HT22 cell line for their neuroprotective effect against glutamate insult and for their anti-inflammatory activity on relevant, models including HT22 cell line.

TABLE 5

| Compound | [C] μM |
|---|---|
| BPM 19,107 (1) | <1 |
| BPM19,189 (7) | <1 |
| BPM18,202 (11) | <1 |
| BPM19,900 (22) | <1 |
| BPM19,902 (31) | <1 |
| BPM19,905 (21) | <1 |
| BPM18,708 (6) | <1 |
| Rosiglitazone | <1 |

As shown in Table 5, the tested compounds exhibit a neuroprotective effect on HT22 cell line after glutamate insults (oxidative glutamate toxicity), as did the reference compound troglitazone. When HT22 cells insulted by addition of various concentrations of glutamate were treated by a known PPAR gamma antagonist (for example GW9662) it is observed that the previous neuroprotective activity found for the BPM compounds were totally or partially abolished, clearly indicating that when PPAR receptors are occupied by a PPAR antagonist, the agonistic effects of the BPM compounds cannot be observed. When the glutamate-insulted HT22 cells were treated with BPM compounds or with any known PPAR agonists such as glitazone series, or by mixture of both agonists, a synergistic activity in neuroprotective activity was observed in some cases, but the cells in all cases had increased survival compared to untreated insulted cells.

### Example 9

### Anti-Infective Activity

Trypanosoma brucei and T. congolense are the causative agents of sleeping sickness in humans and nagana in cattle, respectively. The protozoan parasites live extracellularlyin blood and tissue fluids of mammals and are transmitted by the bite of infected tsetse flies (Glossina spp.). Here the trypanocidal activity against bloodstream forms of T. brucei and T. congolense of bis-8-hydroxy quinoline N-substituted benzylamines derivatives was investigated in vitro, according to methods described in. Steverding et al. (2006) Kinetoplastid Biology and Disease, 5(3): 1-5; Hongmance et al., (2007) Antimicrob. Agents Chemother. 51:1105-1106; and Zhao et al., (2005) Bio-org. Med. Chem.; 13: 3921-3926. Cells were seeded into 96-well tissue culture plates in 200 μl medium containing various concentrations of analogues dissolved in H2O. The controls contained the respective solvent alone. In all experiments, the final solvent concentration was 1% which had no effect on cell growth. To ensure that the cells were in logarithmic growth phase during the entire experiment, they were seeded at an initial density of 1×104 T. brucei/ml, 4×105 T congolense/ml, and 1×105 HL-60/ml, respectively. After 24 h (trypanosomes) or 43 h (HL-60) incubation, 20 μl of the colorimetric viability indicator Alamar Blue® was added to each well. The cells were incubated for a further 24 h (trypanosomes) or 5 h (HL-60) so that the total incubation time was 48 h. Then, the plates were read on a Dynatech MR5000ELISA reader (Denkendorf, Germany),using a test wavelength of 550 nm and a reference wave length of 630 nm. Each test was set up in duplicate and repeated three times. For comparison, the general cytotoxicity of the compounds was assayed with human myeloid leukaemia HL-60 cells. The anti-trypanosomal activities and the general cytotoxicities of the new analogues were evaluated using the Alamar Blue® assay.

The results are shown in Table 6, including the minimum inhibitory concentration (MIC) which is the lowest concentration at which all cells were killed as well as 50% growth inhibition (GI50) which is the inhibitor concentration necessary to reduce the growth rate of the cells by 50%. The anti-trypanosomal activities of some of these compounds approach or are more active than those of commercial drugs used to treat sleeping sickness (suramin: IC50=0.4 μM) and nagana (diminazene aceturate (Berenil®): IC50=0.5 μM) previously determined for bloodstream forms of T. brucei 427-221a and T. congolense STIB910 under identical experimental conditions (Merschjohann et al. (2001) Planta Med 67:623-627) The compounds of the invention can therefore be used to treat infectious disease; furthermore, the compounds can be used in combination with other agents (e.g. non-PPARγ agonists) used for the treatment of infectious disease.

TABLE 6

| Compound | MIC (μM) | GI₅₀ (μM) |
|---|---|---|
| BPM19,107 (1) | 1 | 0.31 ± 0.01 |
| BPM18,708 (6) | 1 | 0.31 ± 0.01 |
| BPM18,201 (8) | 1 | 0.32 ± 0.01 |
| BPM19,205 (9) | 1 | 0.32 ± 0.02 |
| BPM18,202 (11) | 1 | 0.31 ± 0.01 |
| BPM19,876 (28) | 100 | 3.71 ± 0.24 |
| BPM18,725 (2) | 1 | 0.30 ± 0.01 |
| BPM19,702 | 1 | 0.29 ± 0.00 |
| BPM19,167 (35) | 100 | 14.82 ± 2.59 |
| BPM19,178 (3) | 1 | 0.33 ± 0.01 |
| BPM19,189 (7) | 1 | 0.31 ± 0.01 |
| BPM19,197 (12) | 10 | 2.99 ± 0.05 |
| BPM19,216 (13) | 1 | 0.32 ± 0.01 |
| BPM19,218 (43) | 1 | 0.45 ± 0.10 |
| BPM19,219 (4) | 10 | 2.95 ± 0.18 |
| BPM19,226 | 1 | 0.32 ± 0.01 |

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-94. (canceled)

95. A method for treating or preventing a PPAR-responsive condition in a subject, comprising administering to the subject a PPAR agonist that comprises an 8-hydroxyquinoline-methylene-N- group in an amount effective to activate a PPAR polypeptide.

96. The method of claim 95, wherein the PPAR-responsive condition is a condition selected from the group consisting of a weight disorder, a lipid disorder, a metabolic disorder, a cardiovascular disease, an inflammatory or autoimmune disease, a neurodegenerative disorder, stroke, ischaemia, cerebrovascular injury, schizophrenia, bipolar disorder, depression, an anxiety disorder, a motor neuron disorder, Parkinson's disease, multiple sclerosis, traumatic brain injury, a coagulation disorder, a gastrointestinal disorder, a genitourinary disorder, an ophthalmic disorder, an infectious disease, neuropathic or inflammatory pain, infertility, age-related macular degeneration and a renal disorder.

97. The method of claim 95, wherein the PPAR agonist is administered by oral route.

98. The method of claim 95, wherein the PPAR agonist is a compound of Formula I.

99. The method of claim 95, wherein the PPAR agonist comprises a bis-8-hydroxyquinoline nucleus.

100. The method of claim 95, wherein the PPAR agonist is a compound of Formula III.

101. The method of claim 95, wherein the PPAR agonist is a compound selected from the group consisting of 5,5′-(benzyl azanediyl)bis(methylene)diquinolin-8-ol (2) (BPM18,725), 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5-(2-(Trifluoromethyl)benzylazanediyl)bis(methylene)diquinolin-8-ol (3) (BPM19,178), 5,5′-(3-(trifluoromethyl)benzylazanediyl)bis(methylene)diquinolin-8-ol (7) (BPM19,189), 5,5′-(3,5-bis(trifluoromethyl)benzylazanediyl)bis(methylene)diquinolein-8-ol (8) (BPM18,201), 5,5′-(thiophen-2-ylmethylazanediyl)bis(methylene)diquinolin-8-ol (11) (BPM18,202), 5,5′-(3-iodobenzylazanediyl)bis(methylene)diquinolin-8-ol (10) (BPM19,200), 5,5′-cyclohexylmethylazanediyl-bis-[(methylene)di(quinolin-8-ol)] (4) (BPM19,219) and 4-((bis((8-hydroxyquinolin-5-yl)methyl)amino)-methyl)cyclohexanecarboxylic acid (5) (BPM19,225).

102. The method of claim 95, wherein the PPAR agonist is administered daily.

103. The method of claim 95, wherein the PPAR agonist is administered on at least two days per week.

104. The method of claim 95, wherein the effective amount is between 1 and 50 mg/kg.

105. The method of claim 95, wherein the PPAR agonist is administered in combination with a chemotherapeutic agent.

106. The method of claim 95, wherein the PPAR agonist is capable of alkylating a thiol group on a protein substrate.

107. The method of claim 106, wherein the PPAR agonist is capable of giving rise to a quinine-methide intermediate.

108. The method of claim 95, wherein the PPAR agonist comprises an 8-hydroxyquinoline nucleus, unsubstituted or substituted, linked at the 4 position, through a methylene group.

109. The method of claim 95, wherein the PPAR agonist comprises a substitution in a quinoline ring.

110. The method of claim 109, wherein substitution is at position 2 and/or 7 of a quinoline ring.

111. The method of claim 110, wherein the substituent is not an electron donating group.

112. The method of claim 95, wherein the PPAR-responsive condition is a brain tumor, and the PPAR agonist is a compound selected from the group consisting of 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5′-(3-(trifluoromethyl)benzylazanediyl)bis(methylene)diquinolin-8-ol (7) (BPM19,189) and 5,5′-(thiophen-2-ylmethylazanediyl)bis(methylene)diquinolin-8-ol (11) (BPM18,202).

113. The method of claim 95, wherein the PPAR-responsive condition is a carcinoma, and the PPAR agonist is a compound selected from the group consisting of 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18.708), 5,5′-(3,5-bis(trifluoromethyl)benzylazanediyl)bis(methylene)diquinolein-8-ol (8) (BPM18,201) and 5,5′-(thiophen-2-ylmethylazanediyl)bis(methylene)diquinolin-8-ol (11) (BPM18,202).

114. The method of claim 95, wherein the PPAR-responsive condition is a pancreatic cancer and the PPAR agonist is 5,5′-(3,5-bis(trifluoromethyl)benzylazanediyl)bis(methylene)diquinolein-8-ol (8) (BPM18,201) or 5,5′-(thiophen-2-ylmethylazanediyl)bis(methylene)diquinolin-8-ol (11) (BPM18,202).

115. A pharmaceutical composition comprising a PPAR agonist that comprises an 8-hydroxyquinoline-methylene-N- group, or a pharmaceutically acceptable salt thereof, in an amount effective to modulate at least one PPAR-mediated cellular signaling pathway, in combination with a pharmaceutically acceptable carrier.

116. The composition of claim 115, wherein the PPAR agonist is a compound selected from the group consisting of 5,5′-(benzylazanediyl)bis(methylene)diquinolin-8-ol (2) (BPM18,725), 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5-(2-(trifluoromethyl)benzylazanediyl)bis(methylene)diquinolin-8-ol (3) (BPM19,178), 5,5′-(3-(trifluoromethyl)benzylazanediyl)bis(methylene)diquinolin-8-ol (7) (BPM19,189), 5,5′-(3,5-bis(trifluoromethyl)benzylazanediyl)bis(methylene)diquinolein-8-ol (8) (BPM18,201), 5,5′-(thiophen-2-ylmethylazanediyl)bis(methylene)diquinolin-8-ol (11) (BPM18,202), 5,5′-(3-iodobenzylazanediyl)bis(methylene)diquinolin-8-ol (10) (BPM19,200), 5,5′-cyclohexylmethylazanediyl-bis-[methylene)di(quinolin-8-ol)] (4) (BPM19,219) and 4-((bis((8-hydroxyquinolin-5-yl)methyl)amino)-methyl)cyclohexanecarboxylic acid (5) (BPM19,225).

117. A method for treating or preventing a PPAR-responsive condition in a subject, comprising determining whether a subject suffers from a PPAR-responsive condition, and upon a positive determination that the subject suffers from a PPAR-responsive condition, administering to the subject an amount of a PPAR agonist that comprises an 8-hydroxyquinoline-methylene-N- group in an amount effective to activate a PPAR polypeptide.

118. The method of claim 117, wherein the PPAR agonist is a compound of Formula (I), 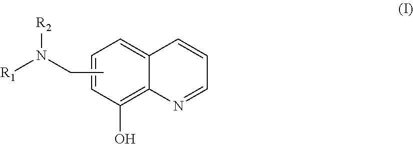
wherein the —CH₂—NR₁R₂ group is in the ortho, meta or para position relative to the —OH group, and in which:
one of the radicals R₁ and R₂ represents a hydrogen atom, a C₁ to C₁₀ alkyl group, a C₂ to C₄ alkenyl or alkynyl group or a 5-methylene-8-hydroxyquinoline group; the other represents a 5-methylene-8-hydroxyquinoline group, a C₃ to C₆ cycloalkyl group, an aryl group, —(CH₂)ₙ-heteroaryl comprising one or more heteroatoms chosen from N, O and S, n being an integer between 0 and 4, a C₄ to C₆ group —(CH₂)ₙ-heterocycloalkyl in which the heteroatom represents N, O or S, n being an integer between 0 and 4, or alkylphenyl in which the alkyl represents C₁ to C₁₀, the cycloalkyl, aryl, heteroaryl, heterocycloalkyl and phenyl group being unsubstituted or substituted with 1 or 2 halogen atoms chosen from F, Br, I and Cl or with —CF₃, a C₁ to C₄ alkyl, COOH, CHO, COOR′ with R′ alkyl in C₁ to C₄;
or one of the radicals R₁ and R₂ represents a group of formula (II) linked to the asymmetric carbon 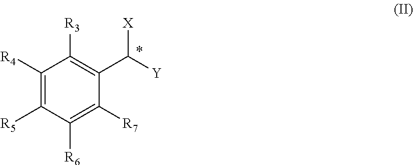
in which R₃, R₄, R₅, R₆ and R₇, independently of each other, represent a hydrogen atom, a C₁ to C₁₀ alkyl group, —CF₃, —NO₂, —NH), an N-5-methylene-8-hydroxyquinoline group, 1 or 2 halogen atoms chosen from F, Br, I and Cl or a group —O—R, R being a C₁ to C₄ alkyl group or —CF₃,
X or Y represents a hydrogen atom, a C₁ to C₁₀ alkyl group, an aryl that is unsubstituted or substituted with a C₁ to C₁₀ alkyl group, —CF₃ or —NO₂,
the other of the radicals R₁ and R₂ representing an H atom, a tert-butoxycarbonyl group (Boc), 5-methylene-8-hydroxyquinoline or —(CH₂)ₙ-phenyl, n being an integer between 1 and 5;
or, when one of the groups R₁ and R₂ is a group Y—N—Y′ in which Y is chosen from the group formed by —(CH₂)ₙ—, n being an integer between 1 and 10, —(CH₂)ₘ-phenyl-(CH₂)ₚ—, the phenyl being unsubstituted or substituted with 1 or 2 halogen atoms chosen from I, F, Br and Cl or with a C₁ to C₁₀ alkyl group, m and p being, respectively, integers between 1 and 4, and in which Y′ is 5-methylene-8-hydroxyquinoline, the other represents a hydrogen atom;
or, when one of the groups R₁ and R₂ represents a group —(CH₂)ₙ-naphthalene, n being an integer between 1 and 10, the naphthalene group being unsubstituted or substituted with one or more groups chosen from C₁ to C₁₀ alkyl groups, —CF₃ and —O—R in which R is a C₁ to C₁₀ alkyl group, the other is chosen from the group consisting of a hydrogen atom, a 5-methylene-8-hydroxyquinoline group and a Boc group;
or R₁ and R₂ form a piperazine in which at least one of the carbon atoms of the ring is substituted with a C₁ to C₆ alkyl group and in which the N atom that is not part of the group —CH₂—NR₁R₂ is substituted with a 5-methylene-8-hydroxyquinoline group;
or R₁ and R₂ form a polyazamacrocycle (cyclam) representing unsubstituted 1,4,8,12-tetraazacyclopentadecane or 1,4,8,11-tetraazacyclotetradecane in which at least one of the N atoms of the ring in position 1, 4 and 8 is, independently, substituted with a Boc group, with a 5-methylene-8-hydroxyquinoline group or with —(CH₂)ₙ-phenyl-(CH₂)ₙ—Z, n being an integer between 1 and 10, in which Z represents one of the N atoms of a 1,4,8,12-tetraazacyclopentadecane or 1,4,8,11-tetraazacyclotetradecane in which the other N atoms of the ring in position 1, 4 and 8 are unsubstituted or are each independently substituted with a Boc group, and pharmaceutically acceptable salts thereof, pharmaceutically acceptable solvates thereof, and enantiomers thereof;
or a compound of Formula (III), 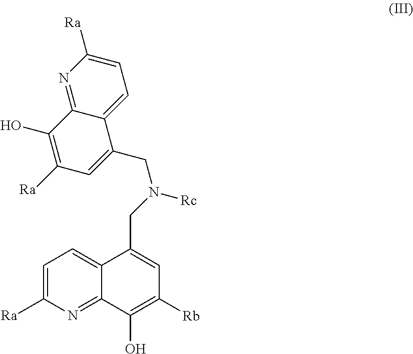
in which:
each Ra represents a C₁-C₆ alkyl group, unsubstituted or substituted with a halogen atom, a —NO₇, NH₂ or —OR group where R is a C₁ to C₄ alkyl group;
each Rb represents an allyl group, a C₂ to C₄ alkenyl or alkynyl group, propargyl or benzyl, the allyl, propargyl or benzyl being unsubstituted or substituted with a halogen atom, a —NO₂, NH₂ or —OR group where R is a C₁ to C₄ alkyl group;
Rc represents a hydrogen atom, a C₁ to C₁₀ alkyl group, a C₂ to C₄ alkenyl or alkynyl group or a 5-methylene-8-hydroxyquinoline group, a C₃ to C₆ cycloalkyl group, an aryl group, a —(CH₂)ₙ-heteroaryl comprising one or several heteroatoms selected from N, O and S, n being an integer between 0 and 4, a C₄ to C₆ —(CH₂)ₙ-heterocycloalkyl group in which the heteroatom represents N, O and S, n being an integer between 0 and 4, or alkylphenyl where the alkyl represents C₁ to C₁₀, the cycloalkyl, aryl, heteroaryl, heterocycloalkyl and phenyl groups being unsubstituted or substituted with one or two groups selected from F, Br, I and Cl, —CF₃, a C₁ to C₄ alkyl, COOH, CHO, COOR′ where R′ is a C₁ to C₄ alkyl group;
or Rc represents a group of formula (II) linked to the asymmetric carbon 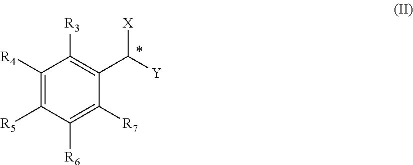
in which R₃, R₄, R₅, R₆ and R₇, independently of each other, represent a hydrogen atom, a C₁ to C₁₀ alkyl group, —CF₃, —NO₂, —NH₂, an N-5-methylene-8-hydroxyquinoline group, 1 or 2 halogen atoms chosen from F, Br, I and Cl or a group —O—R, R being a C₁ to C₄ alkyl group or —CF₃, X or Y represents a hydrogen atom, a C₁ to C₁₀ alkyl group, an aryl that is unsubstituted or substituted with a C₁ to C₁₀ alkyl group, —CF₃ or —NO₂,
or Rc represents a tert-butoxycarbonyl (Boc) group or —(CH₂)ₙ-phenyl, n being an integer between 1 and 5;
or Rc represents a Y—N—Y′ group where Y is selected from the group consisting of —(CH₂)ₙ—, n being an integer between 1 and 10, —(CH₂)ₙ-phenyl-(CH₂)ₚ—, the phenyl being unsubstituted or substituted with 1 or 2 halogen atoms selected from F, Br, I and Cl or with a C₁ to C₁₀ alkyl group, m and p respectively being a number between 1 and 4, and wherein Y′ is 5-methylene-8-hydroxyquinoline; or Rc represents a —(CH₂)ₙ-naphtalene group, n being an integer between 1 and 10, the naphthalene group being unsubstituted or substituted with one or several groups selected from C₁ to C₁₀ alkyl groups, —CF₃ and O—R where R is a C₁ to C₁₀ alkyl group,
and pharmaceutically acceptable salts thereof, pharmaceutically acceptable solvates thereof, and enantiomers thereof.

119. The method of claim 118, wherein:
one of the radicals R₁ and R₂ represents a hydrogen atom, a C₁ to C₆ alkyl group, a C₂ to C₄ alkenyl or alkynyl group or a 5-methylene-8-hydroxyquinoline group;
the other represents a 5-methylene-8-hydroxyquinoline group, an aryl group, —(CH₂)ₙ-heteroaryl comprising one or more heteroatoms chosen from N, O and S, n being an integer between 0 and 4, a C₄ to C₆ group —(CH₁)ₙ-heterocycloalkyl in which the heteroatom represents N, O or S, n being an integer between 0 and 4, or alkylphenyl in which the alkyl represents C₁ to C₆, the phenyl group being unsubstituted or substituted with 1 or 2 halogen atoms chosen from F, Br, I and Cl or with —CF₃;
or one of the radicals R₁ and R₂ represents a group of formula (II) linked to the asymmetric carbon 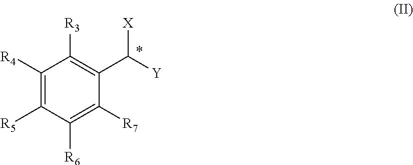
in which R₃, R₄, R₅, R₆ and R₇, independently of each other, represent a hydrogen atom, a C₁ to C₆ alkyl group, —CF₃, —NO₂, —NH₂, an N-5-methylene-8-hydroxyquinoline group, 1 or 2 halogen atoms chosen from F, Br, I and Cl or a group —O—R, R being a C₁ to C₃ alkyl group or —CF₃,
X or Y represents a hydrogen atom, a C₁ to C₆ alkyl group, an aryl that is unsubstituted or substituted with a C₁ to C₆ alkyl group, —CF₃ or —NO₂,
the other of the radicals R₁ and R₂ representing an H atom, a tert-butoxycarbonyl group (Boc), 5-methylene-8-hydroxyquinoline or —(CH₂)ₙ-phenyl, n being an integer between 1 and 5;
or, when one of the groups R₁ and R₂ is a group Y—N—Y′ in which Y is chosen from the group formed by —(CH₂)ₙ—, n being an integer between 1 and 6, —(CH₂)ₘ-phenyl-(CH₂)ₚ—, the phenyl being unsubstituted or substituted with 1 or 2 halogen atoms chosen from I, F, Br and Cl or with a C₁ to C₆ alkyl group, m and p being, respectively, integers between 1 and 4, and in which Y′ is 5-methylene-8-hydroxyquinoline,
the other represents a hydrogen atom;
or, when one of the groups R₁ and R₁₁ represents a group —(CH₂)ₙ-naphthalene, n being an integer between 1 and 6, the naphthalene group being unsubstituted or substituted with one or more groups chosen from C₁ to C₆ alkyl groups, —CF₃ and —O—R in which R is a C₁ to C₆ alkyl group,
the other is chosen from the group consisting of a hydrogen atom, a 5-methylene-8-hydroxyquinoline group and a Boc group;
or R₁ and R₂ form a piperazine in which at least one of the carbon atoms of the ring is substituted with a C₁ to C₄ alkyl group and in which the N atom that is not part of the group —CH₂—NR₁R₂ is substituted with a 5-methyl ene-8-hydroxyquinoline group;
or R₁ and R₂ form a polyazamacrocycle (cyclam) representing unsubstituted 1,4,8,12-tetraazacyclopentadecane or 1,4,8,11-tetraazacyclotetradecane in which at least one of the N atoms of the ring in position 1, 4 and 8 is, independently, substituted with a Boc group, with a 5-methylene-8-hydroxyquinoline group or with —(CH₂)ₙ-phenyl-(CH₂)ₙ—Z, n being an integer between 1 and 6, in which Z represents one of the N atoms of a 1,4,8,12-tetraazacyclopentadecane or 1,4,8,11-tetraazacyclotetradecane in which the other N atoms of the ring in position 1, 4 and 8 are unsubstituted or are each independently substituted with a Boc group, and pharmaceutically acceptable salts thereof, pharmaceutically acceptable solvates thereof, and enantiomers thereof.

120. The method of claim 118, wherein:
one of the radicals R₁ and R₂ represents a hydrogen atom, a C₁ to C₄ alkyl group, a C₂ to C₄ alkenyl or alkynyl group or a 5-methylene-8-hydroxyquinoline group;
the other represents a 5-methylene-8-hydroxyquinoline group, an aryl group, —(CH₂)ₙ-heteroaryl comprising one or more heteroatoms chosen from N, O and S, n being an integer between 0 and 4, a C₄ to C₆ group —(CH₂)ₙ-heterocycloalkyl in which the heteroatom represents N, O or S, n being an integer between 0 and 4, or alkylphenyl in which the alkyl represents C₁ to C₄, the phenyl group being unsubstituted or substituted with 1 or 2 halogen atoms chosen from F, Br, I and Cl or with —CF₃;
or one of the radicals R₁ and R₂ represents a group of formula (II) linked to the asymmetric carbon 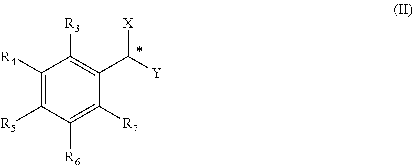
in which one of R₃, R₄, R₅, R₆ and R₇, represents an N-5-methylene-8-hydroxyquinoline group and the others a hydrogen atom,
X or Y represents a hydrogen atom, a C₁ to C₄ alkyl group, an aryl that is unsubstituted or substituted with a C₁ to C₄ alkyl group, —CF₃ or —NO₂,
the other of the radicals R₁ and R₂ representing an. H atom, a tert-butoxycarbonyl group (Boc), or 5-methylene-8-hydroxyquinoline;
or, when one of the groups R₁ and R₂ is a group Y—N—Y′ in which Y is chosen from the group formed by —(CH₂)ₙ—, n being an integer between 1 and 4, —(CH₂)ₘ-phenyl-(CH₂)ₚ—, the phenyl being unsubstituted or substituted with 1 or 2 halogen atoms chosen from I, F, Br and Cl or with a C₁ to C₆ alkyl group, m and p being, respectively, integers between 1 and 4, and in which Y′ is 5-methylene-8-hydroxyquinoline, the other represents a hydrogen atom;
or, when one of the groups R₁ and R₂ represents a group —(CH₂)ₙ-naphthalene, n being an integer between 1 and 6, the naphthalene group being unsubstituted or substituted with one or more groups chosen from C₁ to C₄ alkyl groups, —CF₃ and —O—R in which R is a C₁ to C₄ alkyl group,
the other is chosen from the group consisting of a hydrogen atom, a 5-methylene-8-hydroxyquinoline group and a Boc group;
or R₁ and R₂ form a piperazine in which at least one of the carbon atoms of the ring is substituted with a C₁ to C₃ alkyl group and in which the N atom that is not part of the group —CH₂—NR₁R₂ is substituted with a 5-methylene-8-hydroxyquinoline group;
or R₁ and R₂ form a polyazamacrocycle (cyclam) representing unsubstituted 1,4,8,12-tetraazacyclopentadecane or 1,4,8,11-tetraazacyclotetradecane in which at least one of the N atoms of the ring in position 1, 4 and 8 is, independently, substituted with a Boc group, with a 5-methylene-8-hydroxyquinoline group or with —(CH₂)ₙ-phenyl-(CH₂)ₙ—Z, n being an integer between 1 and 4, in which Z represents one of the N atoms of a 1,4,8,12-tetraazacyclopentadecane or 1,4,8,11-tetraazacyclotetradecane in which the other N atoms of the ring in position 1, 4 and 8 are unsubstituted or are each independently substituted with a Boc group,
and pharmaceutically acceptable salts thereof, pharmaceutically acceptable solvates thereof, and enantiomers thereof.

121. The method of claim 118, wherein at least one of R1 or R2 is a 5-methylene-8-hydroxyquinoline group.

122. The method of claim 118, wherein the compound is selected from the group consisting of 5,5′-(benzylazanediyl)bis(methylene)diquinolin-8-ol (2) (BPM18,725), 5,5′-(4-(methyl)benzylazanediyl)bis(methylene)diquinolin-8-ol (1) (BPM19,107), 5,5′-(4-(trifluoromethyl)benzylazanediyl)bis(methylene)diquinolin-8-ol (6) (BPM18,708), 5,5-(2-(trifluoromethyl)benzylazanediyl)bis(methylene)diquinolin-8-ol (3) (BPM19,178), 5,5′-(3-(trifluoromethyl)benzylazanediyl)bis(methylene)diquinolin-8-ol (7) (BPM19,189), 5,5′-(3,5-bis(trifluoromethyl)benzylazanediyl)bis(methylene)diquinolein-8-ol (8) (BPM18,201), 5,5′-(thiophen-2-ylmethylazanediyl)bis(methylene)diquinolin-8-ol (11) (BPM18,202), 5,5′-(3-iodobenzylazanediyl)bis(methylene)diquinolin-8-ol (10) (BPM19,200), 4-((bis((8-hydroxyquinolin-5-yl)methyl)amino)-methyl)cyclohexanecarboxylic acid (5) (BPM19,225) and 5,5′-cyclohexylmethylazanediyl-bis-[methylene)di(quinolin-8-ol)] (4) (BPM19,219).

123. The method of claim 118, wherein the compound is selected from the group consisting of 5,5′-(3,5-bis(trifluoromethyl)benzylazanediyl)bis(methylene)diquinolein-8-ol (8) (BPM18,201) and 5,5′-(thiophen-2-ylmethylazanediyl)bis(methylene)diquinolin-8-ol (11) (BPM18,202).

124. A compound of Formula III 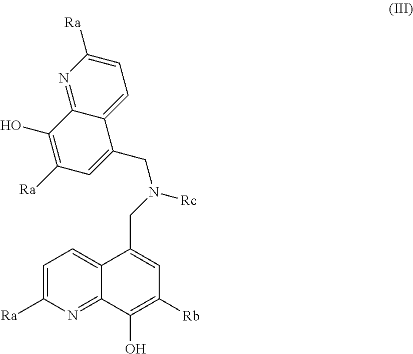
in which:
each Ra represents a C₁-C₆ alkyl group, unsubstituted or substituted with a halogen atom, a —NO₂, NH₂ or —OR group where R is a C₁ to C₄ alkyl group;
each Rb represents an allyl group, a C₂ to C₄ alkenyl or alkynyl group, propargyl or benzyl, the allyl, propargyl or benzyl being unsubstituted or substituted with a halogen atom, a —NO₂, NH₂ or —OR group where R is a C₁ to C₄ alkyl group;
Rc represents a hydrogen atom, a C₁ to C₁₀ alkyl group, a C₂ to C₄ alkenyl or alkynyl group or a 5-methylene-8-hydroxyquinoline group, a C₃ to C₆ cycloalkyl group, an aryl group, a —(CH₂)ₙ-heteroaryl comprising one or several heteroatoms selected from N, O and S, n being an integer between 0 and 4, a C₄ to C₆ —(CH₂)ₙ-heterocycloalkyl group in which the heteroatom represents N, O and S, n being an integer between 0 and 4, or alkylphenyl where the alkyl represents C₁ to C₁₀, the cycloalkyl, aryl, heteroaryl, heterocycloalkyl and phenyl groups being unsubstituted or substituted with one or two groups selected from F, Br, I and Cl, —CF₃, a C₁ to C₄ alkyl, COOH, CHO, COOR′ where R′ is a C₁ to C₄ alkyl group;
or Rc represents a group of formula (II) linked to the asymmetric carbon 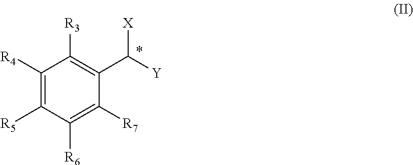
in which R₃, R₄, R₅, R₆ and R₇, independently of each other, represent a hydrogen atom, a C₁ to C₁₀ alkyl group, —CF₃, —NO₂, —NH₂, an N-5-methylene-8-hydroxyquinoline group, 1 or 2 halogen atoms chosen from F, Br, I and Cl or a group —O—R, R being a C₁ to C₄ alkyl group or —CF₃, X or Y represents a hydrogen atom, a C₁ to C₁₀ alkyl group, an aryl that is unsubstituted or substituted with a C₁ to C₁₀ alkyl group, —CF₃ or —NO₂,
or Rc represents a tert-butoxycarbonyl (Boc) group or —(CH₂)ₙ-phenyl, n being an integer between 1 and 5;
or Rc represents a Y—N—Y′ group where Y is selected from the group consisting of —(CH₂)ₙ—, n being an integer between 1 and 10, —(CH₂)ₙ-phenyl-(CH₂)ₚ—, the phenyl being unsubstituted or substituted with 1 or 2 halogen atoms selected from F, Br, I and Cl or with a C₁ to C₁₀ alkyl group, m and p respectively being a number between 1 and 4, and wherein Y′ is 5-methylene-8-hydroxyquinoline;
or Rc represents a —(CH₂)ₙ-naphtalene group, n being an integer between 1 and 10, the naphthalene group being unsubstituted or substituted with one or several groups selected from C₁ to C₁₀ alkyl groups, —CF₃ and O—R where R is a C₁ to C₁₀ alkyl group,
and pharmaceutically acceptable salts thereof, pharmaceutically acceptable solvates thereof, and enantiomers thereof.

125. A pharmaceutical composition comprising the compound of claim 124, in combination with a pharmaceutically acceptable carrier.

126. A method for identifying a compound which modulates the activity of a PPAR polypeptide, the method comprising: a) contacting said PPAR polypeptide with a test compound comprising an 8-hydroxyquinoline-methylene-N- group under conditions suitable for binding and/or modulation of the activity of said PPAR polypeptide; and b) detecting binding and/or modulation of the activity of said PPAR polypeptide by the compound.


SEQUENCE LISTING (4 sequences)

SEQ ID NO: 1 - protein, 477 aa, homo sapiens
MTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 2 - protein, 468 aa, homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 3 - protein, 477 aa, homo sapiens; identical to SEQ ID NO: 1

SEQ ID NO: 4 - protein, 462 aa, homo sapiens
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT